SEQUENCE LISTING (60 sequences)

SEQ ID NO: 1 - protein, 8 aa, synthetic construct
SSLVLADS

SEQ ID NO: 2 - protein, 8 aa, synthetic construct
SSNSVVGI

SEQ ID NO: 3 - protein, 8 aa, synthetic construct
SMSTYVPS

SEQ ID NO: 4 - protein, 8 aa, synthetic construct
STHTTSVL

SEQ ID NO: 5 - protein, 8 aa, synthetic construct
SSSSLLHY

SEQ ID NO: 6 - protein, 8 aa, synthetic construct
SHSYAGVL

SEQ ID NO: 7 - protein, 8 aa, synthetic construct
SSLTTLTA

SEQ ID NO: 8 - protein, 8 aa, synthetic construct
SLASAVSN

SEQ ID NO: 9 - protein, 8 aa, synthetic construct
SSALTVSH

SEQ ID NO: 10 - protein, 8 aa, synthetic construct
SVAVEAHH

SEQ ID NO: 11 - protein, 8 aa, synthetic construct
SHIGTVTG

SEQ ID NO: 12 - protein, 8 aa, synthetic construct
SVMTINNT

SEQ ID NO: 13 - protein, 8 aa, synthetic construct
SAQTLDRS

SEQ ID NO: 14 - protein, 8 aa, synthetic construct
SLIEWYSA

SEQ ID NO: 15 - protein, 8 aa, synthetic construct
SYMNNSVF

SEQ ID NO: 16 - protein, 8 aa, synthetic construct
SDTPTSPG

SEQ ID NO: 17 - protein, 8 aa, synthetic construct
SDPSQFPL

SEQ ID NO: 18 - protein, 8 aa, synthetic construct
SHLSYTMW

SEQ ID NO: 19 - protein, 8 aa, synthetic construct
SGNEDRYP

SEQ ID NO: 20 - protein, 8 aa, synthetic construct
SHDDIPHL

SEQ ID NO: 21 - protein, 8 aa, synthetic construct
SVDSNNEH

SEQ ID NO: 22 - protein, 8 aa, synthetic construct
SFGFMNLH

SEQ ID NO: 23 - protein, 8 aa, synthetic construct
STHVQNWL

SEQ ID NO: 24 - protein, 8 aa, synthetic construct
SNIMHSLN

SEQ ID NO: 25 - protein, 8 aa, synthetic construct
SNVIDPAN

SEQ ID NO: 26 - protein, 8 aa, synthetic construct
SGNSTSNA

SEQ ID NO: 27 - protein, 8 aa, synthetic construct
SSTVANDG

SEQ ID NO: 28 - protein, 8 aa, synthetic construct
SPNTNTLA

SEQ ID NO: 29 - protein, 8 aa, synthetic construct
SVFNVNNT

SEQ ID NO: 30 - protein, 8 aa, synthetic construct
SAYLDSLN

SEQ ID NO: 31 - protein, 8 aa, synthetic construct
SVSTTGVR

SEQ ID NO: 32 - protein, 8 aa, synthetic construct
SFSNIAIR

SEQ ID NO: 33 - protein, 8 aa, synthetic construct
SLSAVDVS

SEQ ID NO: 34 - protein, 8 aa, synthetic construct
SLLTTGPV

SEQ ID NO: 35 - protein, 8 aa, synthetic construct
SGSMIAMP

SEQ ID NO: 36 - protein, 8 aa, synthetic construct
SSAGMHVL

SEQ ID NO: 37 - protein, 8 aa, synthetic construct
SHNSQSIP

SEQ ID NO: 38 - protein, 8 aa, synthetic construct
SLGMTYGH

SEQ ID NO: 39 - protein, 8 aa, synthetic construct
SMPDASLS

SEQ ID NO: 40 - protein, 8 aa, synthetic construct
SQQTDSYP

SEQ ID NO: 41 - protein, 8 aa, synthetic construct
SASVHPLA

SEQ ID NO: 42 - protein, 8 aa, synthetic construct
SWTAYMSS

SEQ ID NO: 43 - protein, 8 aa, synthetic construct
SPQLTRNV

SEQ ID NO: 44 - protein, 8 aa, synthetic construct
SHAHSLSV

SEQ ID NO: 45 - protein, 8 aa, synthetic construct
SNPYSPSL

SEQ ID NO: 46 - protein, 8 aa, synthetic construct
SYLPDEFT

SEQ ID NO: 47 - protein, 8 aa, synthetic construct
STIQFTPQ

SEQ ID NO: 48 - protein, 8 aa, synthetic construct
STLSFTRM

SEQ ID NO: 49 - protein, 8 aa, synthetic construct
STWSYPRI

SEQ ID NO: 50 - protein, 8 aa, synthetic construct
SPLSFQAT

SEQ ID NO: 51 - protein, 8 aa, synthetic construct
SNFPSLSV

SEQ ID NO: 52 - protein, 8 aa, synthetic construct
SNKMDNTL

SEQ ID NO: 53 - protein, 8 aa, synthetic construct
SPIRGYTD

SEQ ID NO: 54 - protein, 8 aa, synthetic construct
SVDDYFTR

SEQ ID NO: 55 - protein, 8 aa, synthetic construct
SIDYMYAR

SEQ ID NO: 56 - protein, 8 aa, synthetic construct
STTVNTIA

SEQ ID NO: 57 - protein, 8 aa, synthetic construct
STYHTNSS

SEQ ID NO: 58 - protein, 8 aa, synthetic construct
SGSSFTMI

SEQ ID NO: 59 - DNA, 20 bp, synthetic construct
  source 1..20: mol_type = other DNA
ttttggagattttcaacgtg

SEQ ID NO: 60 - DNA, 20 bp, synthetic construct
  source 1..20: mol_type = other DNA
ccctcatagttagcgtaacg

(19) United States
(12) Patent Application Publication
(10) Pub. No.: US 2026/0357477 A1
(43) Pub. Date: Nov. 12, 2026

(54) BIOPANNING METHODS FOR IDENTIFYING PEPTIDES TARGETING GLIAL PHENOTYPES IN RAMIFIED, PRIMED, AND ACTIVATED BRAIN MICROGLIA AND ASTROCYTES

(71) Applicants: Northwestern University, Evanston, IL (US); University of Alberta, Edmonton (CA)

(72) Inventors: Kyle Koss, Evanston, IL (US); Kathryn Todd, Edmonton (CA); Ratmir Derda, Edmonton (CA); Jason Wertheim, Evanston, IL (US)

(21) Appl. No.: 19/110,341

(22) PCT Filed: Sep. 20, 2023
(86) PCT No.: PCT/US2023/074650

§ 371 Date: Mar. 10, 2025

Related U.S. Application Data
(60) Provisional application No. 63/408,187, filed on Sep. 20, 2022.

(51) Int. Cl. C40B 30/04; C12N 15/10; G01N 33/50

(52) U.S. Cl. CPC C40B 30/04; C12N 15/1037; G01N 33/5058

(57) ABSTRACT

Provided herein are biopanning methods for identifying peptides that bind to one or more glial cell subtypes, and peptides identified using the methods described herein. In some embodiments, provided herein are biopanning methods for identifying peptides that bind to glial cell subtypes including surveying glial cells, active glial cells, and/or primed glial cells.

### STATEMENT REGARDING RELATED APPLICATIONS

This application claims priority to U.S. Provisional Patent Application No. 63/408,187, filed Sep. 20, 2022, the entire contents of which are incorporated herein by reference.

### STATEMENT REGARDING FEDERAL FUNDING

This invention was made with Government support under grant number 5R01DK113168-02 awarded by the National Institutes of Health. The government has certain rights in the invention.

### SEQUENCE LISTING

The text of the computer readable sequence listing filed herewith, titled “NWEST-41146-601_SQL”, created Sep. 20, 2023, having a file size of 52,906 bytes, is hereby incorporated by reference in its entirety.

### FIELD

Provided herein are biopanning methods for identifying peptides that bind to one or more glial cell subtypes, and peptides identified using the methods described herein. In some embodiments, provided herein are biopanning methods for identifying peptides that bind to glial cell subtypes including surveying glial cells, active glial cells, and/or primed glial cells.

### BACKGROUND

Neuroinflammatory disorders of the central nervous system (CNS) contribute extensively to global mortality and morbidity in the forms of physical and mental impairment. A broad spectrum of such illnesses includes traumatic injury to the brain or spinal cord, stroke, several neuropsychiatric disorders, and effectively every neurodegenerative and prion disease. Current understanding of the cause, pathology, and symptoms of these diseases is extremely varied; however, they all share one common inflammatory component. Microglia, resident CNS immune cells that normally regulate healthy function, create, and perpetuate inflammation resulting in permeability of the blood-brain barrier, immune cell recruitment, and toxic release of oxidative species and cytokines that exacerbate cell stress leading to death of neighboring neural cells. The result of these cascades ranges from withdrawal of neural processes, degradation of neural circuitry, and broad life-changing deficits in cognition, memory, and motor control. Notably, there are no cures for neuroinflammatory diseases, and many are progressive over time, exceptionally prolonging patient suffering.

Inflammation is a complex tissue response to harmful stimuli, such as pathogens and damage, with the goals of protection, clearance of damaged cells, and healing. Microglia are thought to acquire multiple and diverse functional roles which can be represented as several phenotypes across a spectrum of behavior. These states include a surveying phenotype, a reactive phenotype, and a primed phenotype. Ideally, treatments for neuroinflammatory conditions should include both a suppression strategy for the inflammatory component, and the promotion of rescue and regeneration of damaged and lost neural cells. As microglia are uniquely capable in regulating both processes. especially in conjunction with astrocytes, drugs and ligands that target these phenotypically diverse cells are of unique interest. Demonstrated anti-inflammatory therapeutics, which include tetracycline antibiotics and corticosteroids, such as minocycline and dexamethasone respectively, are capable of attenuating inflammation and have some neuroprotective effects, However, these drugs are almost always linked to unwanted long term side effects including depression, diabetes mellitus, and even compromised immunity, Accordingly, what is needed are methods for identifying potential candidate treatments, in particular that can differentially bind to desired microglia phenotypes, for improved treatments of neuroinflammatory disorders.

### SUMMARY

In some aspects, provided herein are biopanning methods. The biopanning methods described herein find use in methods for identifying peptides that specifically bind to one or more glial cell subtypes. In some embodiments, the method comprises providing a population of surveying glial cells, a population of reactive glial cells, and a population of primed glial cells, and using a phage display technique to identify peptides that bind to one or more of the populations of cells. In some embodiments, the method comprises contacting each population of glial cells with a phage library, isolating phage-bound glial cells, amplifying phage-bound glial cells, and sequencing the phage bound-glial cells.

In some embodiments, the population of reactive glial cells is generated by culturing glial cells with a composition comprising lipopolysaccharide (LPS). In some embodiments, the population of primed microglial cells is generated by culturing glial cells in a composition comprising lipopolysaccharide (LPS), followed by culturing the glial cells in a composition comprising dexamethasone. For example, in some embodiments the population of primed microglial cells is generated by culturing the glial cells in the composition comprising LPS for 4-8 hours, followed by culturing the glial cells in the composition comprising dexamethasone for 12-36 hours.

Any suitable phage library may be used. In some embodiments, the phage library comprises a filamentous phage library. In some embodiments, the phage library comprises an M13 phage library.

In some embodiments, the phage library comprises a plurality of phages, each phage displaying one or more peptides comprising 7 randomized amino acids. In some embodiments, the one or more peptides each comprise a serine residue (S) and 7 randomized amino acids. In some embodiments, the one or more peptides have at least 62.5% sequence identity to a sequence provided herein. In some embodiments, the one or more peptides have at least 62.5% sequence identity to a sequence provided in Table 3. In some embodiments, the one or more peptides have at least 62.5% sequence identity (e.g. at least 62.5%, at least 75%, at least 87.5%, or 100% sequence identity) to a sequence selected from SEQ ID NO: 1-SEQ ID NO: 58. In some embodiments, the one or more peptides each have at least 87.5% sequence identity to a sequence selected from SEQ ID NO: 1-SEQ ID NO: 58.

### BRIEF DESCRIPTION OF THE DRAWINGS

FIG. 1 shows the overall schematic of the glial treatments, cell-phage sorting, and sequencing. The phenotypes of microglia and astrocytes in states of neuroinflammation are shown with respective treatments. An untreated condition suggests a ramified or surveying phenotype outcome. A 6-hour inflammatory stimulus (LPS) promotes an activated phenotype with reactive microglia and astrogliosis. A recovery for 18 hours with dexamethasone (Dex) promotes a primed phenotype. A SX7 filamentous phage library was further treated with these cell populations, collected, and sorted via Fluorescent-Assisted Cell Sorting (FACS), based on fluorescently tagged SX7 bound microglia and astrocytes populations. These phage-bound cells were amplified in bacterial culture, and peptide-related DNA sequences were characterized with Next-generation Sequencing (NGS).

FIGS. 2A-2H show confocal images and immunofluorescent quantification and of primary rat iNOS labelled microglia and mixed glia. Samples were treated with 100 ng/mL LPS for 6 hrs, followed by 18 hrs 10 μM Mino/10 μM Dex/CM, and a 24 hrs 100 ng/mL treatment with IFNγ. Cells were quantified by percent positive counts. FIG. 2A shows confocal images of control microglia. FIG. 2B shows confocal images of microglia treated with LPS. FIG. 2C shows confocal images of microglia treated with LPS and recovered with CM. FIG. 2D shows confocal images of microglia treated with LPS and recovered with Dex. FIG. 2E shows confocal images of microglia treated with LPS and recovered with Mino. Expression of iNOS is quantified in FIG. 2F. For FIGS. 2A-2E, blue stain indicates nuclei, red stain indicates Iba1, and green stain indicates iNOS. FIG. 2G shows confocal images of control mixed glia. FIG. 2H shows confocal images of mixed glia treated with LPS. FIG. 2I shows confocal images of mixed glia treated with LPS and recovered with CM. FIG. 2J shows confocal images of mixed glia treated with LPS and recovered with Dex. FIG. 2K shows confocal images of mixed glia treated with LPS and recovered with Mino. Expression of iNOSin these missed glial populations is quantified in FIG. 2L. For FIGS. 2G-2K, blue stain indicates nuclei, red stain indicates Iba1, green stain indicates GFAP, and white indicates iNOS. For all images the scale bar is 50 m. n=5 for all groups derived from a weekly prep of glial cells and data analyzed represent mean±SEM. A p value of ≤0.05 is significant, where + and * represents group-wise and pair-wise comparisons across Veh, CM, Dex, and Mino groups, respectively.

FIGS. 3A-3H show inflammatory molecular assays for primary rat isolated microglia and mixed glia, including nitrite, and ELISA results for TNF-α, IL-1β, and IL-10 release normalized to cell count (stained cell populations). Samples were treated with 100 ng/mL LPS for 6 hrs, followed by 18 hrs 10 μM Mino/10 μM Dex/CM, and a 24 hrs 100 ng/mL treatment with IFNγ. FIG. 3A show nitrite release in isolated microglia. FIG. 3B shows nitrite release in mixed glia. FIG. 3C shows IL-1β1 release in isolated microglia. FIG. 3D shows IL-1β release in mixed glia. FIG. 3E shows TNF-α release in isolated microglia. FIG. 3F shows TNF-α release in mixed glia. FIG. 3G shows IL-10 release in isolated microglia. FIG. 3H shows IL-10 release in mixed glia. n=5 for all groups and data analyzed represent mean±SEM. A p value of ≤0.05 is significant, where + and * represents group-wise and pair-wise comparisons across Veh, CM, Dex, and Mino groups, respectively.

FIGS. 4A-4H show immunofluorescent quantification of microglial phagocytosis labelled with CNPase (myelin) uptake in mixed glia. Samples were treated with 100 ng/mL LPS for 6 hrs, followed by 18 hrs 10 μM Mino/10 μM Dex/CM, and a 24 hrs 100 ng/mL treatment with IFNγ. n=5 for all groups derived from a weekly prep of glial cells and data analyzed represent mean±SEM. A p value of ≤0.05 is significant, where + and * represents group-wise and pair-wise comparisons across Veh, CM, Dex, and Mino groups, respectively. Oligodendrocytes are quantified in each condition in FIG. 4A. FIG. 4B shows a representative confocal image of control microglia. FIG. 4C shows a representative confocal image of microglia treated with LPS. FIG. 4D shows a representative confocal image of microglia treated with INFγ. FIG. 4E shows a representative confocal image of microglia treated with LPS/INFγ. FIG. 4F shows a representative confocal image of microglia treated with LPS and recovered with CM. FIG. 4G shows a representative confocal image of microglia treated with LPS and recovered with Dex. FIG. 4H shows a representative confocal image of microglia treated with LPS and recovered with Mino. Image labels are Iba1 in red for microglia, CNPase for oligodendrocytes/myelin in blue, GFAP in green for astrocytes, and Hoescht white for nuclei. Arrows indicate example CNPase particulates overlapping Iba1, where additional panels provide contrast. The scale bar is 50 μm.

FIGS. 5A-5C show representative confocal images of glia treated with 5×10¹² pfu SX7 library. FIG. 5A shows untreated surveying microglia, FIG. 5B shows activated microglia (treated with 100 ng/mL LPS for 6 hours), and FIG. 5C shows primed microglia (treated with 100 ng/mL LPS for 6 hrs, followed by 18 hrs 10 μM Dex). Labels are blue (nuclei), green (Iba1), red (GFAP), and white (SX7). The scale bar is 50 m. The inserts in each image show post-FACS sorted cell images of SX7 tagged for microglia (Bi, Bii, and Biii) and astrocytes (Ci, Cii, and Ciii). The inserts show phase contrast with green GLAST/CD11b and red SX7.

FIGS. 6A-6C show bioinformatic presentation of sequences obtained from next-generation sequencing (NGS), which hit sequence clustering was based on features described by the BLOSUM62 matrix. The clustered peptide sequences are characterized by normalized counts per million and were filtered based on the presence in the experimental group compared to its counterpart control groups. Significant enrichment is p<0.05, where n>4. FIG. 6A shows exemplary volcano plots microglia Ctr (surveying untreated control) versus LPS, Dex, and astrocyte Ctr. LPS, and Dex. The highlighted section (top right-hand side) is of most interest. This region contains peptide sequences that were enriched (i.e., was observed with a larger Read number) and had significantly lower p-value (i.e., higher statistical significance). FIG. 6B shows heat maps that summarize the clustering and conformity to filtering condition for microglia. Top panel shows control microglia, middle panel shows LPS-treated microglia, and bottom panel show Dex treated microglia. FIG. 6C shows heat maps that summarize the clustering and conformity to filtering condition for astrocytes. Top panel shows control astrocytes, middle panel shows LPS-treated astrocytes, and bottom panel show Dex treated astrocytes.

FIG. 7 shows In-Cell Western assays to detect biotin C-terminus conjugated peptides in populations of primary rat (A) microglia and (B) astrocyte in mixed populations. Samples were treated with 100 ng/mL LPS for 6 hrs, followed by 18 hrs 10 μM Dex. Only significant binders are shown. Binding treatments are compared to corresponding control treatments in the following configuration of BindTreat/ContA/ContB: Ctr/LPS/Dex, LPS/Dex/Ctr, and Dex/Ctr/LPS.BindTreat, ContA, and ContB refer to the experimental group and swaps of the other treatments making them separate controls, respectively. Sample size is n=6 for all groups and analyzed data represent a mean±SEM. Significance compared clusters of experimental binders (group-wise) to both respective control treatments. A p value of ≤0.05, ≤0.01, and ≤0.001 were used to present significance or robust specificity of the binder and were presented as +, ++, and +++, respectively, noted in Tables 2 and 3.

FIG. 8A shows an example of FACSAria III gating workflow for collecting SX7 adhered to microglia and astrocytes. Gates include whole cell (box A), S-Singlet (box B), F-Singlet (box C), exclusion of auto-fluorescent cells within the indicated bounding box(NOT-Fluor) (box D), live cells (excluding cells positive for Sytox blue: NOT-SytBl) (box E), PECy-7 CD11b positive cells (box F), PE GLAST positive cells (box G), and AF647 SX7 bound CD11b positive cells (AF647-SX7-M)) (box H), and AF647 SX7 bound GLAST positive cells (AF647-SX7-A) (box I). A summary of the mean percentage of each gated population (n=4).

FIGS. 9A-9I show example gates for different cell populations. FIG. 9A shows exemplary gates for CD11b microglia for surveying (left), activated (middle), and primed conditions (right). Results are quantified in FIG. 9B. FIG. 9C shows quantification of gating for live cells. FIG. 9D shows exemplary gates for GLAST astrocytes for surveying (left), activated (middle), and primed conditions (right). Results are quantified in FIG. 9E. FIG. 9F shows exemplary gates for GLAST astrocytes sub-gated for AF-647-SX7 for surveying (left), activated (middle), and primed conditions (right). Results are quantified in FIG. 9G. FIG. 9H shows exemplary gates Cd11b microglia sub-gated for AF-647-SX7 for surveying (left), activated (middle), and primed conditions (right). Results are quantified in FIG. 9I. Sample size is n=4 for all groups and data analyzed represent mean±SEM. A p value of ≤0.05 is significant, where + represent pair-wise comparisons within groups, respectively.

FIG. 10A is a panel showing populations of flow-cytometrically sorted events and FIG. 10B is a graph showing SX7 titre of the sorted events. FIG. 10D is a blot showing polymerase chain reaction (PCR) amplified DNA concentration characterized by gel electrophoresis. Results are quantified in FIG. 10C. Events are characterized based on the total cells collected from the gated populations. Titres are calculated via a serial dilution of sample, where green plaques can be counted (X-Gal/IPTG). DNA bands were quantified based on a DNA standard. Sample size is n=4 for all groups and analyzed data represent a mean±SEM. A p value of ≤0.05 is significant, where + represents pair-wise comparisons within groups, respectively.

FIG. 11 is a volcano plot summary of sequences obtained for next-generation sequencing (NGS), which hit sequence clustering was based on features described by the BLOSUM62 matrix. Significantly enrichment is p<0.05, where n>4. Within each plot, the highlighted section (top right-hand side) is of most interest. This region contains peptide sequences that were enriched (i.e., was observed with a larger Read number) and had significantly lower p-value (i.e., higher statistical significance).

FIG. 12 shows in-cell Western assays to detect biotin C-terminus conjugated peptides in populations of primary rat astrocytes in mixed populations. Samples were (A) untreated, (B) treated with 100 ng/mL LPS for 6 hrs, followed by (C) 18 hrs 10 μM Dex. Only significant binders are shown. Binding treatments are compared it corresponding control treatments in the following configuration of BindTreat/ContA/ContB: Ctr/LPS/Dex, LPS/Dex/Ctr, and Dex/Ctr/LPS. n=6 for all groups and data analyzed represent mean±SEM. Significance compared clusters of experimental binders (group-wise) to both respective control treatments. A p value of ≤0.05, ≤0.01, and ≤0.001 were used to present significance or robust specificity of the binder and were presented as +, ++, and +++, respectively.

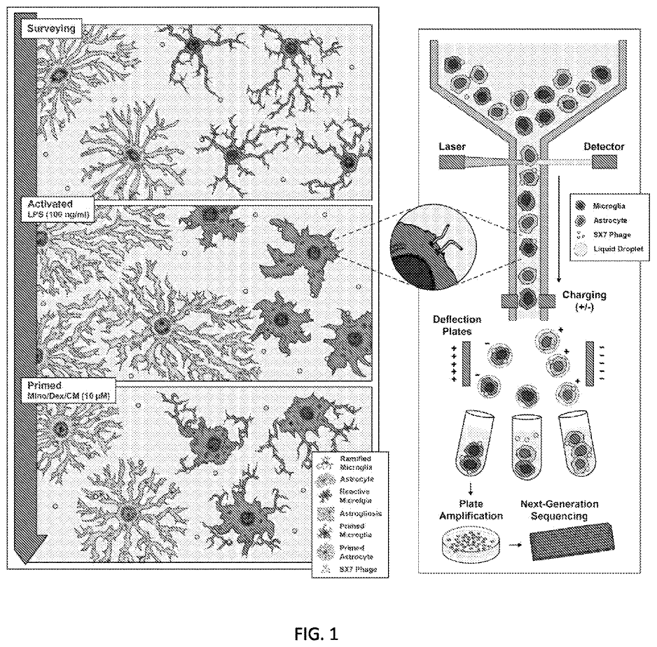

### DETAILED DESCRIPTION

Provided herein biopanning methods and peptides identified using the same.

In some aspects, provided herein is a biopanning method for identifying peptides that bind to one or more glial cell subtypes. The methods may be performed on any suitable glial cell, including microglia and/or astrocytes.

In some embodiments, the method comprises providing populations of glial cell subtypes, and performing phage-display to identify peptides that bind to one or more of the populations of glial cell subtypes. In some embodiments, the method comprises providing a population of surveying glial cells, a population of reactive glial cells, and a population of primed glial cells, and identifying peptides that bind to one or more populations using phage-display. In some embodiments, the populations of reactive glial cells, primed glial cells, and surveying glial cells are separate cell populations (e.g. isolated cell populations, not mixed). Accordingly, in some embodiments the population of surveying glial cells is an isolated population of glial cells, the population of reactive glial cells is an isolated population of glial cells, and the population of primed glial cells is an isolated population of glial cells.

In some embodiments, the populations of reactive, primed, and surveying glial cells are identified based upon morphology, expression of markers, and/or other characteristics. In some embodiments reactive glial cells are flattened. In some embodiments, reactive glial cells are round, amoeboid cells. In some embodiments, reactive glial cells have relatively high release of one or more pro-inflammatory proteins compared to that of primed and surveying glial cells. Exemplary pro-inflammatory proteins include cytokines such as IL-1β, TNF-α, STAT3, IL-6, IL-12, IL-23, and/or IL-10. For example, in some embodiments reactive glial cells release relatively high levels TNF, IL-1β, and/or IL-10. As another example, in some embodiments reactive glial cells release relatively high levels of nitric oxide (NO) compared to primed and/or surveying glial cells. Release of pro-inflammatory moieties (e.g. cytokines, nitric oxide, etc.) can be evaluated by any suitable method as known in the art. For example, release of pro-inflammatory moieties from a population of cells can be evaluated by western blow, ELISA, or other suitable assay in order to determine whether a population of glial cells comprises a reactive, primed, or surveying phenotype.

In some embodiments reactive glial cells have relatively high expression of the pro-inflammatory intracellular protein inducible nitric oxide synthase (iNOS) compared to expression in primed and/or surveying glial cells. In some embodiments, expression of iNOS in reactive glial cells is substantially heterogenous (e.g. expressed throughout the cell). In some embodiments, primed glial cells have an intermediate morphology, such as a less flattened nature than reactive glial cells but a smaller number/amount of observable processes (e.g. extension from the cell body) compared to those in surveying glial cells. In some embodiments, primed glial cells have an intermediate expression of iNOS, namely lower levels of iNOS expression than in reactive glial cells but higher levels than in surveying glial cells. In some embodiments, expression of iNOS in primed glial cells is less dispersed throughout the cell (e.g. less heterogenous) than the expression of iNOS in reactive glial cells. In some embodiments, surveying microglia have bifurcated processes and elongated extensions. In some embodiments, surveying microglia have little to no detectable iNOS expression. The morphology of cells and/or expression of cellular markers such as iNOS can be evaluated by any suitable technique, including imaging techniques (e.g., confocal microscopy), to determine whether a population of glial cells comprises a reactive, primed, or surveying phenotype.

In some embodiments, the population of reactive glial cells is generated by culturing glial cells with a composition comprising lipopolysaccharide (LPS). In some embodiments, the population of reactive glial cells is generated by culturing the glial cells with the composition comprising LPS for about 4-8 hours. In some embodiments, the population of reactive glial cells is generated by culturing the glial cells with the composition comprising LPS for about 4 hours, about 5 hours, about 6 hours, about 7 hours, or about 8 hours. In some embodiments, the population of reactive glial cells is generated by culturing the glial cells with the composition comprising LPS for about 6 hours. In some embodiments, the concentration of LPS is about 10 to about 200 ng/mL. In some embodiments, the concentration of LPS is about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, or about 200 ng/mL. In some embodiments, the concentration of LPS is about 100 ng/mL.

In some embodiments, the population of primed glial cells is generated by culturing glial cells in a composition comprising lipopolysaccharide (LPS), followed by culturing the glial cells in a composition comprising dexamethasone (Dex). In some embodiments, the population of primed microglial cells is generated by culturing the glial cells in the composition comprising LPS for about 4-8 hours (e.g., about 4 hours, about 5 hours, about 6 hours, about 7 hours, or about 8 hours), followed by culturing the glial cells in the composition comprising dexamethasone for 12-36 hours. In some embodiments, the population of primed glial cells is generated by culturing glial cells in a composition comprising LPS for about 4-8 hours, followed by culturing the glial cells in a composition comprising dexamethasone for about 20-28 hours. In some embodiments, the population of primed microglial cells is generated by culturing the glial cells in the composition comprising LPS for about 6 hours, followed by culturing the glial cells in the composition comprising dexamethasone (Dex) for about 24 hours. Suitable concentrations of LPS are described above. In some embodiments, the concentration of Dex is about 1 μM to about 20 μM. In some embodiments, the concentration of Dex is about 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, or about 20 μM. In some embodiments, the concentration of Dex is about 10 μM.

In some embodiments, the population of surveying glial cells is untreated (e.g. not treated with LPS and/or dexamethasone).

In some embodiments, the method comprises providing a population of surveying glial cells, a population of reactive glial cells, and a population of primed glial cells, and identifying peptides that bind to one or more glial cell populations using phage-display. Phage display refers to a technique used to study interactions between two moieties (e.g. protein-protein interactions, protein-peptide interactions, protein-DNA interactions). Generally, phage display uses bacteriophages to display a protein or proteins of interest on the surface of a bacteriophage, also referred to as a phage (e.g. virus that infects bacteria). In phage display, a gene encoding a protein of interest is inserted into a phage coat protein gene, causing the bacteriophage to display the protein on the outside while the gene encoding the protein is held within the bacteriophage. Phages displaying the protein of interest can then be used to screen for proteins, peptides, DNA sequences, cell types, etc. that interact with the displayed protein(s).

In some embodiments, phage display uses a phage library (also referred to as a phage display library) wherein each phage in the library displays one or more proteins. In some embodiments, phage display uses a phage library wherein each phage in the library is engineered to express a randomized peptide sequence on its surface. Phage libraries can thus be engineered to express a wide diversity of randomized peptides and enable high-throughput screening of many possible interactions (e.g. protein-protein interactions) simultaneously. In the methods provided herein, a phage library is used to identify sequences that bind to one or more microglial cell types. In some embodiments, a phage library (e.g. a library wherein each phage expresses a randomized peptide sequence) is used to determine which peptide, if any, expressed on the surface of a phage interacts with reactive, surveying, and/or microglial cell types. In some embodiments, the methods presented herein use phage display to determine which peptide sequences, if any, on the surface of a phage within the phage library bind to surveying, reactive, or microglial cells, with many possible binders being evaluated simultaneously.

Any suitable phage library may be used. Suitable phage libraries include, for example, filamentous phage libraries. A “filamentous phage” or a “filamentous bacteriophage” refers to a phage having a distinctive filamentous shape, about 6 mm in diameter and about 1,000-2,000 nm long. In some embodiments, the filamentous phage is an M13 bacteriophage. In some embodiments, the phage library is a T7 phage library. The methods provided herein utilized a custom 7 amino acid presenting phage library on M13 bacteriophage. This custom library is referred to herein as the SX7 phage library The SX7 phage library displays a consistent serine reside (S) (e.g. serine is present in all of the randomized peptide sequences presented on the phage) and 7 additional randomized amino acids. The custom amino acid presenting phage, SX7, is shown herein to bind to reactive microglia, primed microglia, and surveying microglia (see FIG. 5). In some embodiments, the phage library comprises phages presenting peptides (e.g. displaying peptides on the surface) comprising 7 randomized amino acids. In some embodiments, the phage library comprises phages presenting peptides comprising a consistent amino acid residue, followed by 7 randomized amino acids. It is understood that the number of amino acids in the randomized peptide sequences presented on the phage can vary. The number of amino acids presented in each randomized peptide on the phage surface should be balanced to achieve specific binding to target sequences while also facilitating creation of the phage library. In some embodiments, the phage library comprises phages presenting randomized peptides of 4-20 amino acids in length. In some embodiments, the phage library comprises phages presenting randomized peptides of 4-20, 5-18, 6-16, 7-14, or 8-12 amino acids in length.

In some embodiments, the phage library comprises a plurality of phages, each phage displaying one or more peptides comprising 7 randomized amino acids. In some embodiments, the one or more peptides each comprise a serine residue (S) and 7 randomized amino acids. In some embodiments, the phage library comprises a plurality of phages, and each phage displays one or more peptides having at least 62.5% sequence identity to a sequence provided herein. In some embodiments, the phage library comprises a plurality of phages, each phage displaying one or more peptides having at least 62.5% sequence identity to a sequence provided in Table 3. In some embodiments, the phage library comprises a plurality of phages, each phage displaying one or more peptides having at least 62.5% sequence identity (e.g. at least 62.5%, at least 75%, at least 87.5%, or 100% sequence identity) to a sequence selected from SEQ ID NO: 1-SEQ ID NO: 58. In some embodiments, the phage library comprises a plurality of phages, each phage displaying one or more peptides having at least 75% sequence identity to a sequence selected from SEQ ID NO: 1-SEQ ID NO: 58. In some embodiments, the phage library comprises a plurality of phages, each phage displaying one or more peptides having at least 87.5% sequence identity to a sequence selected from SEQ ID NO: 1-SEQ ID NO: 58. In some embodiments, the one or more peptides displayed on the phages in the phage library are exactly 7 amino acids in length. In some embodiments, the phage library comprises phages displaying additional peptides that do not have at least 62.5% sequence identity to one or more of SEQ ID NO: 1-SEQ ID NO: 58.

In some embodiments, randomized peptides presented on the surface of a phage share a common tag, such that phage-displayed peptides can be subsequently identified or isolated. For example, randomized peptides in a phage library may share a common nucleic acid tag (e.g. a common short sequence of nucleic acids) which can subsequently be identified after binding to a desired cell type, such as by sequencing using primers specific for the tag sequence.

In some embodiments, the method comprises providing a population of surveying glial cells, a population of reactive glial cells, and a population of primed glial cells, contacting each population with a phage library, and isolating phage-bound glial cells. Phage-bound glial cells (e.g., phage-bound surveying glial cells, phage-bound reactive glial cells, and/or phage-bound primed glial cells) can be isolated using any suitable technique. In some embodiments, phage-bound glial cells are isolated using cell sorting-based techniques, including flow cytometry, fluorescence-activated cell sorting (FACs), magnetic-activated cell sorting, buoyancy-activated cell sorting, microfluidics, centrifugation, filtration, and the like. In some embodiments, the method further comprises amplifying the phage-bound glial cells. For example, the cells may be amplified in a bacterial culture (e.g., E. coli culture). In some embodiments, “amplifying the phage-bound glial cells” refers to collecting the cells or a portion thereof (e.g., cell pellet, supernatant) and amplifying in the bacterial culture.

In some embodiments, the method further comprises sequencing the phage-bound glial cells. Sequencing may be performed after amplifying the phage-bound glial cells. In some embodiments, sequencing the phage-bound glial cells comprises isolating nucleic acid from the cells, and sequencing the nucleic acid. For example, in some embodiments sequencing the phage-bound glial cells comprises isolating DNA from the phage-bound glial cells and sequencing the DNA. In some embodiments, sequencing comprises next-generation sequencing (NGS). NGS refers to massively-parallel sequencing technique. In some embodiments, NGS involves fragmenting nucleic acids into multiple pieces, adding adapters, sequencing libraries, and re-assembling libraries to form a genomic sequence. Sequencing can be used to identify peptides that bind to one or more of the populations of glial cell sub-types.

The methods described herein can be used to identify peptides that differentially bind to glial cell sub-types, which may be beneficial for promoting certain beneficial aspects of the inflammatory response or suppressing other detrimental aspects of the inflammatory response. The biopanning methods described herein therefore may be used to identify peptides that can be used in a therapeutic setting, including for the treatment or management of inflammatory disorders of the central nervous system. In some aspects, provided herein is a peptide identified using a biopanning method as described herein. Suitable peptides identified herein include those listed in the tables corresponding to Example 1, below.

### EXAMPLES

### Example 1

### Materials and Methods

Dulbecco's Modified Eagle Medium: Nutrient Mixture F12 (DMEM F12), Hank's Balanced Salt Solution (HBSS), fetal bovine serum (FBS), penicillin streptomycin (PS), 0.25% trypsin-ethylenediaminetetraacetic acid (Trypsin-EDTA), and normal horse serum (NHS) were purchased from Gibco (Life Technologies, Burlington, ON, Canada). Poly-L-lysine hydrobromide (PLL), glucose, dexamethasone, minocycline, polyethylene glycol 300 (PEG300), PEG8000, and lipopolysaccharide (LPS) were purchased from Sigma Aldrich (St. Louis, MO, USA). Interferon gamma was purchased from Peprotech (Montreal, QC, Canada). Polystyrene 75 cm³ flasks were purchased from Corning (Corning, NY, USA). Polystyrene 12-well cell culture plates were purchased from Greiner Bio-One (Frickenhausen, Germany). 5 ml FACS tubes, TrypLE Express, Sytox Blue, Petri Dishes 100 mm×15 mm, X-GAL, IPTG, sodium chloride, magnesium chloride, hexahydrate, UltraPure agarose, 50 bp ladder, Phusion® HF reaction buffer, DNA polymerase, dNTP Mix, Sybr Safe DNA gel stain (SYBR1), dimethyl sulfoxide (DMSO), glycerol, glacial acetic acid, tris base, EDTA, and nuclease-free water were purchased from ThermoFisher (Mississauga, ON, Canada). Bacto Tryptone, Yeast Extract, Bacto Agar were purchased from BD Biosciences (Mississauga, ON, Canada). NF10 forward prime (e.g., TTTTGGAGATTTTCAACGTG (SEQ ID NO: 59)) and ‘96’ reverse primer (e.g., CCCTCATAGTTAGCGTAACG (SEQ ID NO: 60)) were produced by Integrated DNA Technologies (Coralville, IA, USA). Low molecular weight DNA ladders and 6× gel loading dyes were purchased from New England Biolabs (Whitby, ON, Canada).

The following primary antibodies were used: mouse anti-iNOS (Santa Cruz Biotechnology, Santa Cruz, CA, USA), PE-Cy7 labelled rat anti-CD11b (ThermoFisher), PE labelled mouse anti-GLAST (MiltenyiBiotec, BergischGladbach, Germany), rabbit anti-Iba1 (Wako, Osaka, Japan), and chicken anti-GFAP (Abeam).

The following secondary antibodies were used: donkey anti-mouse Alexa Fluor (AF) 488 (Invitrogen, Burlington, ON, Canada), goat anti-chicken AF555 (Invitrogen), donkey anti-mouse AF647 (Invitrogen), donkey anti-rabbit AF647 (Invitrogen), IR Dye 800RD Streptavidin (Li-Cor, Lincoln, NE, USA), and donkey anti-rabbit IR Dye 680RD (Li-Cor, Lincoln, NE, USA). Hoescht 33342 was purchased from Molecular Probes (Life Technologies, Burlington, ON, Canada). Fluormount-G was purchased from Southern Biotech (Birmingham, AL, USA). The antibodies are summarized in Table 1.

A peptide library of the sequences was purchase from Genscript with 5 mg batches at >90% purity. Each peptide had a C-terminal glycine spacer and a biotin-conjugated lysine (-G-G-K^{biotin}), for subsequent use with in-cell western labelling.

TABLE 1
Antibodies used in Throughout Study

| | | | Catalogue |
|---|---|---|---|
| Antibodies | Application | Supplier | Number |
| Mouse anti-iNOS | IF | Santa Cruz | sc-7271 |
| | | Biotechnology | |
| Rabbit anti-Iba1 | IF/ICW | Wako | 019-19741 |
| Chicken anti-GFAP | IF/ICW | Abcam | ab4674 |
| PE-Cy7 labelled rat | FACS | ThermoFisher | 25-0112-82 |
| anti-CD11b | | | |
| PE labelled mouse | FACS | MiltenyiBiotec | 130-095-822 |
| anti-GLAST | | | |
| Donkey anti-mouse | IF | Invitrogen | A-21202 |
| AF488 | | | |
| Goat anti-chicken | IF | Invitrogen | A-21437 |
| AF555 | | | |
| Donkey anti-mouse | IF | Invitrogen | A-31571 |
| AF647 | | | |
| Donkey anti-rabbit | IF | Invitrogen | A-31573 |
| AF647 | | | |
| IR Dye 800RD | ICW | Li-Cor | 926-32230 |
| Streptavidin | | | |
| IR Dye 680RD donkey | ICW | Li-Cor | 926-68073 |
| anti-rabbit | | | |

Immunofluorescence (IF), In-Cell Western (ICW), Flow Assisted Cell Sorting (FACS)

### Phage Library Amplification, Purification, and Fluorescent Labelling

The filamentous M13 phage SX7 was an in-house made phage chosen to allow potential future ligation at the N-terminal serine residue via oxime bond formation. Large scale amplification of phage was performed with E2738 Escherichia coli bacteria. Briefly, overnight culture (250 μL) was transferred into 20 mL of liquid broth (LB) in a 250 mL Erlenmeyer flask, culture in a 37° C. shaker to reach OD₆₀₀=0.2. Phage at ~5 μL of 2×10¹³ pfu/mL was added and then the media was incubated at 37° C. with 200 rpm shaking for 30 min. The incubated phage and bacteria were transferred into 1 L pre-warmed LB in a 5 L Erlenmeyer flask and incubated at 37° C. with 200 rpm shaking for 4.5 hour. The culture was centrifuged for 10 min at 6,000×g at 4° C., the supernatant was topped up with 1/6 volume of 6×PEG8000/NaCl (~2 mL) to make a final concentration of 0.5M NaCl and 5% PEG8000, and the solution was precipitated 2 hr-overnight at 4° C. The PEG precipitate was collected by centrifugation 14,000×g for 30 min at 4° C., re-suspended in 1 mL PBS (pH 7.4), and micro-centrifuged, max speed, for 5 min at 4° C. The supernatant was re-suspended with 1/6 volume 6×PEG8000/NaCl (~200 L) for 1 hr on ice and the precipitate was microcentrifuged at max speed for 30 min at 4° C. The precipitated pellet is the purified phage, re-suspended the pellet in 500 μL of PBS/5% (w/v) glucose.

Additional plate culture of phage was performed for titre applications. In a 5 ml test tube, 200 μl LB to the phage sample, 200 μl of overnight E. coli ER2738 culture, and 3 ml of warm liquid agar (low concentration agar LA media), were mixed and poured into a 10 cm petri dish with solid IPTG/X-gal agar. Cultures were incubated overnight at 37° cand titre counted based on the number of green coloured colony plaques. A serial dilution of phage sample was used to calibrate the titre.

SX7 phage was labelled with an AF647 NHS Ester kit (ThermoFisher, Mississauga, ON, Canada). Phage at a titre greater than 10¹¹ pfu in 50 M NaCO₃/NaHCO₃ (pH 9.0) was incubated with 20 μg/ml fluorescent dye for 2 hours without light at room temperature. Samples were immediately dialysed at 4° C. with 10K MW tubing in PBS overnight, with solution changes at 1 hour, 3 hours, and the following morning before collection. Phages were used within 2 weeks, stored a 4° C.

### Primary Microglial Cell Culture and Treatments

Animal protocols were approved by the Animal Care and Use Committee at the University of Alberta and Institutional Animal Care and Use Committee at Northwestern University and conducted in accordance with animal ethics guidelines. Mixed glial cells (microglia, astrocytes, oligodendrocytes) were harvested from postnatal Day 1 male Sprague Dawley rats. The rats were decapitated, and their brains removed and dissected in Hank's balanced salt solution with 1% PS, to remove meninges and vasculature. Tissues were then dissociated in 0.25% Trypsin-EDTA (2 mL/brain) at 37° C. for 25 minutes, washed with complete culture media (DMEM F12/10% FBS/1% PS), triturated in culture media, and centrifuged at 500 g for 2 minutes. The pellet was resuspended with culture media and the resulting cell suspension was plated in 75 cm² flasks coated with PLL (2 μg/mL). Cells were incubated for 2 weeks at 37° C. and 5% CO₂, with culturing media changed twice weekly. Confluency of mixed glial cultures was achieved after a culture period of 14 days in vitro.

To acquire isolated microglial cell cultures, confluent cell layers were washed with DMEM F12 and then lifted off from the 12 well plates with dilute trypsin treatment for 20 minutes (0.25% Trypsin-EDTA diluted 1:3 with DMEM/F12). Microglia remained adherent after trypsin treatment and were cultured in microglial culture media (DMEM F12 with 1% FBS and 1% PS). Mixed glial cells lifted off the substrate with trypsin treatment and were collected and subjected to two cycles of washing and centrifugation (500 g for 2 minutes), triturated in culturing media, and were deposited onto 12-well plates coated with PLL (2 μg/mL). Both mixed and isolated cultures were allowed to rest for 24 hours prior to the start of experimental procedures.

The timeline and experimental design are as follows: the cells were treated with LPS (100 ng/mL) for 6 hours (reactive) and recovered 18 hours (primed) with an effective anti-inflammatory treatment minocycline (Mino, 10 μM), dexamethasone (Dex, 10 μM), and media conditioned (CM) for 24 hours in a mixed glia culture. To fully suspend the treatments, the vehicle (Veh) contained 0.5% PEG300 in DMEM F12, which was included in Mino/Dex/CM as a final working concentration. These cells were afterwards treated with IFNγ (100 ng/mL) for 24 hours to detect whether cells were primed by their previous activation. Untreated cells (Ctr) are the surveying phenotype.

### Immunocytochemistry

Cell cultures were fixed with 5% formalin at 37° C. for 10 min and washed once with phosphate-buffered saline (PBS). Cells were then permeabilized with 0.1% Triton X-100 (TX100) in PBS and 10% Normal Horse Serum (NHS) for 1 hour. Following this, the cells were incubated overnight at 4° C. in PBS with one of the following primary antibodies plus 1% NHS: mouse anti-iNOS (1:1000), rabbit anti-Iba1 (1:1000), and chicken anti-GFAP (1:5000). Primary antibodies were then aspirated and washed 3× with PBS, followed by incubation for 1 hour at room temperature in PBS with one of the following secondary antibodies plus 1% NHS and Hoescht 33342 (1:1000): For Iba1/GFAP work, donkey anti-rabbit Alexa Fluor 488 (1:200) and goat anti-chicken Alexa Fluor 546 (1:200) were used. For iNOS, donkey anti-mouse Alex Fluor 488 (1:500) was used. For images with SX7 phages, a titre of 1010 pfu AF647 labelled phage was applied for 1 hour at 4° C. before fixation.

For adherent cultures, cells were mounted using Flourmount and 18 mm round coverslips were placed into the wells and images were acquired on a Leica DMI6000B inverted fluorescent microscope (Leica Microsystems, Wetzlar, Germany). For confocal imaging, cells were cultured on PLL-coated 18 mm glass coverslips which were fixed and subjected to immunocytochemistry as described above. Following immunocytochemistry coverslips were mounted using Fluormount onto glass slides and images acquired on a Leica TCS SPE inverted confocal microscope (Leica Microsystems).

### Molecular Analyses

Levels of the nitrite metabolite of nitric oxide (NO) were measured via the Griess reaction. Enzyme-linked immunosorbant assays (ELISA) for TNF, IL-1β, and IL-10 were performed according to the manufacturer's instructions, with modifications (R&D Systems, Minneapolis, MN). Briefly, 96-well plates were coated with the appropriate capture antibody in PBS overnight at room temperature. Plates were blocked with 1% bovine serum albumin (BSA) in PBS for 1 h at room temperature, following which samples or standards were added and incubated overnight at 4° C. Adherent antigen was detected by sequential incubation with the respective biotin-conjugated detection antibody for 1 h followed by horseradish peroxidase-conjugated streptavidin for 20 minutes (each in PBS+1% BSA). All preceding steps were separated by 3× washing phases with PBS+0.05% Tween-20 (PBS-T). Fluorescence was developed by addition of 100 μM 10-acetyl-3,7-dihydroxyphenoxazine (Amplex Red) and 0.3% H₂O₂ in PBS for 20 minutes at room temperature. Fluorescence was measured at an excitation wavelength of 540 nm and emission wavelength of 590 nm (SpectraMax M3, Molecular Devices). Cell population variation was normalized by cell count, which was obtained by post media-collection cell staining. Briefly, cell cultures were fixed with 5% formalin at 37° C. for 10 min. washed 3× with PBS, and incubated for 1 hour at room temperature in PBS with Hoechst 33342 (1:1000). See image and statistical analyses for further characterization.

### Fluorescence-Activated Cell Sorting (FACS)

Fluorescence-Activated Cell Sorting (FACS) was performed with a FACSAria III (BD Biosciences), which was used to collect populations of SX7 positive microglia and astrocytes. The immunolabelling panels were selected as following: AF647 conjugated SX7, PE-Cy7 labelled rat anti-CD11b, and PE labelled mouse anti-GLAST were used to define phage, microglial, and astrocyte populations as cell surface markers, respectively.

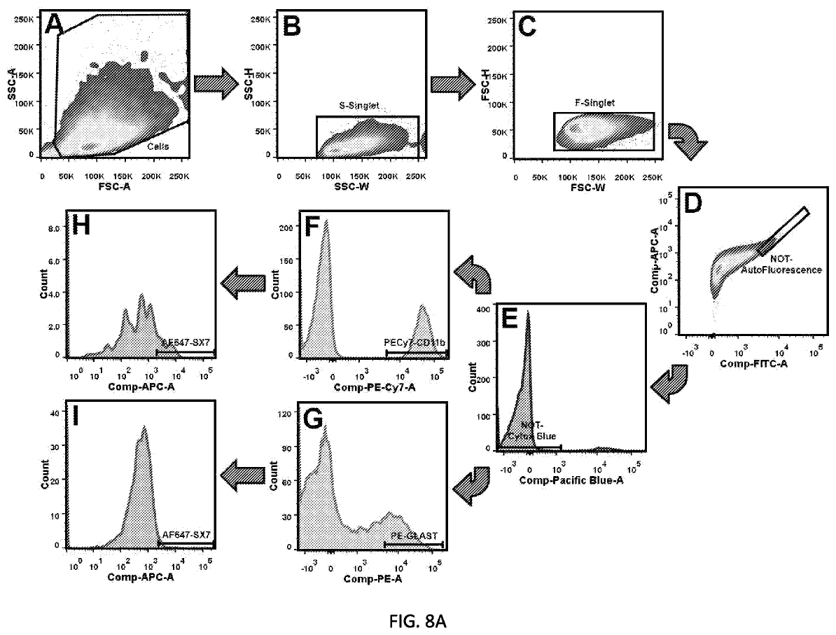

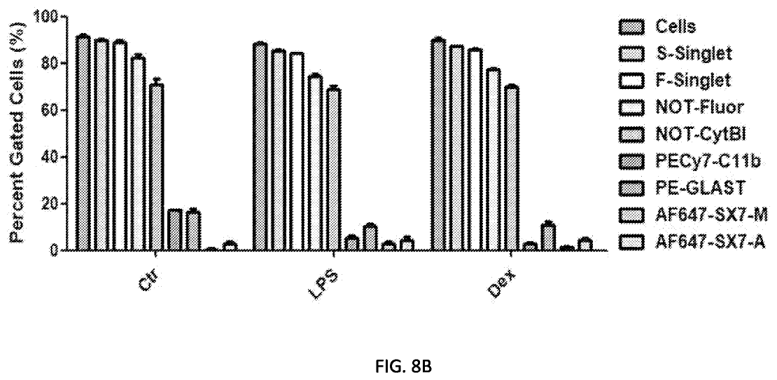

Cells were treated to the same timeline as in the culture section, in the 75 cm² flasks before being lifted off with 100% TrypLE Express (ThermoFisher), in the same conditions as the Trypsin/EDA protocol. Only Dex primed cells were used for phage incubations. Glial populations of 10 million cells/ml were incubated with 5×10¹² pfu phage, 1:1000 anti-CD11b, and 1:500 anti-GLAST in 1.5 ml PBS (10% FBS, pH 7.4) for 1 hour at 4° C. TrypLE and FBS were used in these conditions to optimize viability. The cell suspension is washed three times in PBS (10% FBS, pH 7.4), with 500 g 2 minutes centrifuge spins in between. The suspension is stained with 1:1000 Sytox Blue, inverted 3 times and can be processed by FACS. The cells are sorted based on gates shown in FIG. 8A, including pacific blue (Sytox Blue), PE, PECy5, and APC (AF647). This work was performed by the University of Alberta Flow Cytometry Facility.

### DNA Preparation and Illumina Next-Generation Sequencing (NGS)

Cells collected from FACS are spun down at 500 g, and the supernatant and pellet are collected for E. coli culture. The supernatant was used to assess content of phage lost after FACS if titres are low from the cell pellet. The following was mixed in a 5 ml FACS tube: 200 μl LB to the phage sample, 200 μl of overnight E. coli culture, and 3 ml of warm liquid agar (X-Gal/IPTG). This was cultured in a 10 cm Petri dish where titre was counted based on green coloured colony plaques compared to a standard dilution. Plated amplified phage particles were extracted by adding 3 ml PBS (pH 7.4) per plate to rock for 1 hour. Liquid was collected and centrifuged at 5,000 G for 10 min, where the supernatant was the phage solution. The solution was heated to 55° C. for 10 min (kill E. coli), then made up to a 5% PEG8000 in 0.5 M NaCl solution with the supernatant and was incubated at 4° C. overnight (precipitate phage). Centrifuge the mixture at 25,000 g for 15 min and collect pellet. Samples can be stored at 4° C. in PBS (pH 7.4) with 5% glucose for two weeks.

Polymerase Chain Reaction (PCR) was performed by reconstituting DNA samples in PBS (pH 7.4), and with in-house reverse and forward primers for SX7 so that Next-Generation Sequencing (NGS) could be done simultaneously for all samples. A Bio-Rad Thermal Cycler with a CFX96 Real Time System was used to amply the DNA. Briefly, the master mix was based on the Table 2. DNA quantity was assessed on a double comb pre-cast 0.8% agarose gel with a 50 bp ladder, SYBR1, and TAE buffer (The gel was imaged on a gel scanner with IQuant TL software (Cytiva). Using the bands, the volume was determined to add 20 ng (4 nM) of protein in a mixture in sequencing. NGS was performed using a NextSeq 550 Platform (Illumina), using the NextSeq 500/550 Mid Output Kit v2.5 150 Cycle kit (1.5 μM, 2×76 bp run with no indexing cycles). This work was done by the University of Alberta Molecular Biology Service Unit (MSBU).

TABLE 2
qPCR Formula for Sequencing

| Material | Formula | Example n = 6 | |
|---|---|---|---|
| Phusion ® HF Reaction | 6 × (n + 1) μL | 36 | μL |
| Buffer | | | |
| DNA Polymerase | 0.45 × (n + 1) μL | 3.15 | μL |
| dNTP Mix | 0.6 × (n + 1) μL | 4.20 | μL |
| SYBR1 | 1.2 × (n + 1) μL | 8.40 | μL |
| DMSO | 1.5 × (n + 1) μL | 10.50 | μL |
| NF10 forward primer | 0.6 × (n + 1) μL | 4.20 | μL |
| ‘96’ reverse primer | 0.6 × (n + 1) μL | 4.20 | μL |
| Nuclease-free water | 19 × (n + 1) μL | 133 | μL |

### Sequence Data Sorting and Analysis

Peptide sequences were determined from sequencing by strict criteria in an R script. Positive hits were determined by being present in the test set in each repeat (n=4) but not present in any other control group. For example, the test set primed microglial hits (e.g., Dex Microglia) were required to be present in four repeats while being absent from the four repeats from each control group, which included surveying microglia, surveying astrocytes, activated microglia, activated astrocytes, and primed astrocytes (e.g., Ctr Microglia, Ctr Astrocytes, LPS Microglia, LPS Astrocytes, and Dex Astrocytes respectively). Sequencing hits that were significantly enriched (p<0.05, n>3) in the test set compared to the control sets. The hit sequences were clustered based on their features described by the BLOSUM62 matrix. The clustered peptide sequences were characterized by normalized counts per million (NCPM) of each sequence in each replicate. An NCPM is how many times the peptide sequences were observed. A larger number may be interpreted as a peptide sequence that demonstrates a higher affinity for the target. Likewise, a smaller number may be indicative of a peptide sequence that has low affinity. Volcano plots were created comparing the test sets with their respective 5 control sets. Heatmaps were generated to show the normalized reads for each enriched sequence in the test set and the control sets.

Peptide Validation and in-Cell Westerns

Live mixed glia, in 48 well plates, were labelled with 100 μM peptide for 1 hour, after being treated in the timeline noted in the primary culture section (surveying, activated, Dex primed only). Peptide controls groups were swapped treatments (e.g., Surveying glia-derived peptides were also treated on activated and primed glia). Cells were washed with PBS three times, and then fixed for ten minutes to prevent peptides from washing out in subsequent immunolabelling. Specimens were immunolabelled for Iba1 and GFAP according to the protocol described in the immunocytochemistry section but incubated with the secondary donkey anti-rabbit IR Dye 680RD (1:4000). C-terminus-biotin tagged peptides were secondary labelled with 800RD Streptavidin (1:4000). A Sapphire Biomolecular Imager (Azure Biosystems) was used to scan whole plates and wells were compared with regard to relative intensity. Specimens were immunolabelled for Iba1 and GFAP, in separate samples, according to the protocol described in the immunocytochemistry section, and thereafter incubated with donkey anti-rabbit IR Dye 680RD (1:4000). C-terminus-biotin tagged peptides were labelled with 800RD Streptavidin (1:4000). Only the peptide signal that superimposed the cell labelled was considered in the data and analysis. Validated sequences were identified using protein BLAST on hosted by UniProt, within Rattus norvegicus search criteria.

### Image and Statistical Analyses

For quantification of labels, images were systematically acquired using epifluorescence microscopy on a 4×5 grid, resulting in 20 images per condition to eliminate acquisition bias. Images were quantified by automated analysis using custom written macros in ImageJ.

Overall significance was assessed using analyses of variance (ANOVA), with a Bonferroni multiple comparison post-hoc analysis between groups. A p value of ≤0.05 was considered significant, where + represent comparisons across (group-wise 2-way ANOVA) and * within (pair-wise 2-way ANOVA) Ctr, LPS, and LPS INFγ groups. Significance represented by +, where a p value of ≤0.05 was considered significant comparing (groups (1-way ANOVA) of gated cell percentages and counts, titres, and DNA quantifications.

For validating binders from in-cell western data, significances were determined by comparing clusters of experimental binders (group-wise) to both respective control treatments. For example, primed microglia's experimental group is Dex, while the control treatments are Ctr and Dex. The p values of ≤0.05, ≤0.01, and ≤0.001 were used to present significance or robust specificity of the binder and were presented as +, ++, and +++, respectively. Each reported n indicates an independent experiment from a separate culture preparation.

Results Microglial phenotypes were induced and are summarized as follows. The primary cells were pre-treated with the bacterial endotoxin lipopolysaccharide (LPS, 100 ng/mL) for 6 hours, followed by an 18-hour recovery treatment with either minocycline (Mino, 10 μM), dexamethasone (Dex, 10 μM), conditioned media (CM) from a previous 24 hr untreated glial culture, or vehicle (Veh). Treatments were washed out and cells were challenged with interferon gamma (IFNγ, 100 ng/mL) for 24 hours to probe the immune conditioning of priming by their previous activation. In order to examine the response in a more multi-cellular system comparable to in vivo, these same treatments were replicated in both isolated and mixed glia culture, consisting of astrocytes and microglia.

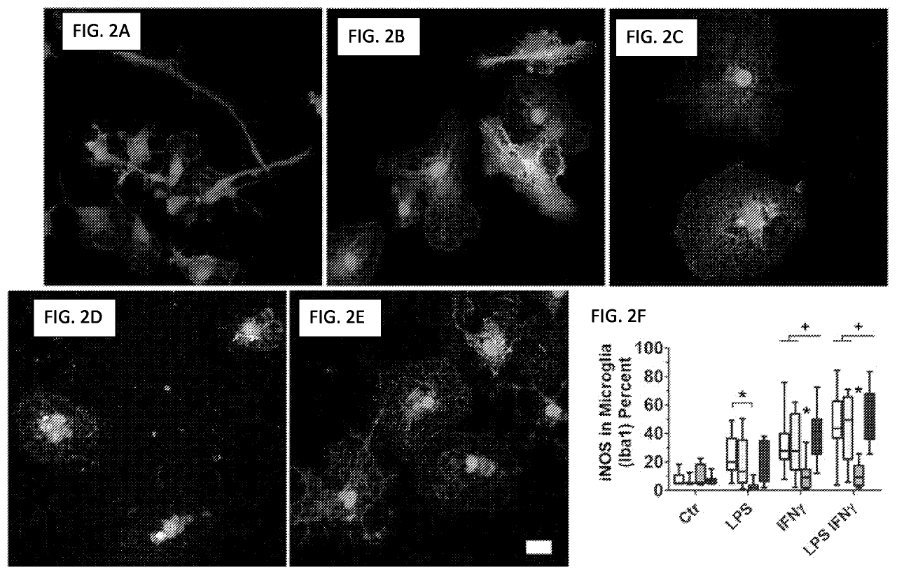

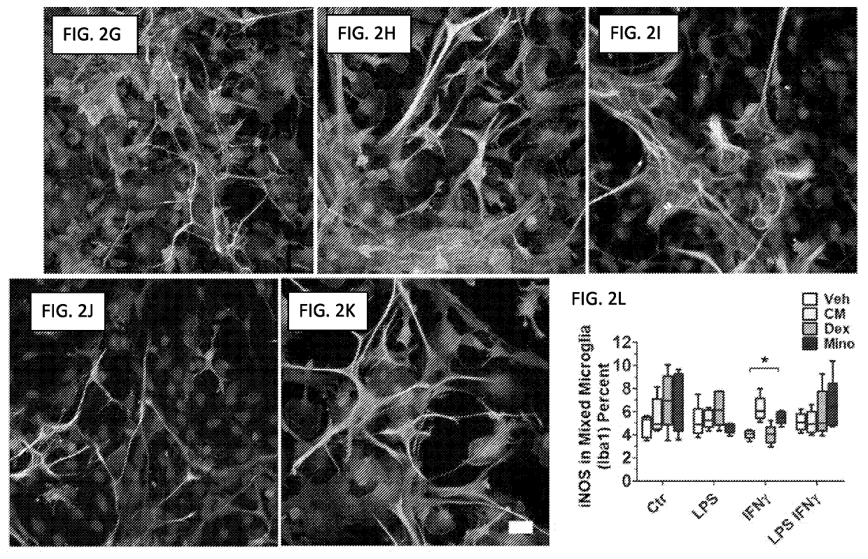

Morphology and characteristic markers of microglial function may be indicative of a primed state. Cellular morphology, observed by Iba1 and inducible nitric oxide synthase (iNOS), a typical pro-inflammatory intra-cellular protein, co-labelling in isolated microglia, demonstrates a range of morphology (FIG. 2). Isolated surveying microglia had bifurcated processes with elongated extensions. No visible iNOS expression was noted. Reactive or hyper-activated microglia presented a round amoeboid pattern of flattened cells with heterogeneous iNOS expression. LPS treatment followed by Mino and CM recovery treatments did little to change the amoeboid pattern created by LPS, however the iNOS pattern were attenuated in intensity, but not apparent frequency. In contrast, recovery treatment with Dex showed an intermediate morphology, their flattened nature reduced with a small number of large observable processes. In addition, iNOS was observed, but was less dispersed throughout the cell. The proportion of iNOS-positive cells was significantly increased by LPS (from ~5% to >20%), and only the Dex recovery treatment was able to attenuate this. In addition, activating the LPS-treated cells with IFNγ allowed for significantly more iNOS-positive microglia (from ~20% to 40%) than LPS alone, suggesting a cumulative effect on iNOS expression. Again, the Dex was the only recovery treatment able to abrogate this stimulus-induced iNOS expression.

In the mixed glial populations, the morphological features observed in isolated cells were less exaggerated with the rare expression of iNOS (FIG. 2). Surveying microglia in the control group were morphologically comparable to the isolated cells with smaller cell bodies and elongated processes. Activated/hyper-activated microglia were less amoeboid than in the isolated culture and returned to their control morphology with the Mino and Dex recovery treatments. Although GFAP-positive astrocytes tended to lose their thin processes and radial microstructures to favour a hypertrophic and/or fibrous morphology with activation treatments, regardless of recovery, the distribution of cells appears diverse in each treatment. In mixed glial cultures iNOS-positive microglia are fewer overall (~4% to 7%) suggesting the mixed cellular microenvironment limits or attenuates microglial expression of iNOS in response to inflammatory stimuli. No significant differences were noted.

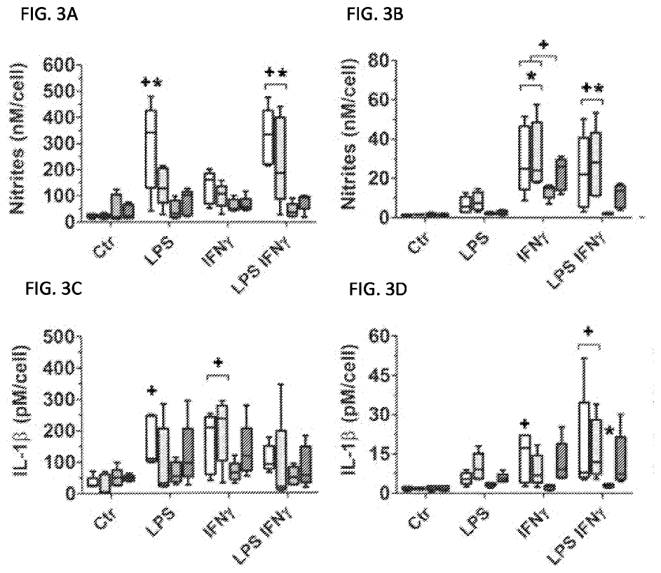

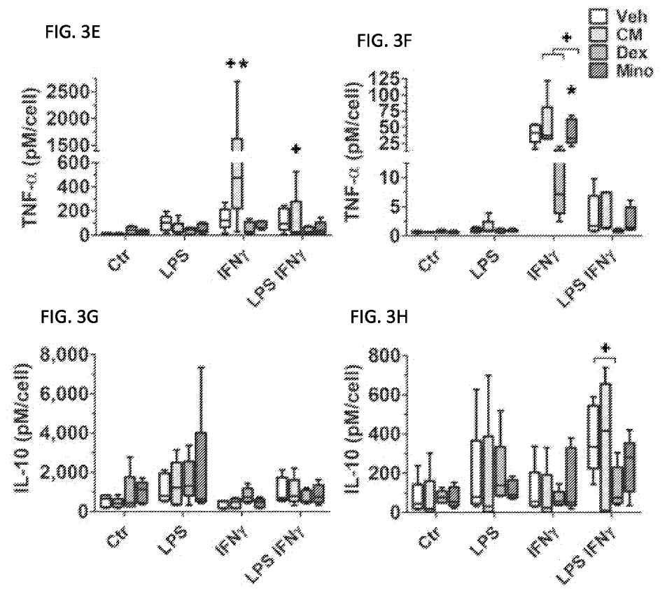

Nitrites and the major cytokines TNF and IL-1β were measured to assess pro-inflammatory secretions. Further, the cytokine IL-10 was measured to determine anti-inflammatory features (FIG. 3). Typically. LPS pre-treatment, and sequential LPS/IFNγ treatment stimulated a potent increase in pro-inflammatory factors from isolated microglial cultures. Treating isolated microglia with Mino, Dex, and CM between LPS pre-treatment and the IFNγ challenge promoted a significant reduction of TNF and nitrites, with a comparable trend of decrease in IL-1p in all activating treatments, with the exception of CM in the challenge groups. No changes were observed in IL10. The secretion of TNF and nitrites from mixed glial cultures was generally lower in the LPS treatment than the isolated microglia, indicating microglia in the mixed cultures were less potently activated, however the subsequent reduction due to recovery with Dex was more pronounced as evident from the significant pair-wise difference when cells were challenged with IFNγ. IL10 secretion was markedly higher in the LPS/IFNγ group than the control and was significantly attenuated by only the Dex recovery. This suggests a memory of activation induced by LPS pre-treatment in mixed populations even though these cells appeared to return to a baseline state of inflammatory release with only LPS. The reduction of IL10 secretion, in particular, was reduced by only Dex, suggesting that an anti-inflammatory response, in a highly reactive mixed cell microenvironment, was lost due to this drug treatment. Accordingly, Dex treatment was considered the most robust and potent model for priming. Mino and CM were able to only partially reduce pro-inflammation, while Dex was a potent anti-inflammatory agent and able to detrimentally affect anti-inflammation, trophic support, and the uptake of cellular debris. Therefore, Dex was chosen as the priming condition for phage biopanning.

The experimental design that was subsequently used for the remainder of this study on mixed glia was the following: an untreated surveying, an LPS treatment for 6 hours for a reactive population, followed by an 18-hour recovery primed treatment with of Dex. The reactive treatment was treated for 18 hours of cell culture medium. Again, these conditions were chosen as an approximation of the in vivo phenotypes using a mixed population of enriched glia in vitro. These phenotypes were incubated with an SX7 phage library and sorted with FACS (FIGS. 7-8). To demonstrate phage binding before and after FACS, confocal microscopy was performed on glia in culture and in suspension after the sort (FIG. 5A-C). Phage particles can be seen attached to glia in surveying, reactive, and primed conditions, where glial morphology was comparable as shown previously (FIG. 2). Sorted cells were mostly spherical, and indistinguishable between groups, but phage particles binding to sorted cells was clearly evident. Although some particles may be undergoing a type of endocytosis, which is expected from immune cells, many particles are clearly not doing so in these images.

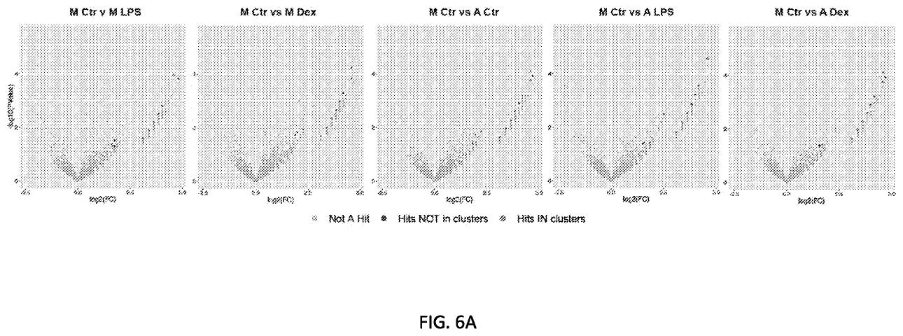

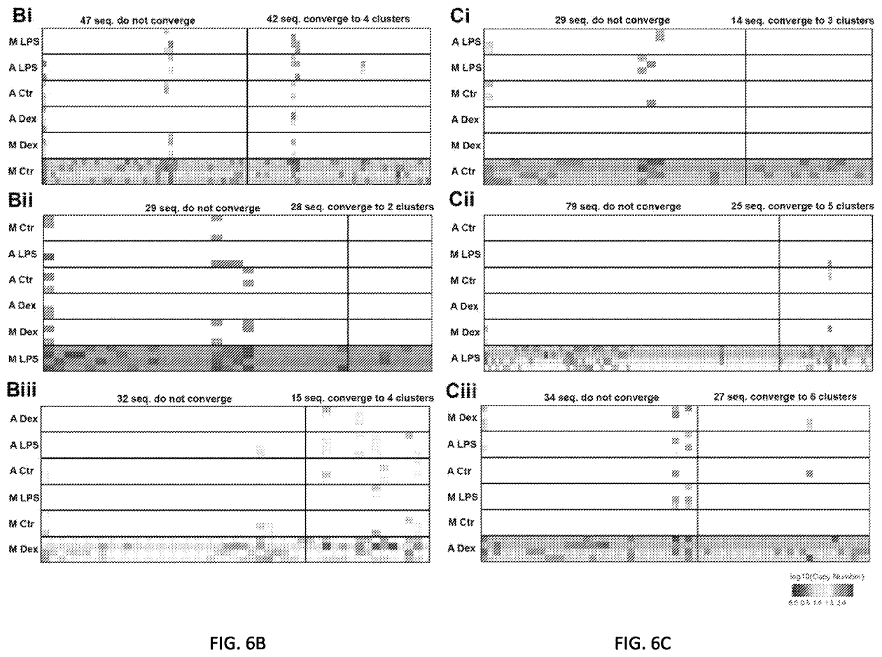

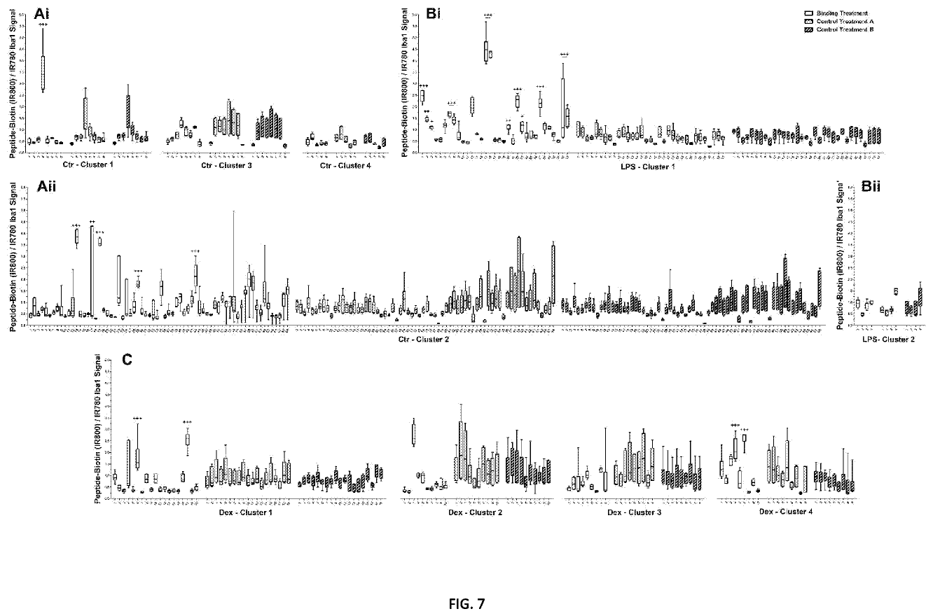

Due to the large number of putative targets detected during sequencing, differential analysis was performed to identify sequences present in the experimental group but absent in the respective control groups, and in silico enrichment techniques were employed to cluster peptide features using the BLOSUM62 matrix. A representative summary of this data is presented in FIG. 6A. Volcano plots were used to characterize clustering and heat maps to represent quantification of sequences by normalized counts per million (NCPM) (FIG. 6). Comparison of NCPM on the volcano plots display significantly enriched peptide sequences (i.e., was observed with a larger NCPM with higher significance) in the upper right quadrant for each indicated pair-wise comparison (FIG. 6A). Sequence clustering is summarized in a series of heat maps (FIG. 6B-C), where enriched sequences are indicated against all experimental conditions, with colour temperature indicating NCPM (log₁₀(copy number)) for enriched sequences in both microglia and astrocytes. The number of sequence clusters varied from 2 to 6, where reactive microglia proved to have the lowest clusters (2) but consistently had the highest relative enrichment as NCPM. Astrocytes tended to show the opposite, where higher intensity with lower (3) clustering occurred in the surveying group, and reactive showed increases in clustering (5-6).

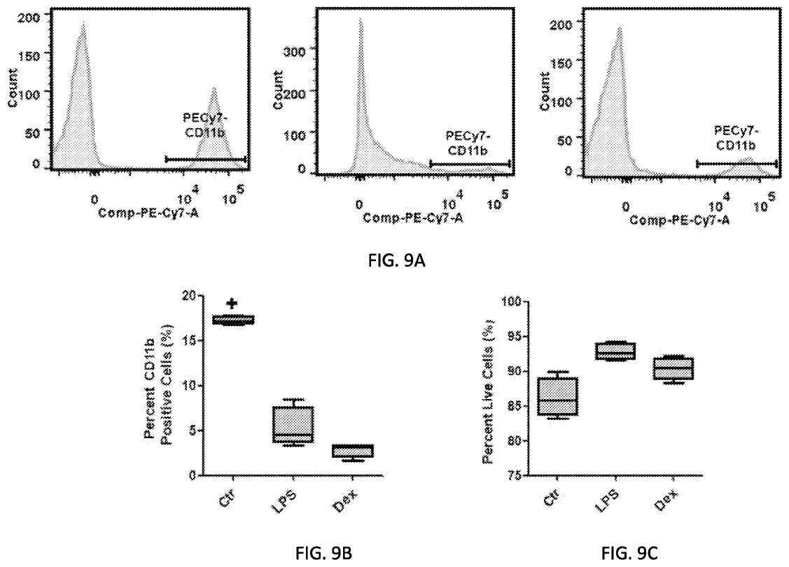

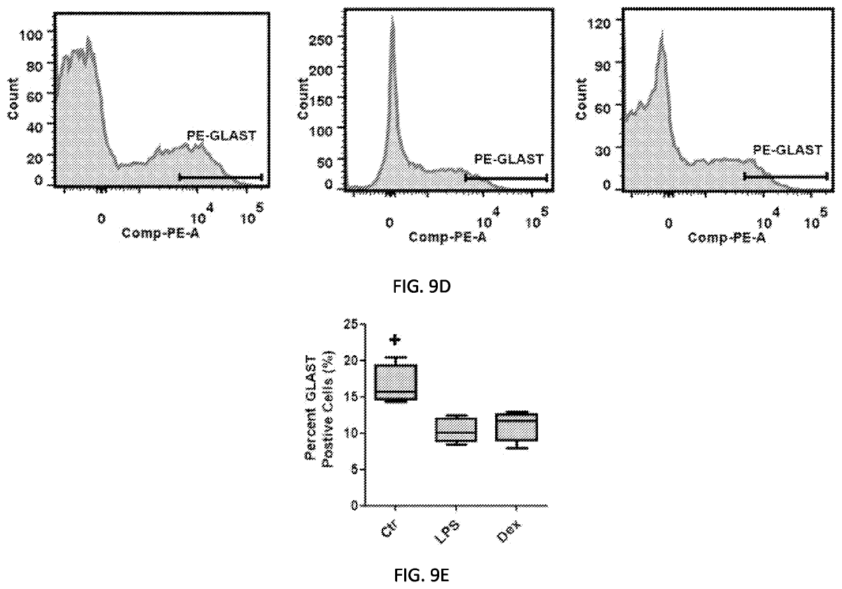

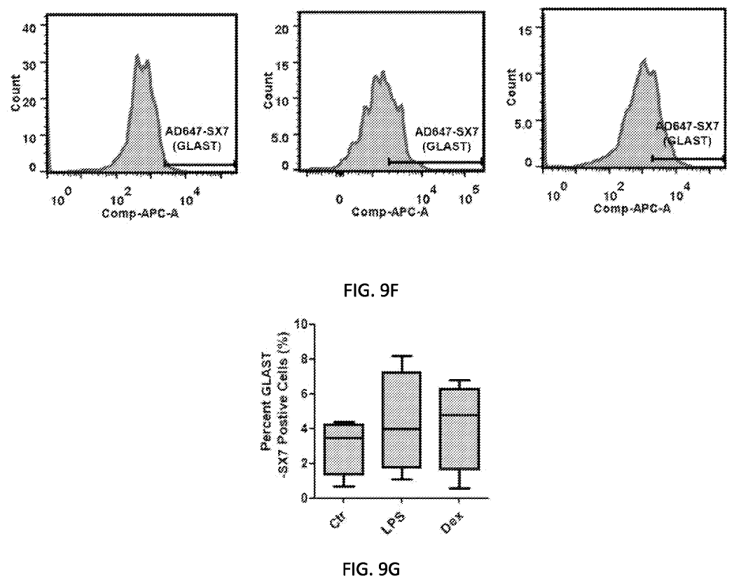

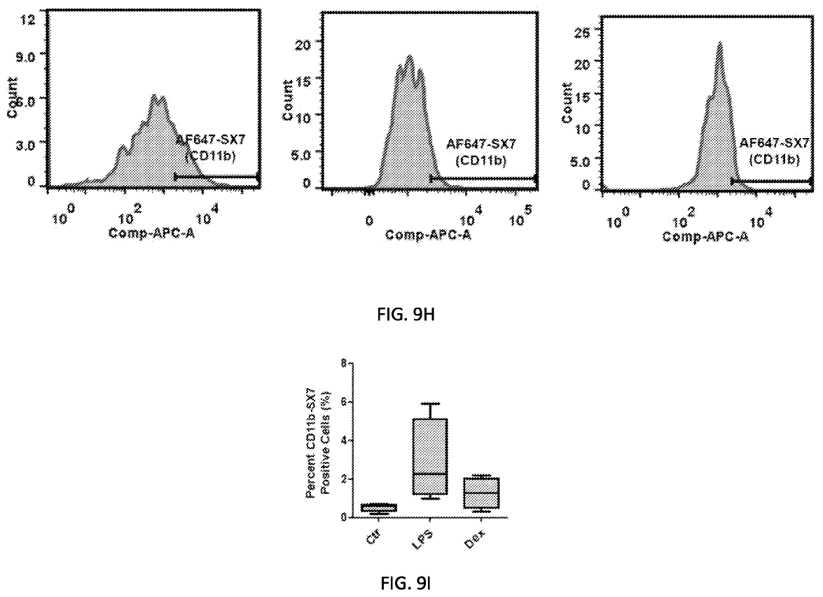

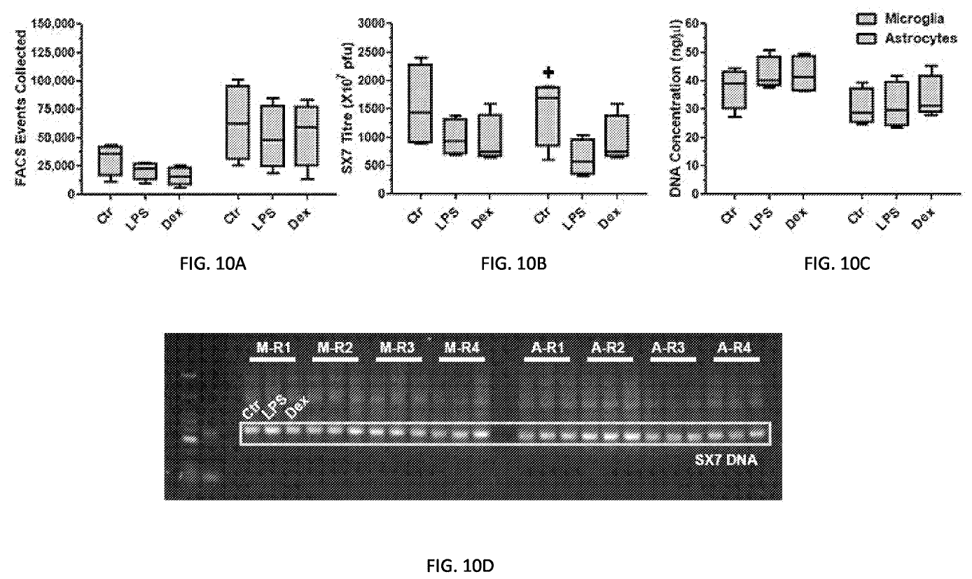

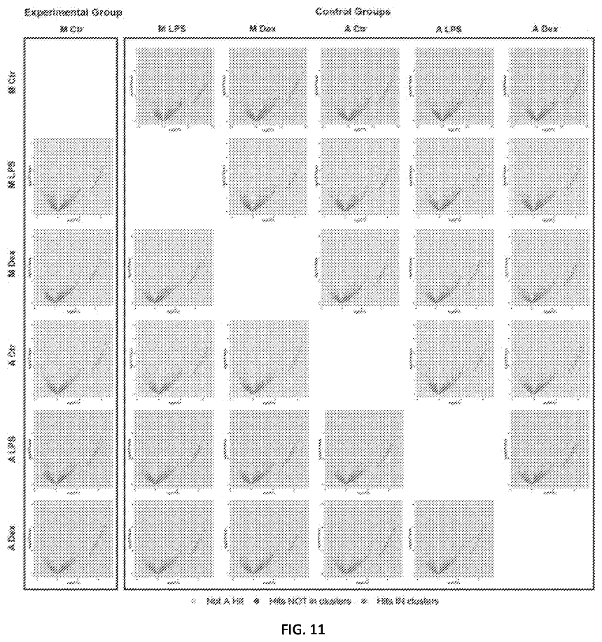

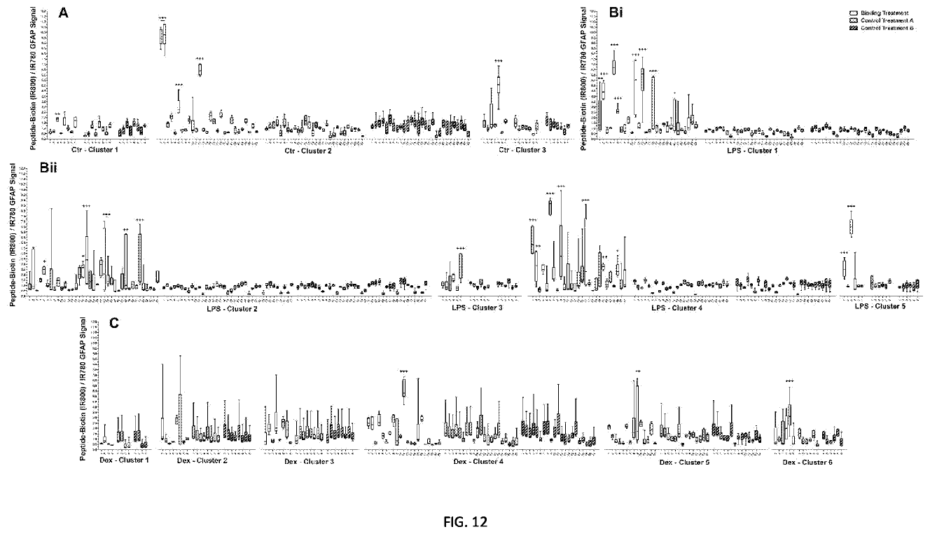

Despite the strong identification of phage-displayed sequences enriched from the analysis, steps were required to eliminate possible artefacts due to interactions with the viral coat, structural interactions at the attachment site of peptide attachment, or any other interactions with the phage itself. This is of particular concern given that microglia and astrocytes can express pattern recognition receptors that have broad specificity for viral and bacterial antigens. To address this peptide adhesion was assessed in the absence of phage using synthetic peptides on mixed glia cultures after phenotypic treatments, and binding was assessed using an in-cell western (FIG. 7). Peptide binding was quantified by the average intensity ratio of peptide relative to either a microglia- or astrocyte-specific marker. To confirm that peptide binding was specific to each specific phenotypic treatment, peptides were validated across treatment groups and only those peptides that presented significant binding for the appropriate phenotype overreach control were included (e.g., significant binding of reactive peptides in the reactive condition relative to surveying or primed). All other peptides are presented in FIG. 9 and FIG. 10. Validated peptides for each condition and the complete peptide sequences are summarized in Table 3. Following peptide binding validation 58 positive binders remained, with the highest number of peptides found in reactive microglia and astrocytes. Based on peptide binding intensity, astrocytes tended to have more potent binders than microglia. This parallels the number of cell events collected from FACS and may relate to the larger cell surface area available for peptide interaction on astrocytes compared to the smaller microglial cells. Despite being relatively low in clustering (2) and un-validated sequences (37/173), reactive microglia had many significant binders (14/24) although the number of potent binders might be comparable (greater than 2-fold binding ratio to the controls). In contrast, reactive astrocytes were relatively high in clustering (5), pre-screened binding (192/296), and validated binding (25/34). A small number of sequences were specific for the primed state of both glial cell types. Most sequences (47/58) are either in (*), or close to (−) the clusters. Non-specific binding, noted as progressively higher signals in controls, tends to trend higher as peptides further from the clustering, especially in surveying microglia (FIG. 12A).

Since phage display is a deliberately target naïve technique, the possible sequence origins of the discovered peptides were not determined, with the limited exception of the BLOSUM62 clustering analysis. After peptide binding validation, the shorter list of peptides was analyzed using the basic alignment search tool (BLAST) to speculate on the nature of these peptides based on sequence similarity to published protein sequences. The complete results are presented in Tables 6-11, with a representative set of candidates summarized in Tables 4 and 5. The results of the BLAST included secreted or extra-cellular proteins, which are expected in cell-protein interactions, but also included cell surface receptors and intra-cellular proteins. Glia can be involved in vesicle trafficking, mitotic regulation of other cells, synaptic pruning, activation via intra-cellular components of necrotic cell death, among many other unique cell behaviours. As such, glial peptide binding could originate from many unanticipated source proteins.

TABLE 3
Summary of Validated Peptide Sequences Adherent to Glial Phenotypes

| Cell | | | | | | | Signifi- |
|---|---|---|---|---|---|---|---|
| Type | Phenotype | Clusters | Sequences | Binders | Code | Sequence | cance |
| Microglia | Surveying | 4 | 89 | 6 | M−C1 | SSLVLADS | +++ |
| (M) | (Ctr, −) | | | | −4− | (SEQ ID | +++ |
| | | | | | M−C2 | NO: 1) | ++ |
| | | | | | −13* | SSNSVVGI | +++ |
| | | | | | M−C2 | (SEQ ID | +++ |
| | | | | | −17* | NO: 2) | +++ |
| | | | | | M−C2 | SMSTYVPS | |
| | | | | | −19* | (SEQ ID | |
| | | | | | M−C2 | NO: 3) | |
| | | | | | −24 | STHTTSVL | |
| | | | | | M−C2 | (SEQ ID | |
| | | | | | −44− | NO: 4) | |
| | | | | | | SSSSLLHY | |
| | | | | | | (SEQ ID | |
| | | | | | | NO: 5) | |
| | | | | | | SHSYAGV | |
| | | | | | | L (SEQ ID | |
| | | | | | | NO: 6) | |
| | Activated | 2 | 37 | 14 | M+C1 | SSLTTLTA | +++ |
| | (LPS, +) | | | | −1* | (SEQ ID | ++ |
| | | | | | M+C1 | NO: 7) | + |
| | | | | | −2* | SLASAVSN | +++ |
| | | | | | M+C1 | (SEQ ID | +++ |
| | | | | | −3* | NO: 8) | +++ |
| | | | | | M+C1 | SSALTVSH | +++ |
| | | | | | −7− | (SEQ ID | +++ |
| | | | | | M+C1 | NO: 9) | ++ |
| | | | | | −8− | SVAVEAH | +++ |
| | | | | | M+C1 | H (SEQ ID | + |
| | | | | | −12− | NO: 10) | +++ |
| | | | | | M+C1 | SHIGTVTG | +++ |
| | | | | | −15− | (SEQ ID | +++ |
| | | | | | M+C1 | NO: 11) | |
| | | | | | −16− | SVMTINNT | |
| | | | | | M+C1 | (SEQ ID | |
| | | | | | −20− | NO: 12) | |
| | | | | | M+C1 | SAQTLDRS | |
| | | | | | −22− | (SEQ ID | |
| | | | | | M+C1 | NO: 13) | |
| | | | | | −23− | SLIEWYSA | |
| | | | | | M+C1 | (SEQ ID | |
| | | | | | −27− | NO: 14) | |
| | | | | | M+C1 | SYMNNSV | |
| | | | | | −32− | F (SEQ ID | |
| | | | | | M+C1 | NO: 15) | |
| | | | | | −33− | SDTPTSPG | |
| | | | | | | (SEQ ID | |
| | | | | | | NO: 16) | |
| | | | | | | SDPSQFPL | |
| | | | | | | (SEQ ID | |
| | | | | | | NO: 17) | |
| | | | | | | SHLSYTM | |
| | | | | | | W (SEQ ID | |
| | | | | | | NO: 18) | |
| | | | | | | SGNEDRY | |
| | | | | | | P (SEQ ID | |
| | | | | | | NO: 19) | |
| | | | | | | SHDDIPHL | |
| | | | | | | (SEQ ID | |
| | | | | | | NO: 20) | |
| | Primed | 4 | 47 | 4 | MDC1 | SVDSNNE | +++ |
| | (Dex, D) | | | | −6− | H (SEQ ID | +++ |
| | | | | | MDC1 | NO: 21) | +++ |
| | | | | | −17− | SFGFMNL | +++ |
| | | | | | MDC4 | H (SEQ ID | |
| | | | | | −4− | NO: 22) | |
| | | | | | MDC4 | STHVQNW | |
| | | | | | −6− | L (SEQ ID | |
| | | | | | | NO: 23) | |
| | | | | | | SNIMHSLN | |
| | | | | | | (SEQ ID | |
| | | | | | | NO: 24) | |
| Astro- | Surveying | 3 | 43 | 6 | A− | SNVIDPAN | ++ |
| cytes (A) | (Ctr, −) | | | | C1−3 | (SEQ ID | +++ |
| | | | | | A− | NO: 25) | +++ |
| | | | | | C2−1* | SGNSTSNA | +++ |
| | | | | | A− | (SEQ ID | +++ |
| | | | | | C2−2* | NO: 26) | +++ |
| | | | | | A− | SSTVAND | |
| | | | | | C2−6 | G (SEQ ID | |
| | | | | | A− | NO: 27) | |
| | | | | | C2−12− | SPNTNTLA | |
| | | | | | A− | (SEQ ID | |
| | | | | | C3−5− | NO: 28) | |
| | | | | | | SVFNVNN | |
| | | | | | | T (SEQ ID | |
| | | | | | | NO: 29) | |
| | | | | | | SAYLDSLN | |
| | | | | | | (SEQ ID | |
| | | | | | | NO: 30) | |
| | Activated | 5 | 192 | 25 | A+C1− | SVSTTGVR | ++ |
| | (LPS, +) | | | | 1* | (SEQ ID | +++ |
| | | | | | A+ | NO: 31) | +++ |
| | | | | | C1−2* | SFSNIAIR | +++ |
| | | | | | A+ | (SEQ ID | +++ |
| | | | | | C1−5 | NO: 32) | +++ |
| | | | | | A+ | SLSAVDVS | |
| | | | | | C1−6 | (SEQ ID | + |
| | | | | | A+ | NO: 33) | + |
| | | | | | C1−11− | SLLTTGPV | + |
| | | | | | A+ | (SEQ ID | +++ |
| | | | | | C1−13− | NO: 34) | +++ |
| | | | | | A+ | SGSMIAMP | +++ |
| | | | | | C1−16− | (SEQ ID | ++ |
| | | | | | A+ | NO: 35) | +++ |
| | | | | | C1−22− | SSAGMHV | +++ |
| | | | | | A+ | L (SEQ ID | +++ |
| | | | | | C2−5 | NO: 36) | ++ |
| | | | | | A+ | SHNSQSIP | +++ |
| | | | | | C2−16− | (SEQ ID | +++ |
| | | | | | A+ | NO: 37) | +++ |
| | | | | | C2−17− | SLGMTYG | ++ |
| | | | | | A+ | H (SEQ ID | + |
| | | | | | C2−21− | NO: 38) | +++ |
| | | | | | A+ | SMPDASLS | +++ |
| | | | | | C2−22− | (SEQ ID | |
| | | | | | | NO: 39) | |
| | | | | | A+ | SQQTDSYP | |
| | | | | | C2−28− | (SEQ ID | |
| | | | | | A+ | NO: 40) | |
| | | | | | C2−32− | SASVHPLA | |
| | | | | | A+ | (SEQ ID | |
| | | | | | C3−6− | NO: 41) | |
| | | | | | A+ | SWTAYMS | |
| | | | | | C4−1* | S (SEQ ID | |
| | | | | | A+ | NO: 42) | |
| | | | | | C4−2* | SPQLTRNV | |
| | | | | | A+ | (SEQ ID | |
| | | | | | C4−6 | NO: 43) | |
| | | | | | A+ | SHAHSLSV | |
| | | | | | C4−9 | (SEQ ID | |
| | | | | | A+ | NO: 44) | |
| | | | | | C4−16 | SNPYSPSL | |
| | | | | | A+ | (SEQ ID | |
| | | | | | C4−21 | NO: 45) | |
| | | | | | A+ | SYLPDEFT | |
| | | | | | C4−25 | (SEQ ID | |
| | | | | | A+ | NO: 46) | |
| | | | | | C5−1* | STIQFTPQ | |
| | | | | | A+ | (SEQ ID | |
| | | | | | C5−3 | NO: 47) | |
| | | | | | | STLSFTRM | |
| | | | | | | (SEQ ID | |
| | | | | | | NO: 48) | |
| | | | | | | STWSYPRI | |
| | | | | | | (SEQ ID | |
| | | | | | | NO: 49) | |
| | | | | | | SPLSFQAT | |
| | | | | | | (SEQ ID | |
| | | | | | | NO: 50) | |
| | | | | | | SNFPSLSV | |
| | | | | | | (SEQ ID | |
| | | | | | | NO: 51) | |
| | | | | | | SNKMDNT | |
| | | | | | | L (SEQ ID | |
| | | | | | | NO: 52) | |
| | | | | | | SPIRGYTD | |
| | | | | | | (SEQ ID | |
| | | | | | | NO: 53) | |
| | | | | | | SVDDYFT | |
| | | | | | | R (SEQ ID | |
| | | | | | | NO: 54) | |
| | | | | | | SIDYMYA | |
| | | | | | | R (SEQ ID | |
| | | | | | | NO: 55) | |
| | Primed | 6 | 61 | 3 | AD | STTVNTIA | +++ |
| | (Dex, D) | | | | C4−11− | (SEQ ID | ++ |
| | | | | | AD | NO: 56) | +++ |
| | | | | | C5−9− | STYHTNSS | |
| | | | | | AD | (SEQ ID | |
| | | | | | C6−5− | NO: 57) | |
| | | | | | | SGSSFTMI | |
| | | | | | | (SEQ ID | |
| | | | | | | NO: 58) | |

Postscripts * and − represent core clustered and un-clustered, but close, sequences, respectively, based on features described by the BLOSUM64 matrix.

TABLE 4
Summary of BLAST Identities for Microglial-Derived Peptides and Possible Function

| Pheno- | | Pep- | | |
|---|---|---|---|---|
| type | Code | tide | Origin Protein | Possible Role |
| Survey- | M−C1− | SSLV | Hemicentin 1 | Neuronal differentiation, pinched synaptosomes and |
| ing | 4− | LADS | | microglial synaptic pruning |
| (Ctr, | | (SEQ | Olfactory receptor 34 | Microglial regulation and survival olfactory neurons |
| −) | | ID | and 14C36-like | |
| | | NO: | | |
| | | 1) | | |
| | M−C2− | SSNS | Olfactory receptor | Microglial regulation and survival olfactory neurons |
| | 13* | VVGI | | |
| | | (SEQ | | |
| | | ID | | |
| | | NO: | | |
| | | 20 | | |
| | M−C2− | SMST | Thyroid hormone | Glial mediated thyroid function and dysfunction |
| | 17* | YVPS | receptor alpha | |
| | | (SEQ | Chorion-specific | Hormonal signalling to regulate microglia |
| | | ID | transcription factor | |
| | | NO: | GCMa | |
| | | 3) | TAP binding protein | Mediates interaction between newly assembled MHC. |
| | | | | transport of antigenic peptides across the |
| | | | | endoplasmic reticulum membrane, promotes neuro- |
| | | | | protection |
| | M−C2− | STHT | Rho family-interacting | Microglia-promoted neuroprotection |
| | 19* | TSVL | cell polarization | |
| | | (SEQ | regulator 1 | |
| | | ID | | |
| | | NO: | | |
| | | 4) | | |
| | M−C2− | SSSS | Follistatin-related | Neuroprotective and immunoregulatory effects on pro- |
| | 24 | LLHY | protein 1 | inflammatory microglial activation |
| | | (SEQ | Trace amine-associated | Regulation and signalling in olfactory neurons |
| | | ID | receptor 7g | |
| | | NO: | Interleukin 12 receptor | T-cell differentiation or stimulation, from |
| | | 5) | subunit beta 2 | dendritic cells, microglia, and neutrophils |
| | | | Olfactory receptor 869 | Microglial regulation and survival olfactory neurons |
| | | | Teneurin-2 | Axonal guidance, synapses, dendritic spines, |
| | | | | filopodium, growth cones, and synaptosomes |
| | | | Insulin receptor | Hormonal signalling in neurotrauma and neuro- |
| | | | substrate 2 | degeneration |
| | | | Extended synaptotagmin | Neurotransmitter release at presynaptic nerve |
| | | | 2 | terminals |
| | | | G protein-regulated | Survival and neuritogenesis |
| | | | inducer of neurite | |
| | | | outgrowth 2 | |
| | M−C2− | SHSY | CD177 antigen-like | Neutrophil activation marker |
| | 44− | AGVL | Gamma-crystallin A | Microglial regulation and survival of retinal |
| | | (SEQ | | ganglia |
| | | ID | | |
| | | NO: | | |
| | | 6) | | |
| Acti- | M+C1− | SSLT | Synergin gamma | Adapter proteins involved in endocytic and endosomal |
| vated | 1* | TLTA | | machinery |
| (LPS, | | (SEQ | Receptor-type tyrosine- | Promote T cell activation |
| +) | | ID | protein phosphatase C | |
| | | NO: | Cadherin-2 | Neuronal connectivity, calcium dependent cell |
| | | 7) | | adhesion, and role in synapses memory plasticity |
| | M+C1− | SLAS | Stonin-2 | Adapter proteins involved in endocytic and endosomal |
| | 2* | AVSN | | machinery |
| | | (SEQ | Catenin, delta 1, | Neuronal connectivity, calcium dependent cell |
| | | ID | isoform CRA_a | adhesion, and role in synapses memory plasticity |
| | | NO: | | |
| | | 8) | | |
| | M+C1− | SHIG | Protocadherin beta 21 | Neuronal connectivity, calcium dependent cell |
| | 8− | TVTG | | adhesion, and role in synapses memory plasticity |
| | | (SEQ | | |
| | | ID | | |
| | | NO: | | |
| | | 11) | | |
| | M+C1− | SVMT | BRD4-interacting | Glial proliferation |
| | 12− | TNNT | chromatin-remodeling | |
| | | (SEQ | complex- associated | |
| | | ID | protein-like | |
| | | NO: | Oxysterol-binding | Cholesterol binding, uptake, and phagocytosis |
| | | 12) | protein | |
| | M+C1− | SLIE | Gap junction alpha-1 | Cross-communication with microglia and astrocytes in |
| | 16− | WYSA | protein | synaptic maintenance, astrocytes in glaucomatous |
| | | (SEQ | | neurodegeneration |
| | | ID | Connexin-43 | Cross-communication with eosinophils and |
| | | NO: | | neuroinflammation with microglia and astrocytes, |
| | | 14) | | astrogliosis |
| | M+C1− | SYMN | Cyclin-L1, L2, and H | Phosphorylation in neuroinflammation, tumor |
| | 20− | NSVE | | promoting inflammation, and chronic inflammation |
| | | (SEQ | Phosphatidylinositol-3- | Lipid binding and uptake |
| | | ID | phosphate phosphatase | |
| | | NO: | C-Maf-inducing protein | Phosphorylation in inflammation |
| | | 15) | Dynein axonemal heavy | Retrograde fast axonal transport |
| | | | chain 7 | |
| | M+C1 | SDTP | Neuronal migration | Adhesion and migration, present in amyloid plaque |
| | 22 | TSPG | protein doublecortin | resident activated microglia |
| | | (SEQ | Neural cell adhesion | Adhesion and migration of neutrophils and microglia |
| | | ID | molecule 1 | |
| | | NO: | Mitogen-activated | Glial proliferation |
| | | 16) | protein kinase | |
| | | | kinasekinase 10 | |
| | M+C1 | SGNE | Matrix metallopeptidase | Re-organizing ECM material during neuroinflammation |
| | 32 | DRYP | 21 | and gliomas |
| | | (SEQ | Protocadherin Fat 2 | Neuronal connectivity, calcium dependent cell |
| | | ID | | adhesion, and role in synapses memory plasticity |
| | | NO: | Extracellular calcium- | Regulates ionic homeostasis and is present in most |
| | | 19) | sensing receptor | major glia |
| | M+CI1 | SHDD | Interferon-induced | Regulate innate immunity |
| | 33 | IPHL | protein 44-like | |
| | | (SEQ | | |
| | | ID | | |
| | | NO: | | |
| | | 20) | | |
| | MDCI1 | SVDS | Eukaryotic translation | Nerve injury-induced alternations in gene expression |
| | 6 | NNEH | initiation factor 4 | during mitosis and is associated with nociceptive |
| | | (SEQ | gamma, 1 | hypersensitivity |
| | | ID | Condensin complex | Chromosome assembly and segregation during cell |
| | | NO: | subunit 1 | division, damage linked to microglia in neuro- |
| | | 21) | | degenerative and psychiatric diseases, |
| | | | Xylosyltransferase | Biosynthesis of chondroitin sulfate and dermatan |
| | | | | sulfate proteoglycans in glial scar |
| | | | Laminin, alpha 1 and | ECM binding of activated microglia |
| | | | subunit alpha 1 | |
| | | | Protocadherin-16 | Neuronal connectivity, calcium dependent cell |
| | | | | adhesion, and role in synapses memory plasticity |
| | | | Thyroid hormone | Glial mediated thyroid function and dysfunction |
| | | | receptor interactor 11 | |
| | MDC1 | SFGF | EF-hand calcium-binding | In Iba1, a target used extensively in immuno- |
| | −17− | MNLH | domain 7 | labelling |
| | | (SEQ | Sema domain, immuno- | Axonal guidance protein, has a role in neuro- |
| | | ID | globulin domain (Ig), | inflammation processes, in axonal growth cone |
| | | NO: | short basic domain, | guidance molecules, and are cues to deflect axons |
| | | 22) | secreted, (Semaphorin) | from inappropriate regions |
| | | | 3E (Predicted) | |
| | | | Synaptophysin | In presynaptic vesicles of neurons and microglia |
| | | | | were able to be focally inhibited in a NO synthase- |
| | | | | dependent manner |
| | MDC4 | STHV | Dynein, axonemal, heavy | Retrograde fast axonal transport |
| | −4− | QNWL | chain 11 | |
| | | (SEQ | | |
| | | ID | | |
| | | NO: | | |
| | | 23) | | |
| | MDC$ | SNIM | Receptor-type tyrosine- | Microglial role in oligodendrocyte populations, |
| | −6− | HSLN | protein phosphatase | responsible for creating white matter |
| | | (SEQ | | |
| | | ID | | |
| | | NO: | zeta | |
| | | 24) | | |

Postscripts * and − represent core clustered and un-clustered, but close, sequences, respectively, based on features described by the BLOSUM64 matrix.
Colours represent residues that are polar, non-polar, negatively charged, and positively charged.
Acronyms: glial cells missing (GCM), extracellular matrix (ECM), cluster differentiation (CD).

TABLE 5
Summary of BLAST Identities for Astroglial-Derived Peptides and Possible Function

| Pheno- | | Pep- | | |
|---|---|---|---|---|
| type | Code | tide | Origin Protein | Possible Role |
| Survey- | A− | SNVI | Netrin receptor UNC5B | Attenuates brain injury and regulates microglial |
| ing | C1− | DPAN | and UNC5 | accumulation along axons which supports the neuronal |
| (Ctr, | 3 | (SEQ | | survival |
| −) | | ID | | |
| | | NO: | | |
| | | 25) | | |
| | A− | SGNS | Fibronectin type III | Neurite outgrowth, neuron migration, adhesion, and is |
| | C2− | TSNA | domain- containing 3C1 | secreted by reactive astrocytes |
| | 1* | (SEQ | | |
| | | ID | | |
| | | NO: | | |
| | | 26) | | |
| | A− | SSTV | Neuropilin and tolloid- | Neuronal regulation and development, retinal angiogenesis, |
| | C2− | ANDG | like protein 1 and 2 | and microglial-mediated brain vascularisation as a cellular |
| | 2* | (SEQ | | chaperone for vessel fusion |
| | | ID | | |
| | | NO: | | |
| | | 27) | | |
| | A− | SPNT | Condensin-2 complex | Healthy gene expression in new neuronal cell division in |
| | C2− | NTLA | subunit D3 | the developing and adult hippocampal brain |
| | 6 | (SEQ | Mast cell protease 4 | Activator for astrocytes where glial cells and neurons |
| | | ID | | interact with mast cells and accelerate neuroinflammation |
| | | NO: | | |
| | | 28) | | |
| | A− | SVFN | cGMP-gated cation channel | Cation-neuronal depolarization |
| | C2− | VNNT | alpha-1 | |
| | 12− | (SEQ | Cyclic nucleotide-gated | Cation-neuronal depolarization |
| | | ID | cation channel 1 | |
| | | NO: | Probable G-protein | G-protein astrocyte-neuron interaction |
| | | 29) | coupled receptor 158 | |
| | A | SAYL | ATP-binding cassette | Metabolic ATP regulators, in the blood brain barrier |
| | C3 | DSLN | subfamily A member 2 and | endothelium with astrocyte foot processes |
| | 5 | (SEQ | 12, and subfamily E | |
| | | ID | member 1 | |
| | | NO: | Exopolyphosphatase PRUNE1 | Neurogenesis by regulating microtubule polymerization |
| | | 30) | CUB and Sushi multiple | Schizophrenia and excessive astrocyte-mediated synaptic |
| | | | domains 2 | pruning |
| | | | Adenylate kinase | Lipid transport in neuroinflammation |
| | | | isoenzyme 1 | |
| | | | Nucleoside-diphosphate | Lipid transport in neuroinflammation |
| | | | kinase | |
| | | | NACHT, leucine rich | Inflammasome present in astrocytes, which may be a key |
| | | | repeat and PYD | mediator in the recovery after severe brain injury |
| | | | containing 5 (Predicted) | |
| | | | (Rattus norvegicus) | |
| Acti- | A+ | SESN | Early growth response | Cell proliferation and apoptosis regulation via growth |
| vated | C1 | IAIR | protein | factors, cytokines, and stress signals. Synaptic plasticity |
| (LPS, | 2* | (SEQ | | and neuronal activity in both physiological and pathological |
| +) | | ID | | conditions, especially neuropsychiatric disorders |
| | | NO: | | |
| | | 32) | | |
| | A+ | SLSA | Olfactory receptor | Sensory circuit processing |
| | C1 | VDVS | Mucin 5B, oligomeric | Glycopolymer that has been shown to direct electrolocation |
| | 5 | (SEQ | mucus/gel-forming | and neuronal activation |
| | | ID | | |
| | | NO: | | |
| | | 33) | | |
| | A+ | SLLT | Mucin 5B, oligomeric | Glycopolymer that has been shown to direct electrolocation |
| | C1 | TGPV | mucus/gel-forming | and neuronal activation |
| | 6 | (SEQ | LIM domain kinase 2 and | In a subpopulation of protoplasmic adult glial cells during |
| | | ID | isoform CRA_e | CNS development |
| | | NO: | | |
| | | 34) | | |
| | A+ | SGSM | Dedicator of cytokinesis | Hippocampal development of neurons, where Dock7- |
| | C1 | IAMP | 7 | abundant singular neurites form into polarized axons |
| | 11 | (SEQ | High affinity cationic | Neuroinflammation and immunity physiology in neurons |
| | | ID | amino acid transporter 1 | and astrocytes where loss is linked to neurodegenerative |
| | | NO: | | disorders |
| | | 35) | Ecotropic retrovirus | Subdetectable astrocyte expression of exogenous or |
| | | | receptor | endogenous retroviruses linked to neurodegenerative |
| | | | | disease |
| | | | Vomeronasal type-1 | Production of pheromones present in neurons, microglia, |
| | | | receptor | and astrocytes |
| | A+ | SHNS | FAT atypical cadherin 4 | Developing neuronal circuits and are mediators for glial |
| | C1 | QSIP | | morphology |
| | 16 | (SEQ | | |
| | | ID | | |
| | | NO: | | |
| | | 37) | | |
| | A+ | SLGM | Vomeronasal 2 receptor, | Production of pheromones present in neurons, microglia, |
| | C1 | TYGH | 79 | and astrocytes |
| | 22 | (SEQ | | |
| | | ID | | |
| | | NO: | | |
| | | 38) | | |
| | A+ | SMPD | Potassium voltage-gated | Regulator against neuropsychiatric disorders |
| | C1 | ASLS | channel subfamily H | |
| | 5 | (SEQ | member 7 | |
| | | ID | Vomeronasal 2 receptor, | Production of pheromones present in neurons, microglia, |
| | | NO: | 54 | and astrocytes |
| | | 39) | | |
| | A+ | SASV | Atrophin-1 | Transcriptional corepressor, disruption of the gene has been |
| | C2 | HPLA | | linked to glial disruption and dentatorubral-pallidoluysian |
| | 17 | (SEQ | | atrophy and Parkinson's disease |
| | | ID | Engulfment and cell | Glia-mediated phagocytosis |
| | | NO: | motility protein 3 | |
| | | 41) | | |
| | A+ | SWTA | Neuropeptide S receptor | In dense networks of interwoven nerve fibers in branches |
| | C2 | YMSS | | and synapses, which are rich with glial filaments and are |
| | 21 | (SEQ | | active in plastic remodelling |
| | | ID | NPC intracellular | Regulates neuronal cholesterol and promoting mammalian |
| | | NO: | | |
| | | 41) | cholesterol transporter 1 | lifespan, and is essential in healthy neuroinflammation |
| | A+ | SHAH | Vomeronasal type-1 | Production of pheromones present in neurons, microglia, |
| | C2 | SLSV | receptor | and astrocytes |
| | 28 | (SEQ | | |
| | | ID | | |
| | | NO: | | |
| | | 44) | | |
| | A+ | SNPY | Neuron navigator 1 | Microtubule-associated protein is involved in neuronal |
| | C2 | SPSL | | migration |
| | 32 | (SEQ | Aggrecan core protein | Core ECM component that is expressed by developmental |
| | | ID | | astrocytes and has a major role in the control of glial cell |
| | | NO: | | maturation |
| | | 45) | LIM domain-binding 3 | In a subpopulation of protoplasmic adult glial cells during |
| | | | | CNS development |
| | A+ | SYLP | ATP-sensitive inward | Proliferation, differentiation and survival of neurons and |
| | C2 | DEFT | rectifier potassium | glial cells |
| | 6 | (SEQ | channel 10 | |
| | | ID | Interleukin-1 receptor- | Inflammatory mediators, and promote tumor growth, |
| | | NO: | associated kinase 1 | metastasis, immune suppression, and chemotherapy |
| | | 46) | | resistance |
| | A+ | STIQ | Stabilizer of axonemal | Development of ciliopathies and cancer |
| | C4− | FTPQ | microtubules 2 | |
| | 1* | (SEQ | | |
| | | ID | | |
| | | NO: | | |
| | | 47) | | |
| | A+ | STLS | Short transient receptor | Pathological events of neurological and psychiatric |
| | C4− | FTRM | potential channel 2 | disorders |
| | 2* | (SEQ | Adhesion G protein- | Astrocyte-neuron cell-cell ECM interaction, myelination, |
| | | ID | coupled receptor V1 | synaptic transmission, and plasticity |
| | | NO: | | |
| | | 48) | | |
| | A+ | SPLS | Thrombospondin-4 | Astrocyte modulation of cellular proliferation, migration, |
| | C4− | FQAT | | adhesion and attachment, synaptogenesis, and |
| | 9* | (SEQ | | inflammatory response to CNS injury and vascular |
| | | ID | | remodelling |
| | | NO: | ALS2 C-terminal-like | Astrocyte-mediated survival of spinal neurons in ALS |
| | | 50) | | model |
| | A+ | SNFP | CD180 antigen | Antigen expression in activated microglia and astrocytes in |
| | C4− | SLSV | | a cortical stab wound |
| | 16 | (SEQ | Netrin receptor DCC | Mediates axon attraction of neuronal growth cones in the |
| | | ID | | developing nervous system |
| | | NO: | | |
| | | 51) | | |
| | A+ | SVDD | Amyloid beta precursor- | Inflammatory mediator, and promote tumor growth, |
| | C5− | YFTR | like protein 1 | metastasis, immune suppression, and chemotherapy |
| | 1* | (SEQ | | resistance |
| | | ID | Protocadherin gamma a9 | Developing neuronal circuits and are mediators for glial |
| | | NO: | | morphology |
| | | 54) | | |
| | A+ | SIDY | Neuronal pentraxin | Uptake of synaptic material during synapse remodelling or |
| | C5− | MYAR | receptor | in mediating the synaptic clustering |
| | 3 | (SEQ | | |
| | | ID | | |
| | | NO: | | |
| | | 55) | | |
| Primed | AD | STTV | Leucine rich repeat | Component of postsynaptic density membrane and in |
| (Dex, | C4− | NTIA | containing 4C | glutamatergic synapses |
| D) | 11− | (SEQ | (Predicted), isoform | |
| | | ID | CRA_a | |
| | | NO: | Mucin apoprotein | Influences Alzheimer's disease pathogenesis and has been |
| | | 56) | | found in this proteome |
| | AD | STYH | Receptor tyrosine-protein | Cell surface receptor for neuregulins, establishing radial |
| | C5− | TNSS | kinase erbB-3 | glia and their transformation into astrocytes in cerebral |
| | 9− | (SEQ | | cortex |
| | | ID | | |
| | | NO: | | |
| | | 57) | | |
| | AD | SGSS | Misshapen-like kinase 1 | Required for normal synaptic density, dendrite complexity, |
| | C6− | FTMI | | as well as surface AMPA receptor expression in |
| | 5− | (SEQ | | hippocampal neurons |
| | | ID | | |
| | | NO: | | |
| | | 58) | | |

Postscripts * and − represent core clustered and un-clustered, but close, sequences, respectively, based on features described by the BLOSUM64 matrix.
Colours represent residues that are polar, non-polar, negatively charged, and positively charged.
Acronyms: adenosine triphosphate (ATP), central nervous system (CNS), amyotrophic lateral sclerosis (ALS), a-amino-3-hydroxy-5-methyl-4-isoxazolepropionic acid (AMPA), extracellular matrix (ECM), cluster differentiation (CD), Niemann-Pick disease(NPC), cyclic guanosine monophosphate (cGMP).

Glial cells exhibit a range of diverse functional phenotypes in vitro, which have been modeled through a sequence of activation and recovery treatments in vitro. In order to establish a set of phenotype-specific markers/ligands for diverse glial states, an enriched set of peptides that bind phenotypically unique glia were identified and validated using phage display bio-panning. Within this in vitro model the control group represents the ramified, or surveying, phenotype. This is exemplified by low secretion of pro-inflammatory cytokines and NO, minimal iNOS expression, and MHCII expression, and high BDNF and GDNF expression and morphologically smaller cell bodies with thin branching processes. In addition, microglial proliferation and phagocytosis are generally lower in surveying microglia.

Glia underwent a sequence of activating treatments and recoveries to model reactive or primed phenotypes. Activation was characterized by increased cytokine and NO release, high iNOS expression, and morphological transition into a flattened, round, amoeboid shape. Additionally, anti-inflammatory IL-10 levels increased with activation, hinting at potential recovery.

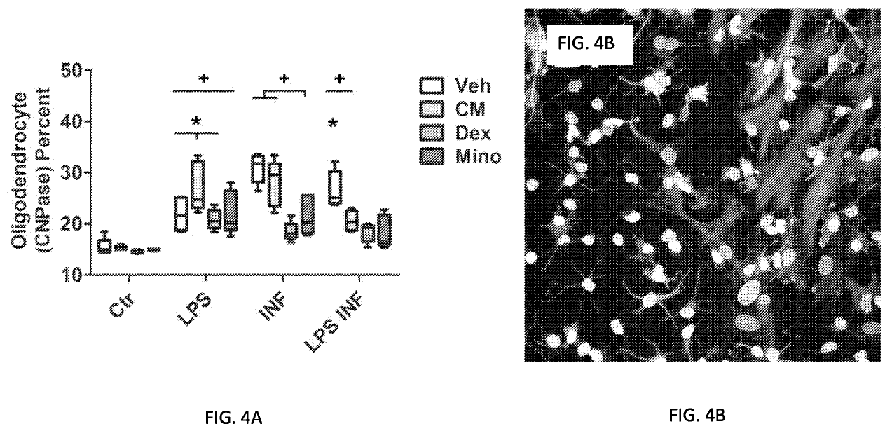

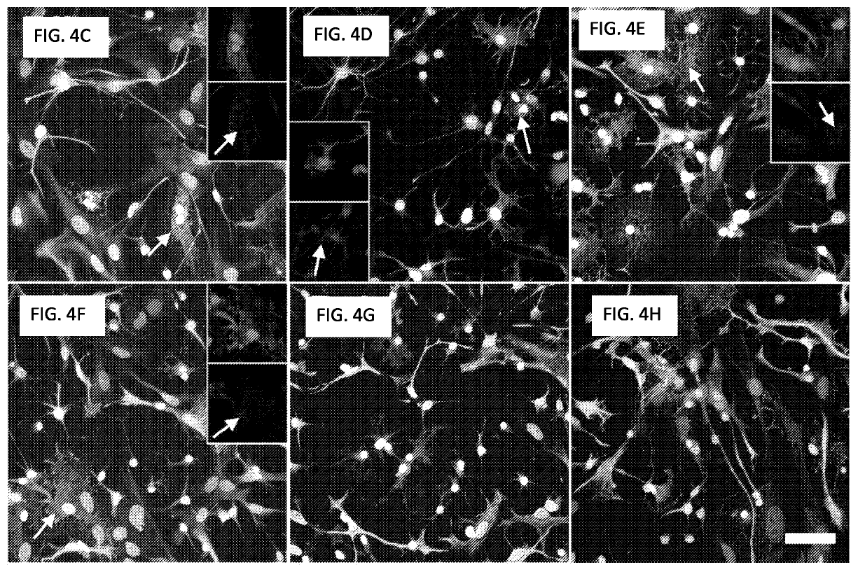

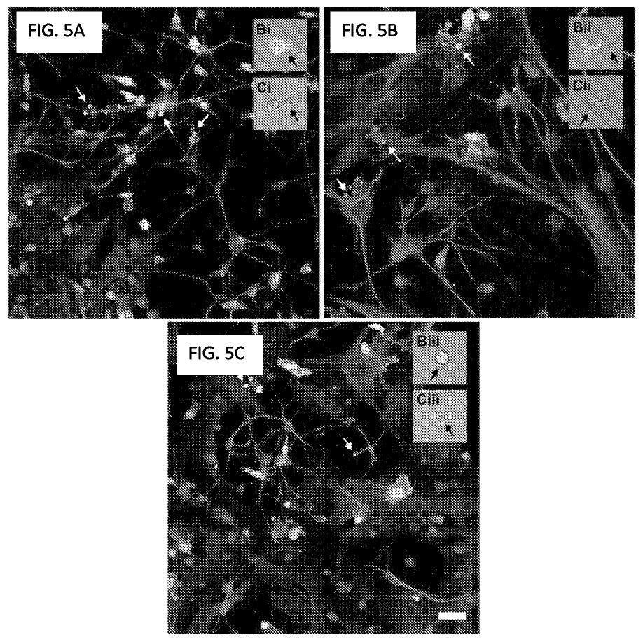

The nature of microglial priming was assessed after recovery treatments Mino, Dex, and CM, where LPS pre-treated cells were challenged with IFNγ. With the exception of nitrite secretion, Mino and CM were not able to reduce inflammatory secretions after recovery, nor were they able to attenuate this in interferon challenging. Mino was only able to contain this in isolated populations for NO and TNFα secretion. Due to these limitations, priming can't be presumed in these conditions. Dex recovery treatment generally suppressed cytokine release to comparable levels to control (FIGS. 2 and 3), however following Dex washout a subsequent IFN challenge failed to induce secretion of pro-inflammatory cytokines, NO, or IL10. This may be more consistent with evidence priming resulting in a dysfunctional inflammatory response rather than hyperfunction. All groups were able to attenuate myelin uptake with the LPS/INFγ challenge, however CM did not in other groups, i.e. LPS and INFγ and may have exacerbated this response with LPS (FIG. 4). Dex had a pronounced treatment effect across most of the dependent variables assessed. The reduction of IL10 secretion and neurotrophinsin particular, were reduced by only Dex, suggesting that any anti-inflammatory response in a reactive mixed cell microenvironment was lost due to this drug treatment (FIG. 2). In addition, isolated microglia were more readily primed than microglia in mixed glial populations. This may be due to support from other glial cells, notably astrocyte-microglial interaction. This was further supported by the observation that CM wasn't able to attenuate the inflammation comparably to the mixed population conditions. Given that LPS is considered a potent activator within the 6 hr timeline to activate microglia capable of likewise activating astrocytes, and that the Dex recovery was the most accurate at modelling priming, these treatments will likely give the most contrasting results in cell surface diversity and peptide binding. For these reasons, the surveying, activated, and primed phenotypes of microglia and astrocytes panned with SX7 phage, were modeled using LPS and Dex treatments in mixed populations. This is successfully noted in pre and post panning images of glia (FIG. 4).

Bioinformatic analysis (FIG. 5B-C) showed reactive microglia to have the lowest number of peptide clusters (2), but these peptides showed the highest amounts of normalized NCPM. This suggests that non-reactive microglia (surveying, primed) might have a higher diversity of surface expressed proteins capable of peptide binding, where reactive microglia may express a reduced complexity of more specialized proteins. This is consistent with the observation of a relatively low number of un-validated sequences (37/173) of reactive microglia but a high proportion of potent binders (14/24), which further reinforces the possibility that microglia exhibit a specialized reactive phenotype. Astrocytes tended to favour the opposite, where higher signal intensity with lower (3) clustering occurred in the surveying group, and increased clusters (5-6) were observed after activation. Astrocyte populations are changing with activation, but this data suggests they become heterogeneous in their expression of cell surface proteins. This reflected their non-uniform morphology and their ability to dampen inflammatory responses of microglia in the mixed cultures. Further, as reactive astrocytes were very high in clustering, un-validated binding events (192/296), and validated binding events (25/34), again supports that these cells have diverse binding capacity under high inflammatory stress. The identification of unique sequences in the primed state of both glial cell types suggests that this phenotype leads to significant cell surface adaptations. The vast majority of sequences (47/58) are within or immediately proximal to clustering from the BLOSUM62 analyses, suggesting that these peptides reflect physiological binding events and are not artefactual.

A brief BLAST survey of selected peptides suggested surveying microglial-specific peptides may have a diverse range of origins (Table 4). Some appeared to have roles in hormonal signaling such as thyroid hormone receptor, chorionic gonadotrophin transcription, or insulin receptors. As tissue collected was whole brain and likely included cells containing olfactory and retinal receptor analogues, several peptides have homology in olfactory receptor types and gamma-crystallin from retinal ganglion. Microglia have roles in a variety of hormonal regulation, some of which is linked to development and neuroinflammation, and they are present and specialized in olfactory and retinal tissues. A variety of synaptic support protein origins have also been identified, of which microglia are key players, Indicators of microglial inflammation have also been identified, including mediating newly assembled major histocompatibility complex (MHC) class I molecules, and T-cell differentiation or stimulation in dendritic cells, microglia, and neutrophils.

The surveying astrocytes-specific peptides suggest many possible origins, of which several support interactions with neurons, microglia, and endothelial cells (Table 5). Several peptides may originate from support proteins that attenuate brain injury through microglial accumulation along axons, promote angiogenesis and microglial-mediated brain vascularisation as a cellular chaperone for vessel fusion provide blood brain barrier endothelium support via astrocyte foot processes, drive neuritogenesis, assist in new neuronal cell division, and neurogenesis through microtubule polymerization. Astrocytes are a key player in these functions. Although less specialized for inflammation than microglia, astrocyte-specific peptides related to some neuroinflammatory pathways: neuroinflammatory acceleration due to astrocyte-neuron interaction with mast cells, inflammasome-positive astrocyte mediated recovery after severe brain injury, and lipid transport in neuroinflammation. One sequence may be associated with schizophrenia and excessive astrocyte-mediated synaptic pruning.

Reactive microglia-specific peptides (Table 4) originated from a variety of proteins associated with inflammatory insult. Several functions include T cell activation and innate immunity activation, endocytic and endosomal machinery, and cholesterol-lipid binding and uptake, which is indicative of myelin phagocytosis or lipid homeostasis. Some may be involved in glial proliferation, suggesting higher phosphorylation in inflammation, and be present in amyloid plaque-resident reactive microglia. One sequence identified relates to re-organizing of the ECM during neuroinflammation and gliomas. Several sequences have been linked to synaptic processing, neural cell adhesion, migration, memory plasticity, retrograde fast axonal transport, and glial-based gap junction neuroinflammatory interaction. Reactive microglia are a necessity in rescuing damaged tissues in many core neuro-inflammatory insults. It's possible that a substantial population of reactive microglia maintain this physiology, indicating that these cells have a robust capability to encourage axonal re-routing and the formation of new synaptic connections. The identified peptides might derive from parts of this machinery.

Peptides derived from reactive astrocytes (Table 5) were detected in several protein systems that are indicative of neural support and function in a variety ways: growth cone axonal attraction, imposing glial-mediated structures to neuronal circuits, and protoplasmic glia in the developing nervous system, direct neuronal activation, signal cell proliferation and apoptosis in synaptic plasticity, hippocampal development and polarization of neurons, neuronal migration, glial cell maturation, uptake of synaptic material during synapse remodeling or in mediating the synaptic clustering, proliferation, differentiation and survival of neurons and glial cells, crucial astrocyte-neuron cell-cell ECM myelination, synaptic transmission, and plasticity, plastic remodeling due to glial filament interwoven nerve fibers in dense network of branches and synapses, and astrocyte modulation of synaptogenesis, vascular remodeling, and inflammatory response to CNS injury. Several peptides originate from olfactory and vomeronasal receptors which may mediate sensory circuit processing and have a putatively function for the production of pheromones present in neurons, microglia, and astrocytes. Interestingly, several sequences originate from protein sources that are associated with neuroinflammation, immunity, and disease physiology. These may include neuronal-glial cholesterol regulation in healthy neuroinflammation, glial phagocytosis, antigen expression in activated microglia and astrocyte, and subdetectable retroviral activation of astrocytes linked to neurodegenerative disease. In addition, peptides from ion transporter protein loss may be linked to neurodegenerative and neuropsychiatric disorders, one specific to Parkinson's disease. Other peptides may be associated with amyotrophic lateral sclerosis astrocyte-mediated survival of spinal neurons and amyloid beta accumulation in Alzheimer's disease neuritic plaques. Some of the peptides are homologous to proteins that have a role in inflammatory mediation and promotion of tumor growth, metastasis, immune suppression, and chemotherapy resistance and the development of ciliopathies and cancer.

Peptides showing specificity for primed microglia (Table 4) show homology to a diverse range of proteins. Again, synaptic function is noted, focally inhibited in a NO synthase-dependent manner and are guided by cues to deflect axons from inappropriate regions. An ECM peptide was found exclusively associated with primed microglia, suggesting some residual microglial reactivity. Some peptides are linked to microglia and damaged mitotic machinery associated neurodegenerative and psychiatric diseases, and nociceptive hypersensitivity. One peptide is homologous to a protein with a direct role in glycosaminoglycan proteoglycans biosynthesis and may be crucial in glial scar interaction. Oligodendrocyte myelination is also implicated from homology to the 3F8 glycoprotein of the chondroitin sulfate proteoglycan of phosphacan, which is secreted by glia, binds to neurons and neural cell adhesion molecules. and is a potent inhibitor of cell adhesion and neurite outgrowth. A handful of peptides were specific for the primed astrocyte condition (Table 4). Similar to primed microglia, synaptic health might be a priority for these cells: maintenance of synaptic density, dendrite complexity in hippocampal neurons, and postsynaptic membrane integrity and in glutamatergic synapses. One peptide may facilitate radial glia transformation into astrocyte in the cerebral cortex, while another sequence is present in the Alzheimer's disease proteome and may influence pathogenesis

Support and synaptic maintenance were noted through all the phenotypes, however, this function was drastically reduced in the priming condition for both types of glia. These primed cells were noted to reduce their trophic support and anti-inflammatory function. Reactive microglia maintained a robust potential for neuronal support, while increasing their pro-inflammatory profile. On the other hand, reactive astrocytes appear to greatly expand their supportive repertoire, reflecting the diverse clustering noted from this phenotype. This is further reinforced as olfactory and vomeronasal function appeared to transfer from surveying microglia to reactive astrocytes.

Use of the discovered peptides may promote robust targeting of these cell types, which may lead to many biomaterial and clinical innovations.

### CONCLUSION

The phenotypes of surveying, reactive, and primed sub-types of microglia and their mixed neighbouring astrocytes were recreated. A custom 7 amino acid-presenting phage library was used to identify unique sequences that bind to these respective cell types. Surveying glia were untreated, reactive were induced with a lipopolysaccharide (LPS) treatment, recovery was modelled with a variety of anti-inflammatory treatments (minocycline, dexamethasone, and 24 hour conditioned media from a healthy mixed population), and priming was assessed by challenging the cells with interferon gamma (IFNγ). Microglial function was assessed by confocal microscopy with reactive marker expression (iNOS), secretion of cytokines (TNF-α, IL-1β, IL-10) and nitric oxide (NO). Dex proved to be the most physiologically relevant treatment and was used to for panning. Mixed cells were incubated with an SX7 M13 filamentous phage library. Populations of SX7-positive microglia and astrocytes were collected using FACS, amplified on E. coli culture, and sequenced via next-generation sequencing. Binding validation was done with synthesized peptides via in-cell westerns. A total of 58 unique peptides were discovered, and their potential functions were assessed using a BLAST search. Possible peptide functions ranged from a variety of supportive glial roles, including synapse support and pruning, inflammatory roles including cytokine and interleukin activation, and potential regulation in neurodegenerative and neuropsychiatric disorders. The results are an excellent proof of concept; demonstrating efficacy of the biopanning methods described herein for identifying peptides which may find use in downstream therapeutic methods for inflammation in the CNS.

TABLE 6
Summary of BLAST Identities for Surveying Microglia

| Code | Peptide | Entry | Description | Homology |
|---|---|---|---|---|
| M-C1-4- | SSLVL | P46152 | Transcription factor GATA-4 (Rattus norvegicus) | 100.00% |
| | ADS | F1M4Q3 | Hemicentin 1 (Rattus norvegicus) | 100.00% |
| | (SEQ ID | A0A096MJ40 | Hemicentin 1 (Rattus norvegicus) | 100.00% |
| | NO: 1) | F1M8B2 | Adenosine deaminase domain-containing 2 (Rattus | 100.00% |
| | | | norvegicus) | |
| | | P62516 | POU domain, class 3, transcription factor 4 (Rattus | 87.50% |
| | | | norvegicus) | |
| | | D3ZX88 | Olfactory receptor 14C36-like (Rattus norvegicus) | 100.00% |
| | | D4A3H7 | Olfactory receptor 34 (Rattus norvegicus) | 100.00% |
| | | Q5U2N3 | Membrane-associated phosphatidylinositol transfer protein | 85.70% |
| | | | 1 (Rattus norvegicus) | |
| | | B5DF37 | TAF6-like RNA polymerase II, p300/CBP-associated factor | 85.70% |
| | | | (PCAF)-associated factor (Predicted) (Rattus norvegicus) | |
| M-C2-13* | SSNSV | Q6P7A9 | Lysosomal alpha-glucosidase (Rattus norvegicus) | 100.00% |
| | VGI | D4ABR5 | Uncharacterized protein RGD1560175_predicted (Rattus | 100.00 |
| | (SEQ ID | | norvegicus) | |
| | NO: 2) | A0A1BOGWM3 | Uncharacterized protein (Rattus norvegicus) | 100.00% |
| | | M0R544 | Glucosidase, alpha, acid, isoform CRA_a (Rattus norvegicus) | 100.00% |
| | | D3ZPY5 | Olfactory receptor (Rattus norvegicus) | 100.00% |
| | | M0R8S5 | Olfactory receptor (Rattus norvegicus) | 100.00% |
| | | D3ZLT2 | Olfactory receptor (Rattus norvegicus) | 83.30% |
| M-C2-17* | SMSTY | P63059 | Thyroid hormone receptor alpha (Rattus norvegicus) | 75.00% |
| | VPS | G3V764 | Thyroid hormone receptor alpha (Rattus norvegicus) | 75.00% |
| | (SEQ ID | F1M649 | NLR family CARD domain-containing protein 4 (Rattus | 83.30% |
| | NO: 3) | | norvegicus) | |
| | | A0A096MJX4 | Fibrous sheath-interacting protein 2 (Rattus norvegicus) | 83.30% |
| | | D3ZN99 | Adhesion G protein-coupled receptor B2 (Rattus norvegicus) | 100.00% |
| | | A0A0G2K6N2 | Adhesion G protein-coupled receptor B2 (Rattus norvegicus) | 100.00% |
| | | Q9Z288 | Chorion-specific transcription factor GCMa (Rattus | 83.30% |
| | | | norvegicus) | |
| | | D4A224 | Protein RIC1 homolog (Rattus norvegicus) | 71.40% |
| | | A0A023IM54 | TAP binding protein (Rattus norvegicus) | 66.70% |
| | | Q99JC6 | TAP-binding protein (Rattus norvegicus) | 66.70% |
| M-C2-19* | STHTTS | Q4FZU8 | Rho family-interacting cell polarization regulator 1 (Rattus | 100.00% |
| | VL | | norvegicus) | |
| | (SEQ ID | A0A0G2JVR9 | Rho family-interacting cell polarization regulator 1 (Rattus | 100.00% |
| | NO: 4) | | norvegicus) | |
| | | D3ZQU7 | Glycoprotein Ib platelet subunit alpha (Rattus norvegicus) | 100.00% |
| M-C2-24 | SSSSLL | F8WG88 | Follistatin-related protein 1 (Rattus norvegicus) | 87.50% |
| | HY | Q7TP46 | Ab2-379 (Rattus norvegicus) | 87.50% |
| | (SEQ ID | P30191 | Gamma-aminobutyric acid receptor subunit alpha-6 (Rattus | 85.70% |
| | NO: 5) | | norvegicus) | |
| | | G3V6G3 | Gamma-aminobutyric acid (GABA-A) receptor, subunit alpha | 85.70% |
| | | | 6, isoform CRA_a (Rattus norvegicus) | |
| | | Q498R1 | Methionine synthase reductase (Rattus norvegicus) | 100.00% |
| | | Q923Y1 | Trace amine-associated receptor 7g (Rattus norvegicus) | 100.00% |
| | | A0A096MJW8 | Phosphoinositide phospholipase C (Rattus norvegicus) | 85.70% |
| | | D4A4H0 | Phosphoinositide phospholipase C (Rattus norvegicus) | 85.70% |
| | | F1LRH7 | Interleukin 12 receptor subunit beta 2 (Rattus norvegicus) | 100.00% |
| | | F7FLB2 | Pgm2 protein (Rattus norvegicus) | 100.00% |
| | | D4AEN3 | Olfactory receptor 869 (Rattus norvegicus) | 100.00% |
| | | A0A0G2K6N9 | COMM domain containing 4 (Predicted), isoform | 100.00% |
| | | | CRA_c (Rattus norvegicus) | |
| | | D4A2Q7 | COMM domain containing 4 (Predicted), isoform | 100.00% |
| | | | CRA_b (Rattus norvegicus) | |
| | | A0JPQ7 | Uncharacterized protein C19orf47 homolog (Rattus | 75.00% |
| | | | norvegicus) | |
| | | A0A0G2JVF4 | Similar to CG16812-PA (Rattus norvegicus) | 75.00% |
| | | Q9R1K2 | Teneurin-2 (Rattus norvegicus) | 100.00% |
| | | Q3ZBA0 | Tectonin beta-propeller repeat-containing protein 1 (Rattus | 100.00% |
| | | | norvegicus) | |
| | | Q673L6 | Telomerase reverse transcriptase (Rattus norvegicus) | 100.00% |
| | | Q4V7B1 | Protein CFAP20DC (Rattus norvegicus) | 100.00% |
| | | Q4V7C8 | Centrosomal protein of 55 kDa (Rattus norvegicus) | 100.00% |
| | | O35254 | Golgi reassembly-stacking protein 1 (Rattus norvegicus) | 100.00% |
| | | A0A096P6L6 | Teneurin-2 (Rattus norvegicus) | 100.00% |
| | | F1LRC7 | Similar to KIAA2026 protein (Rattus norvegicus) | 100.00% |
| | | A0A0G2JY22 | Disco-interacting protein 2 homolog B (Rattus norvegicus) | 100.00% |
| | | A0A0G2JTU1 | ERCC excision repair 6, chromatin-remodeling factor (Rattus | 100.00% |
| | | | norvegicus) | |
| | | F1MAL5 | Insulin receptor substrate 2 (Rattus norvegicus) | 100.00% |
| | | D3Z8G4 | ADAM metallopeptidase with thrombospondin type 1 motif, | 85.70% |
| | | | 18 (Rattus norvegicus) | |
| | | D3ZJ32 | Extended synaptotagmin 2 (Rattus norvegicus) | 100.00% |
| | | G3V8L0 | GATOR complex protein WDR24 (Rattus norvegicus) | 100.00% |
| | | F1M8H3 | Similar to RIKEN cDNA 4933406L09 (Rattus norvegicus) | 100.00% |
| | | A0A0G2JUF2 | Sorting nexin-30 (Rattus norvegicus) | 100.00% |
| | | D3ZH78 | G protein-regulated inducer of neurite outgrowth 2 (Rattus | 100.00% |
| | | | norvegicus) | |
| | | A0A0G2JWJ7 | Golgi reassembly-stacking protein 1 (Rattus norvegicus) | 100.00% |
| | | B1WBR1 | Golgi reassembly-stacking protein 1 (Rattus norvegicus) | 100.00% |
| | | D4A060 | Sorting nexin-30 (Rattus norvegicus) | 100.00% |
| | | F7EUU4 | COP9 signalosome subunit 5 (Rattus norvegicus) | 100.00% |
| | | Q4KM48 | Wdr24 protein (Rattus norvegicus) | 100.00% |
| | | Q4KM69 | COP9 (Constitutive photomorphogenic) homolog, subunit 5 | 100.00% |
| | | | (Arabidopsis thaliana) (Rattus norvegicus) | |
| | | A0A0G2JXD4 | Similar to RIKEN cDNA 4933406L09 (Rattus norvegicus) | 100.00% |
| | | Q5RJP3 | LOC361016 protein (Rattus norvegicus) | 100.00% |
| | | D4ACL4 | Homeo box C12 (Rattus norvegicus) | 100.00% |
| | | D4A1Q3 | Myb/SANT DNA-binding domain-containing 1 (Rattus | 100.00% |
| | | | norvegicus) | |
| | | Q6QI94 | LRRG00114 (Rattus norvegicus) | 100.00% |
| | | Q6TUI1 | LRRGT00063 (Rattus norvegicus) | 100.00% |
| | | O88948 | Insulin receptor substrate 2 (Rattus norvegicus) | 100.00% |
| | | A0JN16 | Gorasp1 protein (Rattus norvegicus) | 100.00% |
| | | Q68FU7 | Ubiquinone biosynthesis monooxygenase COQ6, | 83.30% |
| | | | mitochondrial (Rattus norvegicus) | |
| M-C2-44- | SHSYA | M0R8W9 | CD177 antigen-like (Rattus norvegicus) | 75.00% |
| | GVL | A0A0G2K8C9 | Gamma-crystallin A (Rattus norvegicus) | 100.00% |
| | (SEQ ID | M0R5G4 | Gamma-crystallin A (Rattus norvegicus) | 100.00% |
| | NO: 6) | | | |

TABLE 7
Summary of BLAST Identities for Activated Microglia

| Code | Peptide | Entry | Description | Homology |
|---|---|---|---|---|
| M+C1-1* | SSLTTL | Q9JKC9 | Synergin gamma (Rattus norvegicus) | 100.00% |
| | TA | P04157 | Receptor-type tyrosine-protein phosphatase C (Rattus | 100.00% |
| | (SEQ ID | | norvegicus) | |
| | NO: 7) | Q9Z1Y3 | Cadherin-2 (Rattus norvegicus) | 100.00% |
| | | Q6TUF6 | LRRGT00088 (Rattus norvegicus) | 100.00% |
| | | M0R8Z5 | HECT-type E3 ubiquitin transferase (Rattus norvegicus) | 100.00% |
| | | A0A0G2K0E7 | HECT-type E3 ubiquitin transferase (Rattus norvegicus) | 100.00% |
| | | G3V803 | Cadherin-2 (Rattus norvegicus) | 100.00% |
| | | A0A096MJZ7 | Uncharacterized protein (Rattus norvegicus) | 100.00% |
| | | A0A096MJG9 | Uncharacterized protein (Rattus norvegicus) | 100.00% |
| | | A0A0G2K0L4 | Uncharacterized protein (Rattus norvegicus) | 100.00% |
| | | G3V6Z2 | Potassium channel tetramerisation domain containing 6 | 100.00% |
| | | | (Predicted) (Rattus norvegicus) | |
| | | B0BNF4 | Kctd6 protein (Rattus norvegicus) | 100.00% |
| | | Q5R3Z4 | Uncharacterized protein (Rattus norvegicus) | 100.00% |
| | | P15257 | Hepatocyte nuclear factor 1-alpha (Rattus norvegicus) | 100.00% |
| | | Q8R4G8 | BTB/POZ domain-containing protein KCTD1 (Rattus | 85.70% |
| | | | norvegicus) | |
| | | A0A140TAI7 | BTB/POZ domain-containing protein KCTD1 (Rattus | 85.70% |
| | | | norvegicus) | |
| | | B5DF44 | Kctd15 protein (Rattus norvegicus) | 85.70% |
| | | O70594 | Solute carrier family 22 member 5 (Rattus norvegicus) | 85.70% |
| | | A0A0G2JVF2 | Solute carrier family 22 member 5 (Rattus norvegicus) | 85.70% |
| | | B2GUV4 | Slc22a5 protein (Rattus norvegicus) | 85.70% |
| M+C1-2* | SLASA | D4AB66 | Stonin-2 (Rattus norvegicus) | 85.70% |
| | VSN | D3ZZZ9 | Catenin (Cadherin associated protein), delta 1 (Predicted), | 100.00% |
| | (SEQ ID | | isoform CRA_a (Rattus norvegicus) | |
| | NO: 8) | Q04462 | Valine--tRNA ligase (Rattus norvegicus) | 100.00% |
| | | Q5M934 | Probable tRNA pseudouridine synthase 1 (Rattus norvegicus) | 85.70% |
| | | Q4FZR0 | Valyl-tRNA synthetase (Rattus norvegicus) | 100.00% |
| M+C1-3* | SSALT | M0RDY1 | Zinc finger protein 569 (Rattus norvegicus) | 72.70% |
| | VSG | P97661 | Zinc finger protein 5 (Rattus norvegicus) | 72.70% |
| | (SEQ ID | D3ZQH5 | Myotubularin-related protein 11 (Rattus norvegicus) | 85.70% |
| | NO: 9) | Q2WEA5 | Transient receptor potential cation channel subfamily M | 100.00% |
| | | | member 1 (Rattus norvegicus) | |
| | | F1M9X0 | Transient receptor potential cation channel subfamily M | 100.00% |
| | | | member 1 (Rattus norvegicus) | |
| | | D4ABR1 | Pleckstrin homology and RhoGEF domain-containing | 100.00% |
| | | | G6 (Rattus norvegicus) | |
| | | F1M4P7 | GATOR complex protein SEC13 (Rattus norvegicus) | 100.00% |
| | | D3ZJB8 | RBR-type E3 ubiquitin transferase (Rattus norvegicus) | 85.70% |
| M+C1-7- | SVAVE | M0R915 | NLR family, apoptosis inhibitory protein 2 (Rattus | 100.00% |
| | AHH | | norvegicus) | |
| | (SEQ ID | B1WC03 | AAR2 splicing factor homolog (Rattus norvegicus) | 100.00% |
| | NO: 10) | D3ZFR0 | FAST kinase domains 1 (Rattus norvegicus) | 83.30% |
| | | D4ADC4 | Kinesin family member 26B (Rattus norvegicus) | 62.50% |
| | | Q3KRC5 | tRNA-dihydrouridine(47) synthase [NAD(P)(+)]-like (Rattus | 83.30% |
| | | | norvegicus) | |
| | | D3ZDR7 | Thymocyte selection-associated family member 2 (Rattus | 83.30% |
| | | | norvegicus) | |
| | | M0RAY0 | tRNA-dihydrouridine(47) synthase [NAD(P)(+)] (Rattus | 83.30% |
| | | | norvegicus) | |
| M+C1-8- | SHIGTV | A0A0G2K5S0 | Cation channel sperm-associated auxiliary subunit | 83.30% |
| | TG | | gamma (Rattus norvegicus) | |
| | (SEQ ID | G3V9G0 | Protocadherin beta 21 (Rattus norvegicus) | 83.30% |
| | NO: 11) | B2RZC3 | RGD1310942 protein (Rattus norvegicus) | 83.30% |
| M+C1-12- | SVMTI | Q99P39 | Cysteine desulfurase, mitochondrial (Rattus norvegicus) | 85.70% |
| | NNT | Q3MHT2 | Cysteine desulfurase, mitochondrial (Rattus norvegicus) | 85.70% |
| | (SEQ ID | Q5FWU2 | Cysteine desulfurase, mitochondrial (Rattus norvegicus) | 85.70% |
| | NO: 12) | D4A240 | BRD4-interacting chromatin-remodeling complex-associated | 85.70% |
| | | | protcin-like (Rattus norvegicus) | |
| | | A0A0G2K0D5 | Oxysterol-binding protein (Rattus norvegicus) | 83.30% |
| | | D4A0G0 | Oxysterol-binding protein (Rattus norvegicus) | 83.30% |
| | | F1M4P7 | GATOR complex protein SEC13 (Rattus norvegicus) | 83.30% |
| M+C1-15- | SAQTL | F1LU96 | NCK-associated protein 5 (Rattus norvegicus) | 100.00% |
| | DRS | M0R6D9 | RING-type E3 ubiquitin transferase (Rattus norvegicus) | 85.70% |
| | (SEQ ID | P22199 | Mineralocorticoid receptor (Rattus norvegicus) | 83.30% |
| | NO: 13) | B4F7D6 | Serine/threonine-protein kinase 11-interacting protein (Rattus | 83.30% |
| | | | norvegicus) | |
| | | A0A0G2K9H2 | Serine/threonine-protein kinase 11-interacting protein (Rattus | 83.30% |
| | | | norvegicus) | |
| | | F1M6V2 | Mineralocorticoid receptor (Rattus norvegicus) | 83.30% |
| | | Q63722 | Protein jagged-1 (Rattus norvegicus) | 71.40% |
| | | G3V710 | Delta-like protein (Rattus norvegicus) | 71.40% |
| M+C1-16- | SLIEW | D3ZI66 | Ring finger protein 149 (Rattus norvegicus) | 100.00% |
| | YSA | A0A0G2K698 | Lamin tail domain-containing 1 (Rattus norvegicus) | 100.00% |
| | (SEQ ID | D4ABG2 | Lamin tail domain-containing 1 (Rattus norvegicus) | 100.00% |
| | NO: 14) | P08050 | Gap junction alpha-1 protein (Rattus norvegicus) | 80.00% |
| | | A0A654ICE6 | Connexin-43 (Rattus norvegicus) | 80.00% |
| | | P10688 | 1-phosphatidylinositol 4,5-bisphosphate phosphodiesterase | 83.30% |
| | | | delta-1 (Rattus norvegicus) | |
| | | D3ZFT1 | Replication factor C subunit 1 (Rattus norvegicus) | 83.30% |
| | | Q9Z2R7 | Replication factor C subunit 1 (Rattus norvegicus) | 83.30% |
| | | D4A7G5 | Glucosidase, alpha; neutral C (Rattus norvegicus) | 100.00% |
| | | A6Y857 | Phosphoinositide phospholipase C (Rattus norvegicus) | 83.30% |
| | | G3V9D1 | Phosphoinositide phospholipase C (Rattus norvegicus) | 83.30% |
| | | M0R829 | Uncharacterized protein (Rattus norvegicus) | 83.30% |
| | | A0A0G2K2W3 | Uncharacterized protein (Rattus norvegicus) | 83.30% |
| | | A0A0G2JVK8 | Replication factor C subunit 1 (Rattus norvegicus) | 83.30% |
| | | O88461 | VIP-receptor-gene repressor protein (Rattus norvegicus) | 83.30% |
| | | D3ZD71 | EF-hand domain-containing 1 (Rattus norvegicus) | 80.00% |
| | | D3ZV56 | Ring finger protein 150 (Rattus norvegicus) | 80.00% |
| M+C1-20- | SYMNN | P09811 | Glycogen phosphorylase, liver form (Rattus norvegicus) | 83.30% |
| | SVF | Q5PQT2 | Myotubularin-related protein 3 (Rattus norvegicus) | 100.00% |
| | (SEQ ID | Q9R1Q2 | Cyclin-L1 (Rattus norvegicus) | 54.50% |
| | NO: 15) | Q510H5 | Cyclin-L2 (Rattus norvegicus) | 54.50% |
| | | Q9R1A0 | Cyclin-H (Rattus norvegicus) | 83.30% |
| | | A0A0G2K8C7 | Phosphatidylinositol-3-phosphate phosphatase (Rattus | 100.00% |
| | | | norvegicus) | |
| | | F1M8D3 | C-Maf-inducing protein (Rattus norvegicus) | 83.30% |
| | | A0A0G2JYB9 | C-Maf-inducing protein (Rattus norvegicus) | 83.30% |
| | | A0A0H2UHQ5 | Cyclin-L2 (Rattus norvegicus) | 54.50% |
| | | B1WBN1 | Cyclin-L1 (Rattus norvegicus) | 54.50% |
| | | Q63170 | Dynein axonemal heavy chain 7 (Rattus norvegicus) | 83.30% |
| | | A0A0G2JYD0 | Dynein heavy chain 7, axonemal (Rattus norvegicus) | 83.30% |
| | | A0A1W2Q624 | Dynein heavy chain 7, axonemal (Rattus norvegicus) | 83.30% |
| | | M0R3Q7 | Patched domain-containing 3 (Rattus norvegicus) | 100.00% |
| | | F1MA76 | Glutamate-rich 3 (Rattus norvegicus) | 80.00% |
| M+C1-22- | SDTPTS | Q5D006 | Ubiquitin carboxyl-terminal hydrolase 11 (Rattus norvegicus) | 85.70% |
| | PG | D4A7K5 | Phospholipid-transporting ATPase (Rattus norvegicus) | 100.00% |
| | (SEQ ID | A0A0G2K8V1 | Phospholipid-transporting ATPase (Rattus norvegicus) | 100.00% |
| | NO: 16) | D3ZFC5 | Phospholipid-transporting ATPase (Rattus norvegicus) | 100.00% |
| | | A0A0G2K3P7 | Phospholipid-transporting ATPase (Rattus norvegicus) | 100.00% |
| | | Q6IE71 | Ubiquitin specific protease 11 (Rattus norvegicus) | 85.70% |
| | | D4A6B3 | SLAM family member 6 (Rattus norvegicus) | 87.50% |
| | | Q9ESI7 | Neuronal migration protein doublecortin (Rattus norvegicus) | 100.00% |
| | | A0A0G2JYE0 | Ataxin 2-like (Rattus norvegicus) | 100.00% |
| | | B3DMA1 | Ataxin 2-like (Rattus norvegicus) | 100.00% |
| | | A0A0G2K0M8 | Neural cell adhesion molecule 1 (Rattus norvegicus) | 100.00% |
| | | A0A0G2JZZ7 | Active breakpoint cluster region-related protein (Rattus | 100.00% |
| | | | norvegicus) | |
| | | D4ACD8 | Homeo box D10 (Rattus norvegicus) | 100.00% |
| | | G3V997 | Neuronal migration protein doublecortin (Rattus norvegicus) | 100.00% |
| | | Q64535 | Copper-transporting ATPase 2 (Rattus norvegicus) | 75.00% |
| | | A1L1J6 | Zinc finger protein 652 (Rattus norvegicus) | 83.30% |
| | | A0A0G2JWJ5 | P-type Cu(+) transporter (Rattus norvegicus) | 75.00% |
| | | Q9QUG4 | P-type Cu(+) transporter (Rattus norvegicus) | 75.00% |
| | | Q9R0T2 | P-type Cu(+) transporter (Rattus norvegicus) | 75.00% |
| | | Q6AY32 | CEP295 N-terminal-like (Rattus norvegicus) | 83.30% |
| | | D3ZG83 | Mitogen-activated protein kinase kinasekinase 10 (Rattus | 85.70% |
| | | | norvegicus) | |
| | | A0A0G2JVD6 | [Histone H3]-lysine(4) N-trimethyltransferase (Rattus | 83.30% |
| | | | norvegicus) | |
| M+C1-23- | SDPSQF | F1M9C5 | Runt-related transcription factor (Rattus norvegicus) | 85.70% |
| | PL (SEQ | D3ZA12 | Chromodomain-helicase-DNA-binding protein 6 (Rattus | 83.30% |
| | ID NO: | | norvegicus) | |
| | 17) | Q9JIX5 | Chromodomain-helicase-DNA-binding protein 8 (Rattus | 83.30% |
| | | | norvegicus) | |
| | | A0A0G2K1L7 | DNA helicase (Rattus norvegicus) | 83.30% |
| | | D3ZU90 | Fer-1-like family member 5 (Rattus norvegicus) | 71.40% |
| | | D3Z8E0 | Ribosomal protein S6 kinase (Rattus norvegicus) | 85.70% |
| | | F1M7N7 | Ribosomal protein S6 kinase (Rattus norvegicus) | 85.70% |
| | | Q99NH5 | GATA-binding factor 3 (Rattus norvegicus) | 71.40% |
| | | D3ZUI1 | Methylthioribulose-1-phosphate dehydratase (Rattus | 83.30% |
| | | | norvegicus) | |
| | | B4F7E8 | Protein Niban 2 (Rattus norvegicus) | 83.30% |
| | | D4A116 | Phospholipase A2 (Rattus norvegicus) | 83.30% |
| | | A0A0G2JZ44 | Phospholipase A2 (Rattus norvegicus) | 83.30% |
| | | D4A5C9 | Sterile alpha motif domain-containing 7 (Rattus norvegicus) | 100.00% |
| | | D4A7V8 | Uncharacterized protein RGD1562464_predicted (Rattus | 83.30% |
| | | | norvegicus) | |
| M+C1-27- | SHLSY | B1WBR8 | F-box and leucine-rich repeat protein 4 (Rattus norvegicus) | 83.30% |
| | TMW | Q9ESE8 | Inhibitor of apoptosis protein 2 (Rattus norvegicus) | 83.30% |
| | (SEQ ID | Q9QZC6 | Inhibitor of apoptosis protein 2 (Rattus norvegicus) | 83.30% |
| | NO: 18) | Q6P6S1 | Baculoviral IAP repeat-containing 2 (Rattus norvegicus) | 83.30% |
| | | O88642 | Inhibitor of apoptosis protein (Rattus norvegicus) | 83.30% |
| | | Q08469 | Sodium-dependent neutral amino acid transporter | 83.30% |
| | | | B(0)AT2 (Rattus norvegicus) | |
| | | O88664 | Serine/threonine-protein kinase TAO1 (Rattus norvegicus) | 83.30% |
| | | F1LV37 | Trinucleotide repeat-containing adaptor 6B (Rattus | 71.40% |
| | | | norvegicus) | |
| | | A0A0G2K6R0 | Trinucleotide repeat-containing adaptor 6B (Rattus | 71.40% |
| | | | norvegicus) | |
| | | F1M9G6 | Serine/threonine-protein kinase TAO1 (Rattus norvegicus) | 83.30% |
| | | Q6P7B1 | Tnrc6b protein (Rattus norvegicus) | 71.40% |
| | | Q4KLZ0 | Vanin 1 (Rattus norvegicus) | 83.30% |
| M+C1-32- | SGNED | D3ZZ42 | Matrix metallopeptidase 21 (Rattus norvegicus) | 71.40% |
| | RYP | O88277 | Protocadherin Fat 2 (Rattus norvegicus) | 83.30% |
| | (SEQ ID | P48442 | Extracellular calcium-sensing receptor (Rattus norvegicus) | 62.50% |
| | NO: 19) | F1LPM1 | Protocadherin Fat 2 (Rattus norvegicus) | 83.30% |
| | | Q80ZA8 | Calcium sensing receptor (Rattus norvegicus) | 62.50% |
| M+C1-33- | SHDDIP | D3ZP45 | Polymerase (RNA) III (DNA directed) polypeptide E | 100.00% |
| | HL | | (Predicted) (Rattus norvegicus) | |
| | (SEQ ID | P97691 | Forkhead box protein M1 (Rattus norvegicus) | 83.30% |
| | NO: 20) | D3ZLE1 | Forkhead box protein M1 (Rattus norvegicus) | 83.30% |
| | | F1LN13 | Forkhead box protein M1 (Rattus norvegicus) | 83.30% |
| | | Q9QZC4 | Choline-phosphate cytidylyltransferase B (Rattus norvegicus) | 100.00% |
| | | P19836 | Choline-phosphate cytidylyltransferase A (Rattus norvegicus) | 100.00% |
| | | M0R4J5 | Interferon-induced protein 44-like (Rattus norvegicus) | 100.00% |
| | | G3V7M5 | Choline-phosphate cytidylyltransferase B (Rattus norvegicus) | 100.00% |
| | | A0A096MK76 | Choline-phosphate cytidylyltransferase B (Rattus norvegicus) | 100.00% |
| | | A0A0G2KAX1 | EEF1A lysine methyltransferase 1 (Rattus norvegicus) | 83.30% |
| | | B5DEG5 | EEF1A lysine methyltransferase 1 (Rattus norvegicus) | 83.30% |
| | | A0A096MK28 | EEF1A lysine methyltransferase 1 (Rattus norvegicus) | 83.30% |

TABLE 8
Summary of BLAST Identities for Primed Microglia

| Code | Peptide | Entry | Description | Homology |
|---|---|---|---|---|
| MDC1-6- | SVDSN | A0A0G2K1X3 | WD repeat domain 60 (Rattus norvegicus) | 100.00% |
| | NEH | D3ZYV1 | WD repeat domain 60 (Rattus norvegicus) | 100.00% |
| | (SEQ ID | Q8R515 | Zinc fingers and homeoboxes protein 1 (Rattus norvegicus) | 62.50% |
| | NO: 21) | D3ZU13 | Eukaryotic translation initiation factor 4 gamma, 1 (Rattus | 71.40% |
| | | | norvegicus) | |
| | | D4AD15 | Eukaryotic translation initiation factor 4 gamma, 1 (Rattus | 71.40% |
| | | | norvegicus) | |
| | | A0A0G2JWH3 | Condensin complex subunit 1 (Rattus norvegicus) | 71.40% |
| | | G3V6S9 | RCG60240, isoform CRA_a (Rattus norvegicus) | 62.50% |
| | | M0RD03 | Eukaryotic translation initiation factor 4 gamma 1-like (Rattus | 71.40% |
| | | | norvegicus) | |
| | | Q9EPI1 | Xylosyltransferase 1 (Rattus norvegicus) | 100.00% |
| | | E3W9F8 | Laminin, alpha 1 (Rattus norvegicus) | 83.30% |
| | | D4A409 | Laminin subunit alpha 1 (Rattus norvegicus) | 83.30% |
| | | A0A0G2KB96 | STIL, centriolar assembly protein (Rattus norvegicus) | 85.70% |
| | | A0A0G2K6K1 | Peptide O-xylosyltransferase 1 (Rattus norvegicus) | 100.00% |
| | | D4ACX8 | Protocadherin-16 (Rattus norvegicus) | 83.30% |
| | | D4ABD7 | Thyroid hormone receptor interactor 11 (Rattus norvegicus) | 83.30% |
| | | F1M7Z6 | Uncharacterized protein (Rattus norvegicus) | 83.30% |
| MDC1-17- | SFGFM | D3ZZK0 | EF-hand calcium-binding domain 7 (Rattus norvegicus) | 83.30% |
| | NLH | D3Z8E2 | Sema domain, immunoglobulin domain (Ig), short basic | 71.40% |
| | (SEQ ID | | domain, secreted, (Semaphorin) 3E (Predicted) (Rattus | |
| | NO: 22) | | norvegicus) | |
| | | A0A0G2K377 | Semaphorin 3E (Rattus norvegicus) | 71.40% |
| | | P07825 | Synaptophysin (Rattus norvegicus) | 83.30% |
| | | G3V9C4 | ST6 (Alpha-N-acetyl-neuraminyl-2,3-beta-galactosyl-1, 3)-N- | 83.30% |
| | | | acetylgalactosaminide alpha-2,6-sialyltransferase 1 (Rattus | |
| | | | norvegicus) | |
| | | Q6ZYN7 | Alpha-2,6-sialyltransferase ST6GalNAc I (Rattus norvegicus) | 83.30% |
| | | A0A0G2KAD8 | Synaptophysin (Rattus norvegicus) | 83.30% |
| | | Q9WVR3 | Phosphatidylinositol 3,4,5-trisphosphate 5-phosphatase | 83.30% |
| | | | 2 (Rattus norvegicus) | |
| | | A6P320 | E3 ubiquitin-protein ligase MARCHF11 (Rattus norvegicus) | 66.70% |
| | | V9GZ88 | Phosphatidylinositol-3,4,5-trisphosphate 5- | 83.30% |
| | | | phosphatase (Rattus norvegicus) | |
| | | A0A1B0GWM0 | Phosphatidylinositol-3,4,5-trisphosphate 5- | 83.30% |
| | | | phosphatase (Rattus norvegicus) | |
| | | D4A6H3 | STEAP2 metalloreductase (Rattus norvegicus) | 71.40% |
| | | FILSY5 | RING-type E3 ubiquitin transferase (Rattus norvegicus) | 66.70% |
| MDC4-4- | STHVQ | Q505J3 | WSC domain-containing protein 1 (Rattus norvegicus) | 62.50% |
| | NWL | A0A0G2KAQ9 | Telomere repeat-binding bouquet formation protein 1 (Rattus | 100.00% |
| | (SEQ ID | | norvegicus) | |
| | NO: 23) | A0A0G2KAH8 | Telomere repeat-binding bouquet formation protein 1 (Rattus | 100.00% |
| | | | norvegicus) | |
| | | E9PU24 | Dynein, axonemal, heavy chain 11 (Rattus norvegicus) | 66.70% |
| | | F1M8M0 | RAB44, member RAS oncogene family (Rattus norvegicus) | 71.40% |
| MDC1-6- | SNIMH | Q6IE24 | Inactive ubiquitin carboxyl-terminal hydrolase 54 (Rattus | 83.30% |
| | SLN | | norvegicus) | |
| | (SEQ ID | A0A0G2JTN3 | Inactive ubiquitin carboxyl-terminal hydrolase 54 (Rattus | 83.30% |
| | NO: 24) | | norvegicus) | |
| | | D4AAM5 | Leucine-rich repeat-containing protein PRAME-like (Rattus | 83.30% |
| | | | norvegicus) | |
| | | D5MTH0 | E3 ubiquitin-protein ligase (Rattus norvegicus) | 71.40% |
| | | D4A9U6 | E3 ubiquitin-protein ligase (Rattus norvegicus) | 71.40% |
| | | A0A0G2K1U4 | E3 ubiquitin-protein ligase (Rattus norvegicus) | 71.40% |
| | | D4AC97 | EMSY transcriptional repressor, BRCA2-interacting (Rattus | 83.30% |
| | | | norvegicus) | |
| | | Q32PY1 | E3 ubiquitin-protein ligase (Rattus norvegicus) | 71.40% |
| | | Q66H04 | F-box only protein 43 (Rattus norvegicus) | 85.70% |
| | | D4A634 | RAN-binding protein 6 (Rattus norvegicus) | 71.40% |
| | | Q62656 | Receptor-type tyrosine-protein phosphatase zeta (Rattus | 83.30% |
| | | | norvegicus) | |
| | | F1LMY3 | Protein-tyrosine-phosphatase (Rattus norvegicus) | 83.30% |
| | | O08662 | Phosphatidylinositol 4-kinase alpha (Rattus norvegicus) | 75.00% |
| | | P11661 | NADH-ubiquinone oxidoreductase chain 5 (Rattus | 83.30% |
| | | | norvegicus) | |
| | | A0A140TAJ5 | Phosphatidylinositol 4-kinase alpha (Rattus norvegicus) | 75.00% |
| | | A0A096XKT9 | NADH-ubiquinone oxidoreductase chain 5 (Rattus | 83.30% |
| | | | norvegicus) | |
| | | Q06QK5 | NADH-ubiquinone oxidoreductase chain 5 (Rattus | 83.30% |
| | | | norvegicus) | |
| | | Q06QA1 | NADH-ubiquinone oxidoreductase chain 5 (Rattus | 83.30% |
| | | | norvegicus) | |
| | | Q06QG6 | NADH-ubiquinone oxidoreductase chain 5 (Rattus | 83.30% |
| | | | norvegicus) | |
| | | Q8SEZ0 | NADH-ubiquinone oxidoreductase chain 5 (Rattus | 83.30% |
| | | | norvegicus) | |

TABLE 9
Summary of BLAST Identities for Surveying Astrocytes

| Code | Peptide | Entry | Description | Homology |
|---|---|---|---|---|
| A-C1-3 | SNVID | O08722 | Netrin receptor UNC5B (Rattus norvegicus) | 83.30% |
| | PAN | F1LNE2 | Netrin receptor UNC5 (Rattus norvegicus) | 83.30% |
| | (SEQ ID | B5DF47 | KRIT1, ankyrin repeat containing (Rattus norvegicus) | 83.30% |
| | NO: 25) | G3V8Z6 | KRIT1, ankyrin repeat-containing (Rattus norvegicus) | 83.30% |
| A-C2-1* | SGNST | F1M6T0 | Fibronectin type III domain-containing 3C1 (Rattus | 100.00% |
| | SNA | | norvegicus) | |
| | (SEQ ID | A0A0U1RRN5 | Fibronectin type III domain-containing 3C1 (Rattus | 100.00% |
| | NO: 26) | | norvegicus) | |
| | | A0A0G2K0A1 | Fibronectin type III domain-containing 3C1 (Rattus | 100.00% |
| | | | norvegicus) | |
| | | Q9R1R4 | Tudor domain-containing protcin 7 (Rattus norvegicus) | 100.00% |
| A-C2-2* | SSTVA | C6K2K4 | Neuropilin and tolloid-like protein 2 (Rattus norvegicus) | 100.00% |
| | NDG | A0A096MJX4 | Fibrous sheath-interacting protein 2 (Rattus norvegicus) | 85.70% |
| | (SEQ ID | D4AAD6 | Neuropilin and tolloid-like 1 (Rattus norvegicus) | 100.00% |
| | NO: 27) | F1MA16 | Neuropilin and tolloid-like protein 2 (Rattus norvegicus) | 100.00% |
| A-C2-6 | SPNTN | A0A140UHX3 | Condensin-2 complex subunit D3 (Rattus norvegicus) | 85.70% |
| | TLA | Q3T1H0 | Condensin-2 complex subunit D3 (Rattus norvegicus) | 85.70% |
| | (SEQ ID | P97592 | Mast cell protease 4 (Rattus norvegicus) | 83.30% |
| | NO: 28) | Q63623 | SR-related and CTD-associated factor 8 (Rattus norvegicus) | 83.30% |
| | | D4A2D3 | RCR-type E3 ubiquitin transferase (Rattus norvegicus) | 85.70% |
| | | A0A1W2Q616 | MYC-binding protein 2 (Rattus norvegicus) | 85.70% |
| | | F1LPT8 | SR-related and CTD-associated factor 8 (Rattus norvegicus) | 83.30% |
| | | A0A0G2K5M7 | SR-related and CTD-associated factor 8 (Rattus norvegicus) | 83.30% |
| A-C2-12 | SVFNV | Q5XHY0 | RNA helicase (Rattus norvegicus) | 83.30% |
| | NNT | Q5U300 | Ubiquitin-like modifier-activating enzyme 1 (Rattus | 83.30% |
| | (SEQ ID | | norvegicus) | |
| | NO: 29) | Q62927 | cGMP-gated cation channel alpha-1 (Rattus norvegicus) | 100.00% |
| | | F1LQN0 | Cyclic nucleotide-gated cation channel 1 (Rattus norvegicus) | 100.00% |
| | | D4A6L0 | Probable G-protein coupled receptor 158 (Rattus norvegicus) | 83.30% |
| | | A0A0A0MY13 | Probable G-protein-coupled receptor 158 (Rattus norvegicus) | 83.30% |
| | | A0A0S1LIA8 | Cytoplasmic activation/proliferation-associated protein | 100.00% |
| | | | 2 (Rattus norvegicus) | |
| A-C3-5- | SAYLD | D4A7J3 | ATP-binding cassette subfamily A member 12 (Rattus | 85.70% |
| | SLN | | norvegicus) | |
| | (SEQ ID | Q6AYG3 | Exopolyphosphatase PRUNE1 (Rattus norvegicus) | 85.70% |
| | NO: 30) | F1LS35 | Hermansky-Pudlak syndrome 5 protein homolog (Rattus | 100.00% |
| | | | norvegicus) | |
| | | A0A0G2JWL2 | Hermansky-Pudlak syndrome 5 protein homolog (Rattus | 100.00% |
| | | | norvegicus) | |
| | | Q7TMC3 | Hermansky-Pudlak syndrome 5 protein homolog (Rattus | 100.00% |
| | | | norvegicus) | |
| | | D3ZD23 | ATP-binding cassette subfamily E member 1 (Rattus | 100.00% |
| | | | norvegicus) | |
| | | F1M8F6 | Myosin-8 (Rattus norvegicus) | 85.70% |
| | | Q9ESR9 | ATP-binding cassette sub-family A member 2 (Rattus | 83.30% |
| | | | norvegicus) | |
| | | Q924V4 | Solute carrier family 15 member 3 (Rattus norvegicus) | 83.30% |
| | | A0A0G2K6F2 | CUB and Sushi multiple domains 2 (Rattus norvegicus) | 83.30% |
| | | G3V7X4 | ATP-binding cassette sub-family A member 2 (Rattus | 83.30% |
| | | | norvegicus) | |
| | | D3ZDM5 | NACHT, leucine rich repeat and PYD containing 5 | 83.30% |
| | | | (Predicted) (Rattus norvegicus) | |
| | | Q29RW1 | Myosin-4 (Rattus norvegicus) | 85.70% |
| | | F1LMU0 | Myosin heavy chain 4 (Rattus norvegicus) | 85.70% |
| | | A0A0G2K484 | Myosin heavy chain 2 (Rattus norvegicus) | 85.70% |
| | | P39069 | Adenylate kinase isoenzyme 1 (Rattus norvegicus) | 100.00% |
| | | A0A0G2K7Q6 | Nucleoside-diphosphate kinase (Rattus norvegicus) | 100.00% |
| | | M0RC66 | Nucleoside-diphosphate kinase (Rattus norvegicus) | 100.00% |
| | | D4A3G9 | Patatin-like phospholipase domain-containing 1 (Rattus | 100.00% |
| | | | norvegicus) | |

TABLE 10
Summary of BLAST Identities for Activated Astrocytes

| | | | | Ho- |
|---|---|---|---|---|
| Code | Peptide | Entry | Description | mology |
| A + | SVSTTGVR | F1LZ43 | Disco-interacting protein 2 homolog A (Rattus norvegicus) | 85.70% |
| C1-1* | (SEQ ID | A0A0G2KB74 | Disco-interacting protein 2 homolog A (Rattus norvegicus) | 85.70% |
| | NO: 31) | D3ZZ97 | Receptor tyrosine kinase-like orphan receptor 1 (Rattus | 100.00% |
| | | | norvegicus) | |
| | | D3ZZB0 | Disco-interacting protein 2 homolog C (Rattus norvegicus) | 100.00% |
| | | A0A0G2JY22 | Disco-interacting protein 2 homolog B (Rattus norvegicus) | 100.00% |
| | | D3ZA02 | Family with sequence similarity 13, member A (Rattus | 100.00% |
| | | | norvegicus) | |
| | | D3ZGJ3 | Zinc finger and BTB domain containing 37 | 100.00% |
| | | | (Predicted) (Rattus norvegicus) | |
| | | Q5M847 | Fam13a1 protein (Rattus norvegicus) | 100.00% |
| A + | SFSNIAIR | D4AEJ5 | Similar to BH3-only member B protein (Predicted) (Rattus | 83.30% |
| C1-2* | (SEQ ID | | norvegicus) | |
| | NO: 32) | P08154 | Early growth response protein 1 (Rattus norvegicus) | 83.30% |
| | | A0A0H2UHR3 | Early growth response protein (Rattus norvegicus) | 83.30% |
| | | D3ZFM4 | Ditrans,polycis-polyprenyl diphosphate synthase ((2E,6E)- | 83.30% |
| | | | farnesyldiphosphate specific) (Rattus norvegicus) | |
| | | P97705 | Krox-24 (Rattus norvegicus) | 83.30% |
| A + | SLSAVDVS | O55173 | 3-phosphoinositide-dependent protein kinase 1 (Rattus | 87.50% |
| C1-5 | (SEQ ID | | norvegicus) | |
| | NO: 33) | A0A0G2K9R7 | Uncharacterized protein (Rattus norvegicus) | 85.70% |
| | | Q62623 | Cell division cycle protein 20 homolog (Rattus norvegicus) | 100.00% |
| | | D3ZVG1 | GTP 3′,8-cyclase (Rattus norvegicus) | 85.70% |
| | | F1LV47 | Olfactory receptor (Rattus norvegicus) | 85.70% |
| | | A0A0G2K7P1 | AAA_12 domain-containing protein (Rattus norvegicus) | 100.00% |
| | | A0A0G2K7L6 | FYVE, RhoGEF and PH domain-containing 6 (Rattus | 100.00% |
| | | | norvegicus) | |
| | | F1LRZ0 | Tyrosine-protein kinase receptor (Rattus norvegicus) | 100.00% |
| | | Q2TGJ6 | Palmitoyltransferase (Rattus norvegicus) | 100.00% |
| | | E9PU37 | Palmitoyltransferase (Rattus norvegicus) | 100.00% |
| | | Q9EPR9 | Phosphodiesterase (Rattus norvegicus) | 100.00% |
| | | A0A0G2K2K1 | Phosphodiesterase (Rattus norvegicus) | 100.00% |
| | | A0A0G2JZX9 | Phosphodiesterase (Rattus norvegicus) | 100.00% |
| | | Q9EPS0 | Phosphodiesterase 1A (Rattus norvegicus) | 100.00% |
| | | D3ZAU0 | Mucin 5B, oligomeric mucus/gel-forming (Rattus norvegicus) | 85.70% |
| | | A0A0G2JUZ9 | Olfactory receptor (Rattus norvegicus) | 87.50% |
| | | D3ZB08 | Olfactory receptor (Rattus norvegicus) | 85.70% |
| A + | SLLTTGPV | D3ZAU0 | Mucin 5B, oligomeric mucus/gel-forming (Rattus norvegicus) | 100.00% |
| C1-6 | (SEQ ID | P97639 | Melanin-concentrating hormone receptor 1 (Rattus | 85.70% |
| | NO: 34) | | norvegicus) | |
| | | A0A0G2JWU6 | Uncharacterized protein (Rattus norvegicus) | 85.70% |
| | | P53670 | LIM domain kinase 2 (Rattus norvegicus) | 85.70% |
| | | D3ZYM4 | Receptor protein-tyrosine kinase (Rattus norvegicus) | 83.30% |
| | | Q5D047 | LIM motif-containing protein kinase 2, isoform | 85.70% |
| | | | CRA_e (Rattus norvegicus) | |
| A + | SGSMIAMP | Q5I0K3 | Citramalyl-CoA lyase, mitochondrial (Rattus norvegicus) | 85.70% |
| C1-11- | (SEQ ID | A0A0G2K3H2 | Dedicator of cytokinesis 7 (Rattus norvegicus) | 63.60% |
| | NO: 35) | F1LRS2 | Dedicator of cytokinesis 7 (Rattus norvegicus) | 63.60% |
| | | G4XKZ2 | Ubiquitinyl hydrolase 1 (Rattus norvegicus) | 71.40% |
| | | A0A0G2JZU8 | Ubiquitin-specific peptidase 9, Y-linked (Rattus norvegicus) | 71.40% |
| | | W8CC82 | Ubiquitinyl hydrolase 1 (Rattus norvegicus) | 71.40% |
| | | A0A1B0GWR2 | Ubiquitin-specific peptidase 9, Y-linked (Rattus norvegicus) | 71.40% |
| | | P30823 | High affinity cationic amino acid transporter 1 (Rattus | 71.40% |
| | | | norvegicus) | |
| | | O08812 | Cationic amino acid transporter 3 (Rattus norvegicus) | 71.40% |
| | | Q6NX65 | Programmed cell death protein 10 (Rattus norvegicus) | 66.70% |
| | | A0A0G2K3I1 | High affinity cationic amino acid transporter 1 (Rattus | 71.40% |
| | | | norvegicus) | |
| | | Q3V5G8 | Cationinc amino acid transporter 1 (Rattus norvegicus) | 71.40% |
| | | Q8VIA9 | Ecotropic retrovirus receptor (Rattus norvegicus) | 71.40% |
| | | P70608 | Cationic amino acid transporter-1 (Rattus norvegicus) | 71.40% |
| | | G3V6I8 | Cationic amino acid transporter 3 (Rattus norvegicus) | 71.40% |
| | | O55043 | Rho guanine nucleotide exchange factor 7 (Rattus norvegicus) | 100.00% |
| | | A0A0G2QC21 | Rho guanine nucleotide exchange factor 7 (Rattus norvegicus) | 100.00% |
| | | A3KMH4 | Arhgef7 protein (Rattus norvegicus) | 100.00% |
| | | Q52NK0 | PAK-interacting exchange factor (Rattus norvegicus) | 100.00% |
| | | Q923I5 | PAK-interacting exchange factor beta2-PIX (Rattus | 100.00% |
| | | | norvegicus) | |
| | | Q5J3G7 | Vomeronasal type-1 receptor (Rattus norvegicus) | 100.00% |
| | | Q5J3F8 | Vomeronasal type-1 receptor (Rattus norvegicus) | 100.00% |
| A + | SSAGMHVL | A0A0G2K8R8 | Uncharacterized protein (Rattus norvegicus) | 75.00% |
| C1-13- | (SEQ ID | O55171 | Acyl-coenzyme A thioesterase 2, mitochondrial (Rattus | 85.70% |
| | NO: 36) | | norvegicus) | |
| | | O88267 | Acyl-coenzyme A thioesterase 1 (Rattus norvegicus) | 85.70% |
| | | Q6IMX8 | Acyl-CoA thioesterase 2 (Rattus norvegicus) | 85.70% |
| | | A0A0G2K7Z3 | Acyl-coenzyme A thioesterase 1 (Rattus norvegicus) | 85.70% |
| | | D4AA47 | Phospholipid-transporting ATPase IC (Rattus norvegicus) | 85.70% |
| | | F7FF30 | Lon protease homolog 2, peroxisomal (Rattus norvegicus) | 100.00% |
| | | Q6TXI3 | LRRGT00016 (Rattus norvegicus) | 100.00% |
| | | A0A0G2K3S9 | Solute carrier family 24 member 4 (Rattus norvegicus) | 85.70% |
| A + | SHNSQSIP | D3ZEH1 | FAT atypical cadherin 4 (Rattus norvegicus) | 71.40% |
| C1-16- | (SEQ ID | D4A5W5 | DNA helicase (Rattus norvegicus) | 71.40% |
| | NO: 37) | P43244 | Matrin-3 (Rattus norvegicus) | 85.70% |
| | | A0A0G2JSR7 | Matrin-3 (Rattus norvegicus) | 85.70% |
| | | B5DEM0 | Matr3 protein (Rattus norvegicus) | 85.70% |
| | | Q6P6H1 | Matr3 protein (Rattus norvegicus) | 85.70% |
| A + | SLGMTYGH | A0A0U1RRV0 | Praja ring finger ubiquitin ligase 1 (Rattus norvegicus) | 83.30% |
| C1-22- | (SEQ ID | A0A0U1RRZ8 | Praja ring finger ubiquitin ligase 1 (Rattus norvegicus) | 83.30% |
| | NO: 38) | Q5U2T6 | Zinc finger protein PLAG1 (Rattus norvegicus) | 83.30% |
| | | F1M1H4 | Vomeronasal 2 receptor, 79 (Rattus norvegicus) | 83.30% |
| | | F1LVA5 | RAS and EF hand domain-containing (Rattus norvegicus) | 83.30% |
| | | A0A0G2K4R8 | Reverse transcriptase domain-containing protein (Rattus | 83.30% |
| | | | norvegicus) | |
| | | B2GUT8 | Phosphatidylinositol N-acetylglucosaminyltransferase subunit | 83.30% |
| | | | P (Rattus norvegicus) | |
| A + | SMPDASLS | O54852 | Potassium voltage-gated channel subfamily H member | 75.00% |
| C2-5 | (SEQ ID | | 7 (Rattus norvegicus) | |
| | NO: 39) | D3ZX21 | AP-4 complex subunit epsilon (Rattus norvegicus) | 85.70% |
| | | A0A0H2UHF2 | Potassium voltage-gated channel subfamily H member | 75.00% |
| | | | 7 (Rattus norvegicus) | |
| | | A0A0G2K3B8 | Potassium voltage-gated channel subfamily H member | 75.00% |
| | | | 7 (Rattus norvegicus) | |
| | | E9PSU6 | Pecanex-like protein 1 (Rattus norvegicus) | 100.00% |
| | | B1WC89 | Ankrd58 protein (Rattus norvegicus) | 100.00% |
| | | P53676 | AP-3 complex subunit mu-1 (Rattus norvegicus) | 85.70% |
| | | D3ZGS1 | Vomeronasal 2 receptor, 54 (Rattus norvegicus) | 83.30% |
| | | Q6IRG9 | AP-3 complex subunit mu-1 (Rattus norvegicus) | 85.70% |
| | | F1MAK3 | Rho GTPase-activating protein 32 (Rattus norvegicus) | 100.00% |
| | | B5DEI5 | Rics protein (Rattus norvegicus) | 100.00% |
| | | P70478 | Adenomatous polyposis coli protein (Rattus norvegicus) | 75.00% |
| | | A0A0G2K8G0 | Adenomatous polyposis coli protein (Rattus norvegicus) | 75.00% |
| | | G3V8Q9 | Adenomatosis polyposis coli, isoform CRA_a (Rattus | 75.00% |
| | | | norvegicus) | |
| A + | SQQTDSYP | E9PT35 | Zinc finger protein 128 (Rattus norvegicus) | 100.00% |
| C2-16- | (SEQ ID | Q0KL00 | Piezo-type mechanosensitive ion channel component | 75.00% |
| | NO: 40) | | 1 (Rattus norvegicus) | |
| | | A0A0G2JWS3 | Piezo-type mechanosensitive ion channel component (Rattus | 75.00% |
| | | | norvegicus) | |
| | | D3ZTU9 | Arylacetamide deacetylase-like 4 (Rattus norvegicus) | 100.00% |
| A + | SASVHPLA | P54258 | Atrophin-1 (Rattus norvegicus) | 100.00% |
| C2-17- | (SEQ ID | G3V7W3 | Atrophin-1 (Rattus norvegicus) | 100.00% |
| | NO: 41) | F1M388 | Uncharacterized protein (Rattus norvegicus) | 80.00% |
| | | Q6AY78 | Solute carrier family 22 member 18 (Rattus norvegicus) | 100.00% |
| | | F1M4Q7 | SET-binding protein 1 (Rattus norvegicus) | 85.70% |
| | | A0A0G2JSS3 | Solute carrier family 22 member 18 (Rattus norvegicus) | 100.00% |
| | | Q499U2 | Engulfment and cell motility protein 3 (Rattus norvegicus) | 85.70% |
| | | Q5BJP4 | RNA binding motif protein 39 (Rattus norvegicus) | 85.70% |
| | | Q5M7W2 | Similar to palladin CGI-151 protein (Rattus norvegicus) | 85.70% |
| | | A0A096MJT6 | Similar to RIKEN cDNA D630029K19 (Rattus norvegicus) | 77.80% |
| | | F1LVK0 | Similar to RIKEN cDNA D630029K19 (Rattus norvegicus) | 77.80% |
| | | A0A0G2K1D4 | Similar to RIKEN cDNA D630029K19 (Rattus norvegicus) | 77.80% |
| | | Q5QD23 | Trace amine-associated receptor 5 (Rattus norvegicus) | 100.00% |
| | | D4ABX7 | Similar to dystonin isoform 1 (Rattus norvegicus) | 83.30% |
| | | D3ZW47 | Tripartite motif-containing 16 (Rattus norvegicus) | 100.00% |
| | | D8KZT4 | RCG42029 (Rattus norvegicus) | 100.00% |
| A + | SWTAYMSS | Q6AY01 | E3 ubiquitin-protein ligase RNF133 (Rattus norvegicus) | 100.00% |
| C2-21- | (SEQ ID | D3ZH90 | NME/NM23 family member 5 (Rattus norvegicus) | 100.00% |
| | NO: 42) | O70240 | Axin-2 (Rattus norvegicus) | 100.00% |
| | | P31430 | Dipeptidase 1 (Rattus norvegicus) | 80.00% |
| | | F1M495 | Mannose receptor, C type 2 (Predicted) (Rattus norvegicus) | 80.00% |
| | | A0A0G2K457 | Phospholipase A2 receptor 1 (Rattus norvegicus) | 80.00% |
| | | A0A0G2K4L4 | Axin-2 (Rattus norvegicus) | 100.00% |
| | | Q9JJH5 | 6-phosphofructo-2-kinase/fructose-2,6-bisphosphatase | 66.70% |
| | | | 2 (Rattus norvegicus) | |
| | | P0C0L6 | Neuropeptide S receptor (Rattus norvegicus) | 80.00% |
| | | D3Z822 | Non-specific serine/threonine protein kinase (Rattus | 80.00% |
| | | | norvegicus) | |
| | | F1M400 | Ankyrin repeat domain 28 (Rattus norvegicus) | 80.00% |
| | | F1LZH9 | Ankyrin repeat domain 44 (Rattus norvegicus) | 80.00% |
| | | A0A0G2JWZ9 | Ankyrin repeat domain 44 (Rattus norvegicus) | 80.00% |
| | | R9PXY6 | 6-phosphofructo-2-kinasc (Rattus norvegicus) | 66.70% |
| | | A0A0H2UHN0 | Neuropeptide S receptor (Rattus norvegicus) | 80.00% |
| | | D0EA89 | 6-phosphofructo-2-kinase/fructose-2, 6-bisphosphatase-2 | 66.70% |
| | | | variant 7 (Rattus norvegicus) | |
| | | G3V7K5 | NPC intracellular cholesterol transporter 1 (Rattus | 80.00% |
| | | | norvegicus) | |
| | | D3Z8C9 | Phosphodiesterase (Rattus norvegicus) | 80.00% |
| | | F1M8K7 | Dol-P-Man:Man(5)GlcNAc(2)-PP-Dol alpha-1,3- | 80.00% |
| | | | mannosyltransferase (Rattus norvegicus) | |
| A + | SPQLTRNV | A0A0G2K916 | Ankyrin repeat and KH domain-containing 1 (Rattus | 100.00% |
| C2-22- | (SEQ ID | | norvegicus) | |
| | NO: 43) | D4A885 | ATP-binding cassette subfamily A member 13 (Rattus | 83.30% |
| | | | norvegicus) | |
| | | A0A0G2JUI2 | RUN and FYVE domain-containing 4 (Rattus norvegicus) | 100.00% |
| | | F1M513 | RUN and FYVE domain-containing 4 (Rattus norvegicus) | 100.00% |
| | | Q6MG63 | Ng23 protein (Rattus norvegicus) | 100.00% |
| A + | SHAHSLSV | Q7TQ89 | Ac1055 (Rattus norvegicus) | 100.00% |
| C2-28- | (SEQ ID | D3ZG85 | Cyclin-dependent kinase-like 5 (Rattus norvegicus) | 85.70% |
| | NO: 44) | E2E1S0 | Cyclin-dependent kinase-like 5 (Rattus norvegicus) | 85.70% |
| | | D3ZQK8 | Lymphocyte antigen 75 (Rattus norvegicus) | 100.00% |
| | | E9PU85 | Asteroid homolog 1 (Rattus norvegicus) | 100.00% |
| | | A0A0G2K873 | Asteroid homolog 1 (Rattus norvegicus) | 100.00% |
| | | A0A0G2K212 | Non-specific serine/threonine protein kinase (Rattus | 83.30% |
| | | | norvegicus) | |
| | | D3ZAR2 | Non-specific serine/threonine protein kinase (Rattus | 83.30% |
| | | | norvegicus) | |
| | | Q5J3D7 | Vomeronasal type-1 receptor (Rattus norvegicus) | 83.30% |
| A + | SNPYSPSL | D4A5I4 | Neuron navigator 1 (Rattus norvegicus) | 75.00% |
| C2-32- | (SEQ ID | F1LYK3 | Neuron navigator 1 (Rattus norvegicus) | 75.00% |
| | NO: 45) | F1M031 | Neuron navigator 1 (Rattus norvegicus) | 75.00% |
| | | D3ZXP1 | Tetratricopeptide repeat domain 28 (Rattus norvegicus) | 83.30% |
| | | B1WBR8 | F-box and leucine-rich repeat protein 4 (Rattus norvegicus) | 100.00% |
| | | P07897 | Aggrecan core protein (Rattus norvegicus) | 83.30% |
| | | D4A7Y1 | Aggrecan core protein (Rattus norvegicus) | 83.30% |
| | | F1LQI4 | Aggrecan core protein (Rattus norvegicus) | 83.30% |
| | | F1M5G8 | Transmembrane protein 131 (Rattus norvegicus) | 83.30% |
| | | O54968 | Nuclear factor erythroid 2-related factor 2 (Rattus norvegicus) | 100.00% |
| | | D3ZTX4 | Maltase-glucoamylase (Rattus norvegicus) | 83.30% |
| | | Q4QYD9 | Heat sensitive channel TRPV3 (Rattus norvegicus) | 85.70% |
| | | E9PU00 | Transient receptor potential cation channel, subfamily V, | 85.70% |
| | | | member 3 (Rattus norvegicus) | |
| | | A0A0G2JYM0 | LIM domain-binding 3 (Rattus norvegicus) | 100.00% |
| | | A0A096MJ01 | LIM domain-binding 3 (Rattus norvegicus) | 100.00% |
| | | A0A096MKD4 | LIM domain-binding 3 (Rattus norvegicus) | 100.00% |
| | | A0A0G2JZW6 | Uncharacterized protein (Rattus norvegicus) | 85.70% |
| A + | SYLPDEFT | Q566R2 | TM2 domain-containing protein 2 (Rattus norvegicus) | 100.00% |
| C3-6- | (SEQ ID | A0JN29 | Endoplasmic reticulum junction formation protein | 100.00% |
| | NO: 46) | | lunapark (Rattus norvegicus) | |
| | | P49655 | ATP-sensitive inward rectifier potassium channel 10 (Rattus | 50.00% |
| | | | norvegicus) | |
| | | A0A096MJX4 | Fibrous sheath-interacting protein 2 (Rattus norvegicus) | 83.30% |
| | | A0A0G2K296 | Fibrous sheath-interacting protcin 2 (Rattus norvegicus) | 83.30% |
| | | A0A0U1RS25 | UPF1, RNA helicase and ATPase (Rattus norvegicus) | 85.70% |
| | | F1LY19 | RCG38820 (Rattus norvegicus) | 85.70% |
| | | A0A0H2UHF5 | ATP-sensitive inward rectifier potassium channel 10 (Rattus | 50.00% |
| | | | norvegicus) | |
| | | P49621 | Diacylglycerol kinase beta (Rattus norvegicus) | 83.30% |
| | | A0A0G2K4Y6 | HAUS6_N domain-containing protein (Rattus norvegicus) | 83.30% |
| | | F1LP01 | Diacylglycerol kinase (Rattus norvegicus) | 83.30% |
| | | F7EXD0 | DET1 partner of COP1 E3 ubiquitin ligase (Rattus | 100.00% |
| | | | norvegicus) | |
| | | Q498E1 | De-etiolated homolog 1 (Arabidopsis) (Rattus norvegicus) | 100.00% |
| | | G3V6G8 | Protein kinase, membrane associated tyrosine/threonine 1 | 85.70% |
| | | | (Predicted) (Rattus norvegicus) | |
| | | B5DFC5 | Pkmyt1 protein (Rattus norvegicus) | 85.70% |
| | | D3ZJL1 | Malignant fibrous histiocytoma amplified sequence 1 | 100.00% |
| | | | (Predicted), isoform CRA_a (Rattus norvegicus) | |
| | | B2RYH5 | Interleukin-1 receptor-associated kinase 1 (Rattus norvegicus) | 66.70% |
| | | A0A0G2K3K5 | Beta-1,4-galactosyltransferase 5 (Rattus norvegicus) | 57.10% |
| | | F1LZL3 | Beta-1,4-galactosyltransferase 5 (Rattus norvegicus) | 57.10% |
| A + | STIQFTPQ | D3ZWZ1 | Integrin subunit alpha X (Rattus norvegicus) | 100.00% |
| C4-1* | (SEQ ID | D4A542 | G protein-coupled receptor-associated sorting protein | 83.30% |
| | NO: 47) | | 2 (Rattus norvegicus) | |
| | | A0A0G2JWC7 | Fermitin family member 2 (Rattus norvegicus) | 100.00% |
| | | Q5XI19 | Fermitin family homolog 2 (Drosophila) (Rattus norvegicus) | 100.00% |
| | | A0A0G2JYT2 | Stabilizer of axonemal microtubules 2 (Rattus norvegicus) | 100.00% |
| | | D3ZDP0 | Stabilizer of axonemal microtubules 2 (Rattus norvegicus) | 100.00% |
| | | D3ZL91 | Uncharacterized protein (Rattus norvegicus) | 85.70% |
| A + | STLSFTRM | D4AE71 | Similar to BC067074 protein (Rattus norvegicus) | 100.00% |
| C4-2* | (SEQ ID | Q66HD2 | Kelch-like protein 36 (Rattus norvegicus) | 100.00% |
| | NO: 48) | Q06226 | Serine/threonine-protein kinase Sgk1 (Rattus norvegicus) | 62.50% |
| | | D4A599 | Dynein, axonemal, heavy chain 17 (Rattus norvegicus) | 62.50% |
| | | Q68G05 | Serine/threonine-protein kinase Sgk1 (Rattus norvegicus) | 62.50% |
| | | Q811T1 | Serine/threonine protein kinase SGK (Rattus norvegicus) | 62.50% |
| | | Q9R283 | Short transient receptor potential channel 2 (Rattus | 75.00% |
| | | | norvegicus) | |
| | | A0A140TAF6 | Short transient receptor potential channel 2 (Rattus | 75.00% |
| | | | norvegicus) | |
| | | A0A096MK89 | Adhesion G protein-coupled receptor V1 (Rattus norvegicus) | 83.30% |
| | | A0A0G2JUP2 | Adhesion G protein-coupled receptor V1 (Rattus norvegicus) | 83.30% |
| | | A9NI05 | Neurepin (Rattus norvegicus) | 83.30% |
| A + | STWSYPRI | D3Z5V4 | Family with sequence similarity 124, member A (Mus | 80.00% |
| C4-6 | (SEQ ID | | musculus) | |
| | NO: 49) | | | |
| A + | SPLSFQAT | O70156 | Oxidized low-density lipoprotein receptor 1 (Rattus | 100.00% |
| C4-9 | (SEQ ID | | norvegicus) | |
| | NO: 50) | A0A0G2K1K0 | Oxidized low density lipoprotein (Lectin-like) receptor 1, | 100.00% |
| | | | isoform CRA_a (Rattus norvegicus) | |
| | | Q9R1R4 | Tudor domain-containing protein 7 (Rattus norvegicus) | 100.00% |
| | | P49744 | Thrombospondin-4 (Rattus norvegicus) | 100.00% |
| | | A0A0G2K7L8 | Thrombospondin-4 (Rattus norvegicus) | 100.00% |
| | | F1LMS5 | Thrombospondin-4 (Rattus norvegicus) | 100.00% |
| | | D3ZG75 | ALS2 C-terminal-like (Rattus norvegicus) | 83.30% |
| | | D4A9H7 | Similar to RIKEN cDNA 2610039E05 (Predicted), isoform | 83.30% |
| | | | CRA_a (Rattus norvegicus) | |
| A + | SNFPSLSV | B0BNK8 | Terminal nucleotidyltransferase 5B (Rattus norvegicus) | 85.70% |
| C4-16 | (SEQ ID | D3ZIP2 | CD180 antigen (Predicted), isoform CRA_a (Rattus | 100.00% |
| | NO: 51) | | norvegicus) | |
| | | D3ZYM4 | Receptor protein-tyrosine kinase (Rattus norvegicus) | 100.00% |
| | | Q5BK80 | Tetraspanin (Rattus norvegicus) | 85.70% |
| | | Q8VIJ5 | Protein O-GlcNAcase (Rattus norvegicus) | 100.00% |
| | | P69527 | Aminopeptidase O (Rattus norvegicus) | 100.00% |
| | | Q5FVM3 | Reticulophagy regulator 1 (Rattus norvegicus) | 83.30% |
| | | B0BND0 | Glycerophosphocholinecholinephosphodiesterase | 83.30% |
| | | | ENPP6 (Rattus norvegicus) | |
| | | A0A0H2UI10 | Protein O-GlcNAcase (Rattus norvegicus) | 100.00% |
| | | A0A0G2K4L3 | Zinc finger protein 622 (Rattus norvegicus) | 83.30% |
| | | D3ZRI1 | Choline-specific glycerophosphodiester | 83.30% |
| | | | phosphodiesterase (Rattus norvegicus) | |
| | | A0A0G2JUQ3 | Protein FAM134B (Rattus norvegicus) | 83.30% |
| | | Q63155 | Netrin receptor DCC (Rattus norvegicus) | 83.30% |
| | | Q9QX72 | Selenocysteine insertion sequence-binding protein 2 (Rattus | 83.30% |
| | | | norvegicus) | |
| | | F1LRN5 | Netrin receptor DCC (Rattus norvegicus) | 83.30% |
| | | F1LPQ8 | SECIS binding protein 2, isoform CRA_a (Rattus norvegicus) | 83.30% |
| | | D3ZL57 | USP6 N-terminal like (Predicted), isoform CRA_a (Rattus | 83.30% |
| | | | norvegicus) | |
| A + | SNKMDNTL | D4A3W8 | Dishevelled-binding antagonist of beta-catenin 1 (Rattus | 83.30% |
| C4-21 | (SEQ ID | | norvegicus) | |
| | NO: 52) | Q5U2P2 | Immunoglobulin superfamily member 11 (Rattus norvegicus) | 83.30% |
| A + | SPIRGYTD | D3ZJK0 | NAD(P)(+)--arginine ADP-ribosyltransferase (Rattus | 100.00% |
| C4-25 | (SEQ ID | | norvegicus) | |
| | NO: 53) | Q5U2P0 | DIS3-like exonuclease 1 (Rattus norvegicus) | 75.00% |
| | | D4ADZ6 | Helicase with zinc finger (Rattus norvegicus) | 83.30% |
| | | Q6AY04 | m-AAA protease-interacting protein 1, mitochondrial (Rattus | 83.30% |
| | | | norvegicus) | |
| | | F7EPZ4 | DIS3 homolog, exosome endoribonuclease and 3′-5′ | 75.00% |
| | | | exoribonuclease (Rattus norvegicus) | |
| | | B2RYL7 | Dis3 protein (Rattus norvegicus) | 75.00% |
| A + | SVDDYFTR | Q63633 | Solute carrier family 12 member 5 (Rattus norvegicus) | 83.30% |
| C5-1* | (SEQ ID | F1LNP4 | Solute carrier family 12 member 5 (Rattus norvegicus) | 83.30% |
| | NO: 54) | D3ZGI9 | Solute carrier family 12 member 5 (Rattus norvegicus) | 83.30% |
| | | Q5JCS6 | Signal-induced proliferation-associated 1-like protein | 83.30% |
| | | | 2 (Rattus norvegicus) | |
| | | F1M8G8 | Signal-induced proliferation-associated 1-like protein | 83.30% |
| | | | 2 (Rattus norvegicus) | |
| | | B1WBV6 | Aplp1 protein (Rattus norvegicus) | 100.00% |
| | | F1LRS5 | Amyloid beta precursor-like protein 1 (Rattus norvegicus) | 100.00% |
| | | D3ZEV3 | MGAT4 family, member C (Rattus norvegicus) | 83.30% |
| | | I6LBX6 | Protocadherin gamma a9 (Rattus norvegicus) | 83.30% |
| | | E9PT60 | Protocadherin gamma subfamily A, 9 (Rattus norvegicus) | 83.30% |
| | | D4AAG9 | DNA helicase (Rattus norvegicus) | 57.10% |
| | | A0A0G2K5T4 | DNA helicase (Rattus norvegicus) | 57.10% |
| | | Q9QUR2 | Dynactin subunit 4 (Rattus norvegicus) | 66.70% |
| | | Q498N3 | Dctn4 protein (Rattus norvegicus) | 66.70% |
| | | F1M5X2 | AP complex subunit sigma (Rattus norvegicus) | 66.70% |
| | | B0BN62 | AP complex subunit sigma (Rattus norvegicus) | 66.70% |
| A + | SIDYMYAR | O35764 | Neuronal pentraxin receptor (Rattus norvegicus) | 58.30% |
| C5-3 | (SEQ ID | F1LSY2 | Neuronal pentraxin receptor (Rattus norvegicus) | 58.30% |
| | NO: 55) | O54735 | cGMP-specific 3′,5′-cyclic phosphodiesterase (Rattus | 83.30% |
| | | | norvegicus) | |
| | | G3V7V8 | Phosphodiesterase (Rattus norvegicus) | 83.30% |
| | | D3ZPJ1 | Kelch-like 11 (Drosophila) (Predicted) (Rattus norvegicus) | 80.00% |
| | | D4A264 | Zinc-binding alcohol dehydrogenase, domain-containing | 80.00% |
| | | | 2 (Rattus norvegicus) | |
| | | Q5M9F8 | N-terminal kinase-like protein (Rattus norvegicus) | 100.00% |
| | | P47971 | Neuronal pentraxin-1 (Rattus norvegicus) | 80.00% |
| | | A0A1W2Q655 | Zinc finger and BTB domain containing 20 (Predicted), | 80.00% |
| | | | isoform CRA_c (Rattus norvegicus) | |
| | | D4A1U1 | Zinc finger and BTB domain containing 20 (Predicted), | 80.00% |
| | | | isoform CRA_b (Rattus norvegicus) | |
| | | D3ZTY1 | BTB (POZ) domain containing 4 (Predicted) (Rattus | 80.00% |
| | | | norvegicus) | |
| | | A0A0H2UHC1 | Neuronal pentraxin-1 (Rattus norvegicus) | 80.00% |
| | | Q5U2U9 | Poly(A)-specific ribonuclease (Rattus norvegicus) | 83.30% |
| | | D3ZYT8 | 1-phosphatidylinositol-3-phosphate 5-kinase (Rattus | 66.70% |
| | | | norvegicus) | |

TABLE 11
Summary of Protein BLAST Identities for Primed Astrocytes

| Code | Peptide | Entry | Description | Homology |
|---|---|---|---|---|
| AD | STTVNTIA | D3ZXT1 | Leucine rich repeat containing 4C (Predicted), isoform | 100.00% |
| C4-11- | (SEQ ID | | CRA_a (Rattus norvegicus) | |
| | NO: 56) | A0A0G2JXL5 | Uncharacterized protein (Rattus norvegicus) | 87.50% |
| | | M0R3P0 | Uncharacterized protein (Rattus norvegicus) | 87.50% |
| | | A0A1Y1FJE1 | Mucin apoprotein (Rattus norvegicus) | 87.50% |
| | | Q9JLA3 | UDP-glucose:glycoprotein glucosyltransferase 1 (Rattus | 83.30% |
| | | | norvegicus) | |
| | | Q9QWI0 | Peroxisome proliferator activated receptor delta (Rattus | 71.40% |
| | | | norvegicus) | |
| | | D3ZM14 | Partner and localizer of BRCA2 (Rattus norvegicus) | 83.30% |
| AD | STYHTNSS | Q62799 | Receptor tyrosine-protein kinase erbB-3 (Rattus norvegicus) | 83.30% |
| C5-9- | (SEQ ID | G3V6N1 | Receptor protein-tyrosine kinase (Rattus norvegicus) | 83.30% |
| | NO: 57) | | | |
| AD | SGSSFTMI | Q6AXT2 | Similar to RIKEN cDNA 2810408A11 (Rattus norvegicus) | 100.00% |
| C6-5- | (SEQ ID | F1LP90 | Misshapen-like kinase 1 (Rattus norvegicus) | 100.00% |
| | NO: 58) | Q6AY30 | Saccharopine dehydrogenase-like oxidoreductase (Rattus | 100.00% |
| | | | norvegicus) | |
| | | F1LN69 | Misshapen-like kinase 1 (Rattus norvegicus) | 100.00% |
| | | A0A0G2JV77 | Misshapen-like kinase 1 (Rattus norvegicus) | 100.00% |
| | | A0A0G2K382 | Misshapen-like kinase 1 (Rattus norvegicus) | 100.00% |
| | | Q9R0S5 | Y + L amino acid transporter 1 (Rattus norvegicus) | 83.30% |

1. A biopanning method for identifying peptides that specifically bind to one or more glial cell sub-types, the method comprising:
a) providing a population of surveying glial cells, a population of reactive glial cells, and a population of primed glial cells;
b) contacting each population with a phage library;
c) isolating phage-bound glial cells; and
sequencing the phage bound-glial cells.

2. The method of claim 1, further comprising amplifying the isolated phage-bound glial cells prior to sequencing.

3. The method of claim 1, wherein the population of surveying glial cells comprises cells having one or more of the following characteristics:
bifurcated processes extending from the cell body; and/or
no detectable iNOS expression.

4. The method of claim 1, wherein the population of reactive glial cells comprises cells having one or more of the following characteristics:
a flattened and/or ameboid shape;
increased release of one or more pro-inflammatory proteins compared to release from primed glial cells or surveying glial cells;
increased release of nitric oxide (NO) compared to release from primed glial cells or surveying glial cells; and/or
increased expression of inducible nitric oxide synthase (iNOS) compared to expression of iNOS in primed glial cells or surveying glial cells.

5. The method of claim 4, wherein the one or more pro-inflammatory proteins are selected from TNF-α, IL-1β, and IL-10.

6. The method of claim 1, wherein the population of primed glial cells comprises cells having one or more of the following characteristics:
a less flattened nature than reactive glial cells and a smaller number of processes extending from the cell body than in surveying glial cells;
a higher expression of iNOS than in surveying glial cells and a lower expression of iNOS than in reactive glial cells.

7. The method of claim 1, wherein the population of reactive glial cells is generated by culturing glial cells in a composition comprising lipopolysaccharide (LPS), and/or the population of primed microglial cells is generated by culturing glial cells in a composition comprising lipopolysaccharide (LPS), followed by culturing the glial cells in a composition comprising dexamethasone.

8. The method of claim 7, wherein the composition comprises LPS at a concentration of about 10 to about 200 ng/mL.

9. The method of claim 8, wherein the composition comprises LPS at a concentration of about 50 to about 150 mg/mL.

10. The method of claim 7, wherein the glial cells are cultured in the composition comprising LPS for 4-8 hours.

11. (canceled)

12. The method of claim 7, wherein the composition comprising LPS comprises LPS at a concentration of about 10 to about 200 ng/mL, and wherein the composition comprising dexamethasone comprises dexamethasone at a concentration of about 1 μM to about 20 μM.

13. The method of claim 12, wherein the composition comprising LPS comprises LPS at a concentration of about 50 to about 150 ng/mL, and wherein the composition comprising dexamethasone comprises dexamethasone at a concentration of about 5 μM to about 15 μM.

14. The method of claim 7, wherein the population of primed microglial cells is generated by culturing the glial cells in the composition comprising LPS for 4-8 hours, followed by culturing the glial cells in the composition comprising dexamethasone for 12-36 hours.

15. The method of claim 1, wherein the phage library comprises a filamentous phage library.

16. The method of claim 1, wherein the phage library comprises a plurality of phages, each phage displaying one or more peptides comprising 7 randomized amino acids.

17. The method of claim 1, wherein the phage library comprises an M13 phage library.

18. The method of claim 16, wherein the one or more peptides have at least 62.5% sequence identity to a sequence selected from SEQ ID NO: 1-SEQ ID NO: 58.

19. The method of claim 18, wherein the one or more peptides have at least 75% sequence identity to a sequence selected from SEQ ID NO: 1-SEQ ID NO: 58.

20. The method of claim 19, wherein the one or more peptides each have at least 87.5% sequence identity to a sequence selected from SEQ ID NO: 1-SEQ ID NO: 58.

21. The method of claim 18, wherein the one or more peptides are exactly 7 amino acids in length.

22. (canceled)
